(19) United States
(12) Patent Application Publication
(10) Pub. No.: US 2026/0216073 A1
(43) Pub. Date: Jul. 30, 2026

(54) MANUFACTURING PROCESS FOR HYBRID LIPID PARTICLES

(71) Applicant: SiSaf Ltd, Guildford Surrey (GB)

(72) Inventors: Roghieh Suzanne SAFFIE-SIEBERT, Guildford Surrey (GB); Nasrollah TORABI-POUR, Guildford Surrey (GB); Ashkan DEHSORKHI, Guildford Surrey (GB)

(73) Assignee: SiSaf Ltd, Guildford Surrey (GB)

(21) Appl. No.: 19/148,624

(22) PCT Filed: Jan. 19, 2024
(86) PCT No.: PCT/GB2024/050158

§ 371 Date: Jul. 16, 2025

(30) Foreign Application Priority Data
Jan. 20, 2023 (GB) 2300914.5

(51) Int. Cl. A61K 9/1272; A61K 9/1277; A61K 9/19; A61K 9/51; A61K 31/7105; B82Y 5/00

(52) U.S. Cl. CPC A61K 9/1272; A61K 9/1277; A61K 9/19; A61K 9/5115; A61K 9/5123; A61K 31/7105; B82Y 5/00

(57) ABSTRACT

Method of manufacturing an aqueous suspension of hybrid lipid particles comprising the steps of mixing one or more lipids in a solvent or solvent mixture and a suspension of particles of inorganic material in a solvent or solvent mixture, into an aqueous medium; then passing the mixture resulting from Step A through the pores of an extrusion membrane, wherein the hybrid lipid particles have a mean diameter at least two times larger than the mean diameter of the particles of inorganic material. Related product, methods of treatment and medical application.

### FIELD OF THE INVENTION

The invention relates to hybrid lipid particles and to methods of manufacturing hybrid lipid particles. In particular, it relates to methods of manufacturing hybrid lipid particles comprising the use of hydrolysable silicon particles to stabilise the hybrid lipid particles in an intermediate state to which an active compound such as an API may be added to form a hybrid lipid particle carrier for that active compound. In particular, it relates to scalable methods of producing hybrid lipid particles especially suitable for delivery of nucleic acid compounds. The invention further relates to products produced by or otherwise related to the methods of the invention.

In certain aspects, the invention further relates to a convergent manufacturing approach employed for the production of hybrid lipid particles for use as a carrier for an active compound in which empty carrier is initially produced and purified before an active compound such as a nucleic acid is loaded in a separate step, normally as part of the fill and finish operation.

### BACKGROUND OF THE INVENTION

Lipid nanoparticles (LNPs) have revolutionized the field of nucleic acid therapeutics by overcoming significant challenges in the cellular delivery of DNA and RNA. They particularly rose to prominence as the mRNA delivery technology in the Moderna and Pfizer/BioNTech COVID-19 vaccines (Wang et al., 2021), and currently represent the predominant delivery mechanism in over 200 ongoing clinical trials of other RNA-based drugs (Curreri et al., 2023). Despite these impressive successes, current LNP and in general liposomal vesicles formulations face some recognized shortcomings that must be overcome to realize improved clinical translation of RNA-based therapeutics (Moss et al., 2019; Verma et al., 2023).

One crucial limitation concerns the inherent chemical and metabolic lability of RNA, particularly its hydrolytic instability (Schoenmaker et al., 2021), which presents significant challenges for the larger mRNA constructs representing the majority of products in clinical development (Curreri et al., 2023). As a result, most marketed nucleic acid therapeutics contain extensive chemical modifications to enhance stability and efficacy (Bege & Borbás, 2022; Egli & Manoharan, 2023). When LNPs are used as the delivery vehicle, another issue that emerges is the need to introduce the RNA payload early in the production process. Effectively, LNPs must be formed around the RNA because there is no effective way of encapsulating it later (Nag et al., 2022; Cameau et al., 2022). This restricts batch sizes in commercial manufacturing due to RNA instability (Catignol & Lim, 2022), and it can also compromise product quality. Tozinameran, the mRNA-based Pfizer/BioNTech COVID-19 vaccine, is estimated to retain only 70% of initial mRNA integrity at the end of the manufacturing process (Daniel et al., 2022). Moreover, the currently marketed mRNA products (tozinameran, elasomeran, and derivatives) require cold chain distribution and storage to make them clinically and commercially viable. This is not ideal for sustainability or global accessibility in the long run (Andoh & Yu, 2023; Khairi et al., 2022).

SiSaf has recently developed silicon-stabilised lipid nanoparticles as an alternative nanocarrier for therapeutic RNA. That carrier addresses the aforementioned challenges. Incorporation of hydrolysable silicon modifies the properties of the resultant nanoparticles in unique and advantageous ways, particularly conferring superior long-term stability (Saffie-Siebert et al., 2023) and readily enabling tuning of the formulation for targeted nucleic acid delivery. The present invention is based on an appreciation that silicon-stablished hybrid lipid nanoparticles can be manufactured in “empty” form, shipped at ambient temperature (for example, in liquid suspension or as a lyophilized powder), and loaded with a desired therapeutic RNA at the point of use.

RNA is able to penetrate the lipid bilayer of vesicles at room temperature, unlike prior art lipid nanoparticles which require higher temperatures (~70-95° C. depending on the system). Silicon nanostructures facilitate the loading at RT, perhaps because the bilayer of lipids is discontinuous with silicon crossing the vesicle surface. Silicon particles are able to act as a Trojan horse to help RNA enter lipid bilayers.

Successful clinical translation of technology requires a reliable manufacturing process that can be deployed at scale. It is known that small changes in manufacturing a carrier for an active compound may influence the physical and chemical properties of both the carrier for an active compound, and the active compound itself which may influence the clinical profile of the active compound. Lipid particles—sometimes referred to as lipid nanoparticles, and especially liposomal lipid particles are of interest, especially for delivery of active pharmaceutical agents (APIs). They are also of interest for delivery of cosmetic, nutritional, nutraceutical, animal and plant health products and other purposes.

Micellar particles are approximately spherical in shape and are formed from lipids (especially lipids having hydrophilic “head” groups such as phospholipids—and also mixtures of lipids comprising lipids having hydrophobic head groups) packing together into supramolecular assemblies with the hydrophilic tails mutually attracted to each other and the hydrophilic heads facing outwards. Micelles have been used to encapsulate delicate APIs and thereby protect them from harsher external aqueous environments. For example, US2018/022151 discloses specific lipid particles suited to encapsulating APIs. Liposomal lipid particles are also approximately spherical in shape. They are formed from a bilayer of lipids and can be visualised as a lipid “bubble” surrounding an interior space which may be a hydrophilic environment. Alternatively, an aqueous environment may exist inside a liposomal lipid particle. Liposomal lipid particles are especially suitable for encapsulating active compounds.

It has been found that small lipid particles and especially small liposomal lipid particles are useful for protection of active compounds such as APIs and effective delivery of the active compound such as the API. Small particles in this context mean particles smaller than about 200, 150, 100 or 50 nm diameter. Liposomes may be especially useful in the delivery of active compounds which are hydrophilic because the internal space of a liposome may provide an aqueous environment for the active compound which is protected from the external aqueous environment. This may be especially advantageous, for example, if the external environment contains enzymes or other entities capable of damaging the active compound or API.

### DISCUSSION OF THE INVENTION

Small lipid particles, both those having liposomal characteristics and those having micellar characteristics, have a tendency to coalesce into larger particles as discussed in GB2210794.0. The inventors have found that particles of inorganic solids and, in particular, particles of hydrolysable silicon-containing material which themselves are smaller than the lipid particles, can be “dusted” onto the surface of the lipid and/or “into” the surface of the lipid particle (i.e., partially penetrating into the lipid particle but with a portion of the particles of inorganic material accessible on the surface), and thereby inhibit the tendency of the lipid particles to coalesce with each other, as well as coordinating with and thereby protecting any charged lipid constituents of the lipid particle and also providing the lipid particles with a superior ability to complex with certain APIs.

The inventors have also found that particles of inorganic solids and in particular, particles of hydrolysable silicon-containing material which themselves are smaller than the lipid particles can penetrate into the lipid particle and thereby provide a route for an API, and especially a nucleic acid, from the outside of the lipid particle into its interior. When the particles of inorganic solid are particles of hydrolysable silicon, they may optionally exist as aggregates, for example as chains. Those aggregates may extend from the exterior or the lipid particle to the interior of the lipid particle, thereby providing a route of entry into the lipid particle for an API as described further herein.

The lipid particles of the invention have been characterised as lipid particles in which, unlike regular liposomal lipid particles, the internal space of the particle remains accessible. They are termed herein as “hybrid lipid particles”, the term “hybrid” is used in recognition of both their lipidic character and components, and their non-lipid character and components (the particles of inorganic material, such as particles of silicon containing material). As such they stand in contrast to prior art lipid particles where particles of silicon-containing material are located in the interior of a lipid coating or liposome. Such prior art products tend to have the particles of silicon containing material encapsulated within the lipid. Whilst the silicon containing particles in such products may provide an advantageous environment, for example for the protection of an active ingredient or API, within the encapsulation, there is generally no route from or to the interior of the lipid encapsulation without disruption and subsequent re-forming of the encapsulation, which comes with significant disadvantages, nor is their significant exposure of the silicon containing particles on the surface of prior art particles. That means that any interaction between an active ingredient and the silicon containing particles occurs primarily within the encapsulation. The structure of hybrid lipid particles of the invention be visualised, in some embodiments, as incomplete liposomes with a route for an active compound to cross from the exterior to the interior environment.

Similar lipid particles (LPs) to those of the invention are generically disclosed in GB2210794.0 (which is incorporated herein by reference, and which may be obtained on publication or from the publicly available file on publication of other patent applications, for example international patent applications claiming priority from it). They may be manufactured by conventional lipid particle manufacturing techniques. Essentially, such techniques may be understood in broad terms as involving the preparation of a mixture of suitable lipids in a solvent, followed by the evaporation of the solvent, for example, in a rotary evaporator, to form a thin film of lipid material which is then hydrated, resulting in the formation of lipid particles, to which particles of inorganic material and an active compound (especially, an API) may be added to form a hybrid lipid particle of the invention. Although successful in producing the required product, such a manufacturing process may be resistant to effective scale-up because evaporative processes are limited by the surface area of the evaporation apparatus used, which does not increase at the same rate as volume during scale-up.

The present invention also relates to improvements in the particles disclosed in GB2210794.0, principally relating to relative dimensions of the lipid particles and the particles of inorganic material therein and the discovery that they can be made as hybrid lipid particles. The present invention also relates to improvements in the manufacturing process for those particles and also discloses advantageous properties of the particles of the invention relating to their hybrid nature as hybrid lipid particles.

Hybrid lipid particles according to the invention, also referred to as “stabilised hybrid lipid particles” (shLP) because of the use of particles of inorganic material in order to stabilise them, which in certain preferred embodiments are particles of hydrolysable silicon, resulting in lipid particles that are stabilised (especially, silicon-stabilised) hybrid lipid particles (shLP; especially, sshLP for silicon-stabilised hybrid lipid particles). Such particles are novel lipid nanoparticles (LNP) according to the invention which are especially suitable for delivery of nucleic acids. From a manufacturing perspective, the key difference between shLP and prior art LP manufacturing processes is the stage at which the active compound such as nucleic acid is incorporated into the particle. This difference is made possible by the presence of particles of inorganic material which stabilise what are essentially incompletely formed liposomes. From a product perspective, the key difference between shLPs and prior art LPs is their hybrid nature as described further herein, and also differences in relative size between the lipid particles and the particles of inorganic material. From a user perspective, the key difference between shLP and prior art LP is that shLPs may be manufactured “empty” and then loaded with an active compound or API, whereas for prior art liposomal lipid particles, the particles need to be formed “around” the active compound or API to achieve active compound/API encapsulation or else pre-formed liposomal lipid particles need to have their lipid layer disrupted using harsh conditions such as acids, solvents or detergents or elevated temperatures to allow the active compound or API to enter before the lipid layer is reformed.

Prior art processes typically use a sequential manufacturing approach where the active compound such as a nucleic acid is loaded during the initial particle formation step and the loaded LP is subsequently purified before undergoing fill and finish operations. Alternative prior art methods may involve forming “empty LPs” and then reforming the LPs around the active ingredient. In contrast, a convergent manufacturing approach is employed in accordance with the present invention for the production of shLP in which empty shLP are initially produced and purified before an active compound (especially an API, more especially a nucleic acid such as an RNA) is loaded in a separate step normally as part of the fill and finish operation. There may be considerable distance in time and space between the formation step and the loading step. This brings a number of practical advantages.

### Hybrid Lipid Particles

The use of a convergent manufacturing process in which empty hybrid lipid carrier is first produced and then the active compound (especially, API) is added to it, has advantages but also presents challenges which are mitigated in accordance with the provision of the hybrid lipid particles and method of manufacturing hybrid lipid particles of the present invention. The main challenge of the prior art is that once formed, a liposomal or other lipid carrier presents a continuous unbroken hydrophilic barrier to its internal space. This means that the active compound, especially if it is hydrophilic such as a nucleic acid, cannot easily access the internal space of the particle (i.e., it cannot be easily encapsulated within the particle). Prior art methods of allowing access to the internal space of a pre-formed liposomal lipid particles include disrupting the liposomal lipid particles, for example with a solvent or low pH or elevated temperature or using a non-convergent manufacturing process in which the liposomal carrier is initially formed around the active compound. The first method has the disadvantage that the solvent may damage the carrier or the active compound as well as introducing complexity to the process. The second method has the disadvantage that forming the liposomal carrier may necessitate the use of conditions, such as the presence of solvents, decreased pH and elevated temperatures, which may damage a fragile active compound as well as result in lack of control and lower yields because the liposomes will need to rupture and reform and some of the ruptured liposomes may not able to reform meaning that the process is not only difficult to control but also results in lowered yields and loss of lipid ingredients. Method so the present invention include methods wherein an active ingredient/API accesses the interior space of a pre-formed hybrid lipid particle without the use of a solvent, low pH or elevated temperatures to disrupt a lipid barrier to entry if the active ingredient/API. The present invention takes a different approach in that it uses hybrid lipid particles, which due to the presence of particles of inorganic material such as hydrolysable silicon and the extrusion method used in manufacture, have a largely liposomal shell which, in the absence of the active compound (especially an API), incompletely seals the interior of the particle. Furthermore, once the active compound, or a portion thereof, has accessed the interior of the particle, the particle “condenses” or “contracts” around the active compound thereby sealing in the active compound. This has been found to be especially effective if the active compound is hydrophilic, more especially if it is negatively charged, for example if the active compound is a nucleic acid. Hybrid lipid particles of the invention may optionally also include particles of inorganic solids, for example particles of hydrolysable silicon (or aggregates, such as chains thereof) which extend from the interior of the hybrid lipid particle to the interior of the hybrid lipid particle, thereby providing a route for an active compound (especially an API) from the exterior of the hybrid lipid particle to the interior of the hybrid lipid particle.

This remote loading approach of the present invention can also remove the issue of low levels of drug concentration relative to carrier which could be a further problem when the drug needs to be tested for clinical and ultimately as a product on the market.

Empty shLP according to the invention can be manufactured using any of the well-established LP manufacturing processes including evaporative, microfluidic, supercritical fluid, or flow injection techniques. Many of these techniques can be optimised to produce particles with the required particle size and low polydispersity. Where the initial particles produced are too large or their sizes are too unevenly distributed, membrane extrusion can be used to modify or re-form the particles. It has been found that the combination of the presence of particles of inorganic material (such as particles of hydrolysable silicon) which are up to half the size of the hybrid lipid particles of the invention, and the use of a manufacturing method comprising extrusion through a membrane, produces particles in accordance with the invention which are largely liposomal in character but are nonetheless hybrid particles in that their internal space is incompletely sealed-off from the surrounding medium, but which can preferably become fully sealed liposomal particles once the particles have been loaded with an active compound (especially an API). As a point of terminology, once an active compound has been added and the internal space has been fully sealed off, the lipid particles could strictly be termed liposomal lipid particles rather than hybrid lipid particles.

Once particles with the correct physical properties have been obtained these are purified and concentrated, for example by use of tangential flow filtration, before loading with an active compound such as a nucleic acid.

This convergent approach to manufacture of shLP confers a number of advantages over manufacturing process used for prior art LP products.

Firstly, manufacture of shLP is cost-effective.
Degradation of an active compound during particle formation and purification is avoided. This may be especially important if the active compound is a fragile compound such as an RNA, for example an mRNA, a saRNA or a siRNA.
Active compound loading into the shLP is performed under benign conditions; at approximately 20° C. in less than 1 hour. This is especially important if the active compound is heat sensitive, such as when it is a nucleic acid, especially an RNA, more especially single-stranded mRNA.

Secondly, the shLP manufacturing process is scalable and flexible, capable of operating with an output between a few millilitres to several thousands of litres and is therefore suitable for both low and high-demand products. Hybrid lipid particle formation techniques can be matched to the production scale required, for example using microfluidics where low volume is required and flow injection where larger volumes are needed. The process is also robust and reproducible, producing materials with consistent composition and physical properties including lipid content, particle size (e.g., low polydispersity) and zeta potential. It is cost-effective due to lower wastage of lipid compounds by elimination of a yield-reducing reforming step and by preserving the activity of an API allowing a smaller initial amount of API to be used.

Finally, shLP can increase accessibility of both high- and low-demand products.
High demand: current LP products generally require ultra-cold chain shipment and storage in order to preserve the activity of fragile APIs such as nucleic acids, whereas empty shLP can be stored and shipped under refrigerated conditions. Empty shLP can be easily shipped around the globe for loading of active compound to be performed as part of localised fill and finish operations.
Low demand: e.g. personalised medicines—small quantities of empty shLP can be supplied to clinics for loading of patient-specific active compounds, for example for loading of patient-specific nucleic acids.

Accordingly, the invention provides, as described in more detail below, a method for the manufacture of hybrid lipid particles which uses what is termed an “extrusion” technique (and preferably a method which eliminates a solvent evaporation step) wherein solvent used to prepare the mixture of lipids used and optionally used to activate the particles of inorganic material is removed from the product by a non-evaporative method after the particles have formed. Extrusion techniques involve forcing the lipid and aqueous components of a suspension of hybrid lipid particles through the pores of an extrusion membrane, often multiple times, in order to assist in the formation of a uniform multiplicity of hybrid lipid particles. The invention is based on the discovery that the inclusion of particles of inorganic material, especially hydrolysable silicon-containing particles according to the invention, in the mixture to be passed through the extrusion membrane results not only in hybrid lipid particles having similar advantageous properties to the particles described in GB2210794.0 when loaded with an active compound (especially, an API), but that the presence of the particles of inorganic material promotes the formation of the hybrid lipid particles having desirable properties.

In such extrusion methods, co-ordinating the values of the extrusion force and the lipid films' tensile strength may be especially important for the successful production of the lipid particles. In the prior art, cholesterol is known to improve the stability of the lipid film. However, a problem with cholesterol is that it makes the lipid bilayer more hydrophobic. This may change the dynamics of lipid particle formation, especially in that an insufficient amount of water may be available during lipid particle formation at the pores of the extrusion membrane, leading to premature rupture of the nascent lipid particle within the extrusion pore unless the extrusion force is increased significantly, especially when the extrusion membrane has a small pore size. Products of the present invention therefore typically utilise reduced cholesterol levels, up to and including a complete absence of cholesterol. In some embodiments, cholesterol constitutes less than 10%, less than 5% or less than 1% of the total lipid content (by weight). In other embodiments, products of the invention will contain no significant level of cholesterol.

Replacing cholesterol with particles of inorganic material (especially particles of hydrolysable silicon) resulted an unexpected outcome. The introduction of particles of inorganic material (especially particles of hydrolysable silicon) in membrane extrusion methods increased the amount of water available during lipid particle formation at the pores of the extrusion membrane. Hence, lipid particle formation at the pore opening shows no sign of premature rupture of the nascent lipid particle within the extrusion pore.

Such a discovery is surprising because one might assume that in order for the particles of inorganic material (for example particles of hydrolysable silicon) to have a beneficial effect on the stability and performance of the hybrid lipid particles, relatively high amounts of particles of inorganic material (for example particles of hydrolysable silicon) would need to remain as part of the lipid particle. That is what is found when lipid particles are prepared by conventional evaporative methods as described in GB2210794.0. Surprisingly, that is not the case when hybrid lipid particles are prepared using an extrusion technique in accordance with the invention. The use of an extrusion technique results in a significant reduction in the level of particles of inorganic material that remain as part of the lipid particle and it has been found that if a relatively high level of particles of inorganic material is used in production of hybrid lipid particles by an extrusion technique, the level of particles of inorganic material in the final hybrid lipid particle product can be allowed to drop to a relatively low level during extrusion and yet still exhibit advantageous properties normally expected of lipid particles prepared in an “evaporative” method wherein relatively high levels of particles of inorganic material must be retained in the final lipid particles for the advantages to ensue. Accordingly, the invention also relates to hybrid lipid particles having relative low levels of particles of inorganic material which have been prepared by an extrusion technique involving relatively high levels of particles of inorganic material. Corresponding methods are also encompassed by the invention.

### Theoretical Basis

The Applicant does not wish to be bound by any particular theoretical explanation underlying the invention. Merely in the interests of promoting understanding, two non-mutually exclusive mechanisms which could serve to underpin the Inventors' unexpected finding that hybrid lipid particles having relative low levels of particles of inorganic material have beneficial properties if they are manufactured by a method using relatively high levels of particles of inorganic material which are then reduced to relatively low levels by an extrusion technique.

The first theoretical explanation is that what matters most for the beneficial properties of the hybrid lipid particles is that the particles of inorganic material are present in specific niches or locations in or on the hybrid lipid particle where they are especially-strongly associated with the lipid molecules. It is thought that hydrogen bonding in this scenario decreases the barrier for translocation of an active compound (especially an API), i.e. increases the permeability of the lipid particle to the active compound. An initially relatively high number of particles of inorganic material may be required to ensure that all or most of these specific niches are filled, however, once they are filled, the overall levels of inorganic particles can be allowed to fall without detriment.

An alternative or additional explanation may be that the inorganic material (for example, hydrolysable silicon) may need to be at initially relatively high levels to assist with dehydrating hybrid lipid particles (i.e. consuming water molecules trapped in the hybrid structure by a silicon hydrolysis reaction) and that once this initial dehydration has taken place, high levels of inorganic material are no longer required.

### SUMMARY OF THE INVENTION

According to a first aspect of the invention, there is provided a method of manufacturing a suspension of hybrid lipid particles comprising the steps of:
A. mixing one or more lipids in a solvent or solvent mixture and a suspension of particles of inorganic material in a solvent or solvent mixture, into an aqueous medium; then
B. passing the mixture resulting from Step A through the pores of an extrusion membrane,
   wherein the hybrid lipid particles have a mean diameter of at least two times larger than the mean diameter of particles of inorganic material.

According to a second aspect of the invention there is provided an aqueous suspension of hybrid lipid particles having a mean diameter of between 50 and 150 nm. The hybrid lipid particles preferably comprise one or more lipids and particles of inorganic material, wherein the mean diameter of the hybrid lipid particles is at least two times the mean diameter of the particles of inorganic material.

According to a third aspect of the invention there is provided an aqueous suspension of liposomal lipid particles having a mean diameter of between 50 and 150 nm. The liposomal lipid particles comprise an active compound (especially an API), one or more lipids and particles of inorganic material, wherein the mean diameter of the hybrid lipid particles is at least two times the mean diameter of the particles of inorganic material.

According to a fourth aspect of the invention there is provided a method of manufacturing an aqueous suspension of liposomal lipid particles according to the third aspect of the invention from an aqueous suspension of hybrid lipid particles according to the second aspect of the invention comprising the step of contacting the aqueous suspension of hybrid lipid particles with an active compound (especially, an API).

According to a fifth aspect of the invention there is provided a lyophilized powder of liposomal lipid particles having a mean diameter of between 50 and 150 nm, the liposomal lipid particles comprising a mixture of one or more cationic lipids or ionisable lipids with one or more further lipids selected from neutral lipids and polar lipids, and optionally one or more additional lipid components, the liposomal lipid particles comprising particles of inorganic material (preferably particles of hydrolysable silicon) having a mean diameter of up to one half of the mean diameter of the liposomal lipid particles, wherein the weight ratio of the particles of inorganic material to lipid is between 1:2 and 1:100 (preferably between 1:10 and 1:100, more preferably between 1:20 and 1:10) and wherein the liposomal lipid particles further comprise one or more active compounds, for example one or more active pharmaceutical ingredients (APIs), at least a portion of which are encapsulated within the interior of the liposomal particle.

According to a sixth aspect of the invention there is provided a lyophilized powder of hybrid lipid particles having a mean diameter of between 50 and 150 nm, the hybrid lipid particles comprising a mixture of one or more cationic lipids or ionisable lipids with one or more further lipids selected from neutral lipids and polar lipids, and optionally one or more additional lipid components, the hybrid lipid particles comprising particles of inorganic material (preferably particles of hydrolysable silicon) having a mean diameter of up to one half of the mean diameter of the hybrid lipid particles, wherein the weight ratio of the particles of inorganic material to lipid is between 1:2 and 1:100 (preferably between 1:10 and 1:100, more preferably between 1:20 and 1:10).

According to a seventh aspect of the invention there is provided a pharmaceutical composition comprising an aqueous suspension of hybrid lipid particles of the invention or a lyophilized powder of hybrid lipid particles of the invention, or an aqueous suspension of liposomal lipid particles of the invention or a lyophilized powder of liposomal lipid particles of the invention.

According to further aspects of the invention there is provided the use of a pharmaceutical composition of the invention as a medicament, and related methods of medical treatment. The invention also provides an aqueous suspension of lipid particles comprising a pharmaceutically active ingredient (API), produced by a method comprising:
(a) obtaining an aqueous suspension of hybrid lipid particles manufactured by:
   (i) mixing one or more lipids in a solvent or solvent mixture and a suspension of particles of inorganic material in a solvent or solvent mixture, into an aqueous medium;
   (ii) passing the mixture resulting from Step (i) through the pores of an extrusion membrane; and
   (iii) optionally purifying and/or sterilizing the suspension by tangential flow filtration, and
(b) contacting the hybrid lipid particles with an active compound, especially a pharmaceutically active ingredient (API).

The invention also provides an aqueous suspension of hybrid lipid particles comprising a pharmaceutically active ingredient (API), produced by a method comprising:
(a) obtaining an aqueous suspension of hybrid lipid particles manufactured by:
   (i) mixing one or more lipids in a solvent or solvent mixture and a suspension of particles of inorganic material in a solvent or solvent mixture, into an aqueous medium;
   (ii) passing the mixture resulting from Step (i) through the pores of an extrusion membrane; and
(b) purifying and/or sterilizing the suspension by tangential flow filtration.

### BRIEF DESCRIPTION OF THE DRAWINGS

FIG. 1A is a diagrammatic representation of a manufacturing method of certain embodiments of the present invention.

FIG. 1B shows diagrammatically the manufacturing method in more detail according to specific embodiments.

FIG. 2 shows an overview of the manufacturing strategy for the generation of a scalable process for the production of micellar lipid particles.

FIG. 3 shows the manufacturing process of the micellar lipid particles by an evaporative process.

FIG. 4 shows the results of a visual investigation of the obtained solutions for sample MVI0001.

FIG. 5 shows DLS and Zeta potential analysis of sample MVI0001 over extrusion steps.

FIG. 6 shows the general procedure for the generation of sample MVI0002.

FIG. 7 shows the results of visual investigation of the obtained solution for sample MVI0002.

FIG. 8 shows DLS and Zeta potential analysis of sample MVI0002 over the extrusion steps.

FIG. 9 shows the general procedure for generation of sample MVI0003.

FIG. 10 shows results of visual investigation of the obtained solutions for sample MVI0003.

FIG. 11 shows DLS and Zeta potential analysis of sample MVI0003 over the extrusion steps.

FIG. 12 shows results of visual investigation of the obtained solutions for sample MVI0004.

FIG. 13 shows DLS and Zeta potential analysis of sample MVI0004 over the extrusion steps.

FIG. 14 shows representative pictures for visual investigation of the obtained solutions for sample MVI0005.

FIG. 15 shows DLS and Zeta potential analysis of sample MVI0005 over the extrusion steps.

FIG. 16 shows DLS and Zeta potential analysis of sample MVI0006 over the extrusion steps.

FIG. 17 shows the experimental setup for sample MVI0007 using the 0.8 μm membrane.

FIG. 18 shows the results of visual investigation of the solution obtained at each step for experiment MVI0007.

FIG. 19A shows pictures of the 0.4 μm and 0.1 μm membranes after extrusion without the use of a 0.8 μm membrane.

FIG. 19B shows pictures of the 0.8 μm, 0.4 μm and 0.1 μm membranes after extrusion.

FIG. 20 shows DLC and Zeta potential analysis of MVI0007 over the extrusion steps.

FIG. 21 shows results of investigation of the dilution effect on DLS and Zeta potential measurements (FIG. 21A: 20× dilution factor; FIG. 21B: 5× dilution factor).

FIG. 22 shows results of visual investigation of the solution obtained at each step for experiment MVI0008.

FIG. 23 shows DLS and Zeta potential analysis of MVI0008 over the extrusion steps.

FIG. 24 shows the experimental setup for MVI0011.

FIG. 25 shows the result of visual investigation of the solution obtained at each step for experiment MVI0011.

FIG. 26 shows DLS and Zeta potential investigation of MVI001.

FIG. 27 shows the result of visual investigation of the solution obtained at each step for experiment MVI0012.

FIG. 28 shows membranes after extrusion.

FIG. 29 shows DLS and Zeta potential investigation of MVI0012.

FIG. 30 shows pressure during extrusion in experiment MVI0007 with extruded volume 50 ml using 25 mm diameter membranes.

FIG. 31 shows pressure during extrusion in experiment MVI0011 with extruded volume 100 ml using 25 mm diameter membranes. SiNP solution was filtered before extrusion with 0.8 μm syringe filter.

FIG. 32 shows pressure during extrusion in experiment MVI0012 with extruded volume 1 L using 47 mm diameter membrane.

FIG. 33 shows chromatograms of the separate lipid components.

FIG. 34 shows a chromatogram of the lipid mix.

FIG. 35 shows the gradient profile for modification and initial HPLC methods.

FIG. 36 shows a chromatogram of the lipid mix at final product concentration of [DOTAP-Cl]=0.725 mg/ml, [DOPE]=0.73 mg/ml and [mPEG2000′DSPE]=0.145 mg/ml.

FIG. 37 shows calibration curves for each lipid for MVI0010.

FIG. 38 shows a chromatogram of experiment MVI0010.

FIG. 39 shows calibration curves for experiment MVI0017.

FIG. 40 shows measured chromatograms for undiluted samples MVI0017.

FIG. 41 shows calibration curves for MVI0017 using diluted standard lipid solutions.

FIG. 42 shows measured chromatograms for 2× diluted samples MVI0017.

FIG. 43 shows chromatograms of nuclease free water and THR-GLY solutions.

FIG. 44 shows chromatograms at different time point for a sample stored at RT.

FIG. 45 shows concentration of the lipids over time in the analysed sample at RT.

FIG. 46 shows chromatograms at different time point for sample stored at RT.

FIG. 47 shows concentration of the lipids over time in the analysed sample at 4° C.

FIG. 48 shows TFF setup used in the examples.

FIG. 49 shows a MeOH calibration curve.

FIG. 50 shows 1H-NMR signals for MVI0010 pre-TFF and post-TFF.

FIG. 51 shows experimental setup for MVI0013 using the prefiltration step.

FIG. 52 shows the result of visual investigation of the solution obtained at each step for experiment MVI0013.

FIG. 53 shows pictures of the 0.8 μm hydrophilic polyethersulfone syringe filter (A) and of the extrusion membranes (B) after filtration and extrusion respectively.

FIG. 54 shows DLC and Zeta potential analysis of MVI0013 over the extrusion steps.

FIG. 55 shows extrusion pressure during investigation of experiment MVI0013.

FIG. 56 shows Zeta potential analysis of MVI0013.

FIG. 57 shows experimental setup for MVI0014.

FIG. 58 shows results of visual investigation of the solution obtained at each step for experiment MVI0014.

FIG. 59 shows DLS and Zeta potential analysis of MVI0014 over the extrusion steps.

FIG. 60 shows the results of the extrusion pressure investigations on MVI0014.

FIG. 61 shows the experimental setup for MVI0015.

FIG. 62 shows the results of visual investigation of the solution obtained at each step for experiment MVI0015.

FIG. 63 shows DLS and Zeta potential analysis of MVI0015 over extrusion steps.

FIG. 64 shows the experimental procedure used to produce a 2 kg batch.

FIG. 65 shows pictures of the extrusion membranes after the extrusion steps.

FIG. 66 shows results of DLS and Zeta potential investigation of MVI0022.

FIG. 67 shows a trend of size increase with extrusion volumes.

FIG. 68 shows various possible configurations for rapid mixing in accordance with certain embodiments of the invention.

FIG. 69 shows long term stability of hybrid lipid particles of the invention compared with lipid particles formulated without silicon or other particles of inorganic material.

FIG. 70 shows size stability of particles of the invention is maintained after loading with mRNA.

FIGS. 71 to 81 show methods and results relating to examples 17 and 18.

FIG. 71 shows (A) Workflow of the original lipid thin film hydration method (Method 1) where organic solvent is evaporated from activated silicon nanoparticles, and the revised protocol (Method 2) where the activated silicon nanoparticle suspension is added directly to the aqueous phase. (B) More sedimentation was observed with Method 2 (lower image) after lipid film hydration. (C) DLS results indicated lower PDI and higher zeta potential for hybrid lipid particles produced using the revised method.

FIG. 72 shows diagrammatically removing the second evaporation step and optimizing a solvent injection mixing method. (A) Workflow of the initial direct injection mixing approach (Method 3). (B) Less sedimentation was seen compared with lipid thin film hydration. (C) Modified workflows incorporating an additional 0.8 μm extrusion step and other adjustments as indicated. (D) The additional extrusion step (Method 4) led to smaller hybrid lipid particles with similar PDI and zeta potential. (E) Prefiltration before extrusion (Method 6, right) removed insoluble aggregates that would otherwise accumulate on the 0.8 μm extrusion membrane (Method 4, left), although the 0.4 μm membrane appeared to remove additional aggregated material. (F) Introducing the prefiltration step did not impact the properties of the final hybrid lipid particles.

FIG. 73 shows purification of hybrid lipid particles of the invention by TFF. (A) Schematic of the experimental setup illustrating how a second pump (Pump 2) was included to enable both ultrafiltration and diafiltration operations. (B) Representative result illustrating successful removal of MeOH from hybrid lipid particles produced via Method 5, as judged by ¹H NMR. (C, D) The TFF process was found to slightly modify the DLS characteristics (C) and lipid content (D) of the particles, but these variations lie within relevant reference ranges and are therefore not considered problematic.

FIG. 74 shows Initial large-scale (1 L) run to produce hybrid lipid particles. (A) Schematic of the workflow used; a scale-up of Method 5. (B) Representative images of extrusion membranes and sample aliquots (lower-right quadrant), showing buildup of aggregates despite the use of larger membranes as well as some remaining insoluble material after the first extrusion step. (C) Accumulation of aggregates on the 0.8 μm and 0.4 μm membranes resulted in substantially higher operating pressure than on a 50 mL scale (Method 4). (D) DLS characteristics of the hybrid lipid particles were not adversely affected and are within reference ranges.

FIG. 75 shows production of a 2 L batch of hybrid lipid particles. (A) Schematic of the workflow used. Note that the TFF step contained two stages: ultrafiltration to concentrate the sample from 4 L to 2 L, followed by diafiltration to remove MeOH and free lipids. (B) The extrusion steps were compromised by significant buildup of aggregates on the membranes, compromising their performance. (C) For the finished batch, average size and PDI were outside the reference ranges. (D) This was due to deterioration of the extrusion membranes with excessive accumulation of aggregates, indicating the need for a prefiltration step after activation of hybrid lipid particles when manufacturing hybrid lipid particles at scale.

FIG. 76 illustrates the effects of prefiltering the activated silicon nanoparticle suspension improves the process. (A) Schematic of the modified workflow, designated Method 7. (B) In this case, there was no significant accumulation of aggregates on the extrusion membranes. (C-E) This was reflected in considerably lower operating pressures during extrusion, compared with the protocol without prefiltration (C), and led to sshLNPs with DLS properties that were well within reference ranges, both on a small scale (D) and for a 1 L batch size (E).

FIG. 77 illustrates that hybrid lipid particles produced at small scale (using lipid thin film hydration, Method 2) and large scale (using Method 7) exhibit similar physical and functional properties. (A) The large-scale process produced slightly smaller final particles with comparable PDI. (B) Zeta potential was unaffected by scale-up. (C) Both methods gave hybrid lipid particles with equivalent RNA encapsulation efficiency. (D) Large-scale manufacture also maintained transfection efficiency of HEK293 cells with an mRNA encoding firefly luciferase (fLuc). Luminescence was measured to determine fLuc expression levels at 24 h post-transfection. Lipofectamine 2000 was used as positive control and untreated cells as negative control. Panels (A-C) report mean±SD for samples analysed in triplicate, while panel D shows mean±SD for three independent biological replicates.

FIG. 78 shows that inverse mixing after silicon nanoparticle activation does not compromise properties of the hybrid lipid particles. (A) Workflow of Methods 4 and 5, indicating the sole difference in mixing order. (B) Comparison of DLS-derived parameters for samples prepared via Methods 4 and 5. Although the final zeta potential was lower in this the run (right-hand graph), subsequent experiments showed Method 5 can reliably produce hybrid lipid particles with zeta potential within the reference range, including on a large scale.

FIG. 79 shows that introducing a prefiltration step before extrusion (Method 6) significantly reduced operating pressure on the 0.8 μm and 0.4 μm extrusion membranes, compared to the method without prefiltration (Method 4).

FIG. 80 illustrates the development of an HPLC-based assay for lipid recovery. (A) Chromatogram of a mixed lipid standard solution with the DOTAP, DOPE, and mPEG2000-DSPE components present at the same molar ratio used to prepare hybrid lipid particles. (B) Calibration curves for lipid reference solutions used in the assay for Method 5. (C) Chromatogram of the post-extrusion sample produced using Method 5, showing the presence of two unidentified impurity peaks. In this sample, the observed values for lipid recovery were 86%, 81%, and 76%, respectively, for DOTAP, DOPE, and mPEG2000-DSPE. (D) An alternative UPLC assay is now in development, to achieve improved peak shape for the PEGylated lipid in particular. The chromatogram shows a representative result for the mixed lipid standard solution.

FIG. 81(A) demonstrates increasing the membrane surface area available for extrusion, by using two sets of extrusion membranes in parallel.

FIG. 81(B) shows that in combination with prefiltration of the activated silicon nanoparticles, this prevented buildup of residue on the 0.8 μm and 0.4 μm membranes. A yellow coloration of the 0.1 μm membranes was seen, suggesting removal of smaller aggregates and/or oversized particles from the sample.

FIG. 82 is a transmission electron microscope (TEM) image of hybrid lipid particles according to an embodiment of the invention. It shows the aggregation of silicon particles, as described herein. The particle labelled “lipid” is a hybrid lipid particle according to embodiment so the invention. The image also shows relatively smaller silicon particles which are part if an aggregation which extends from outside the hybrid lipid particle to inside the hybrid lipid particle.

### DETAILED DESCRIPTION OF INVENTION

According to a first aspect of the invention, there is provided a method of manufacturing a suspension of hybrid lipid particles comprising the steps of:
A. mixing one or more lipids in a solvent or solvent mixture and a suspension of particles of inorganic material in a solvent or solvent mixture, into an aqueous medium; then
B. passing the mixture resulting from Step A through the pores of an extrusion membrane,
   wherein the hybrid lipid particles have a mean diameter of at least two times larger than the mean diameter of particles of inorganic material.

According to a second aspect of the invention there is provided an aqueous suspension of hybrid lipid particles having a mean diameter of between 50 and 150 nm. The hybrid lipid particles comprise one or more lipids and particles of inorganic material, wherein the mean diameter of the hybrid lipid particles is at least two times the mean diameter of the particles of inorganic material.

According to a third aspect of the invention there is provided an aqueous suspension of liposomal lipid particles having a mean diameter of between 50 and 150 nm. The liposomal lipid particles comprise an active compound (especially an API), one or more lipids and particles of inorganic material, wherein the mean diameter of the hybrid lipid particles is at least two times the mean diameter of the particles of inorganic material.

According to a fourth aspect of the invention there is provided a method of manufacturing an aqueous suspension of liposomal lipid particles according to the third aspect of the invention from an aqueous suspension of hybrid lipid particles according to the second aspect of the invention comprising the step of contacting the aqueous suspension of hybrid lipid particles with an active compound (especially, an API).

According to a fifth aspect of the invention there is provided a lyophilized powder of liposomal lipid particles having a mean diameter of between 50 and 150 nm, the liposomal lipid particles comprising a mixture of one or more cationic lipids or ionisable lipids with one or more further lipids selected from neutral lipids and polar lipids, and optionally one or more additional lipid components, the liposomal lipid particles comprising particles of inorganic material (preferably particles of hydrolysable silicon) having a mean diameter of up to one half of the mean diameter of the liposomal lipid particles, wherein the weight ratio of the particles of inorganic material to lipid is between 1:2 and 1:100 (preferably between 1:10 and 1:100, more preferably between 1:20 and 1:10) and wherein the liposomal lipid particles further comprise one or more active compounds, for example one or more active pharmaceutical ingredients (APIs), at least a portion of which are encapsulated within the interior of the liposomal particle.

According to a sixth aspect of the invention there is provided a lyophilized powder of hybrid lipid particles having a mean diameter of between 50 and 150 nm, the hybrid lipid particles comprising a mixture of one or more cationic lipids or ionisable lipids with one or more further lipids selected from neutral lipids and polar lipids, and optionally one or more additional lipid components, the hybrid lipid particles comprising particles of inorganic material (preferably particles of hydrolysable silicon) having a mean diameter of up to one half of the mean diameter of the hybrid lipid particles, wherein the weight ratio of the particles of inorganic material to lipid is between 1:2 and 1:100 (preferably between 1:10 and 1:100, more preferably between 1:20 and 1:10).

According to a seventh aspect of the invention there is provided a pharmaceutical composition comprising an aqueous suspension of hybrid lipid particles of the invention or a lyophilized powder of hybrid lipid particles of the invention, or

an aqueous suspension of liposomal lipid particles of the invention or a lyophilized powder of liposomal lipid particles of the invention.

According to further aspects of the invention there is provided the use of a pharmaceutical composition of the invention as a medicament, and related methods of medical treatment.

The invention also provides an aqueous suspension of lipid particles comprising a pharmaceutically active ingredient (API), produced by a method comprising:
(b) obtaining an aqueous suspension of hybrid lipid particles manufactured by:
   (i) mixing one or more lipids in a solvent or solvent mixture and a suspension of particles of inorganic material in a solvent or solvent mixture, into an aqueous medium;
   (ii) passing the mixture resulting from Step (i) through the pores of an extrusion membrane; and
   (iii) optionally purifying and/or sterilizing the suspension by tangential flow filtration, and
(b) contacting the hybrid lipid particles with an active compound, especially a pharmaceutically active ingredient (API).

The invention also provides an aqueous suspension of hybrid lipid particles comprising a pharmaceutically active ingredient (API), produced by a method comprising:
(c) obtaining an aqueous suspension of hybrid lipid particles manufactured by:
   (i) mixing one or more lipids in a solvent or solvent mixture and a suspension of particles of inorganic material in a solvent or solvent mixture, into an aqueous medium;
   (ii) passing the mixture resulting from Step (i) through the pores of an extrusion membrane; and
(d) purifying and/or sterilizing the suspension by tangential flow filtration.

### Particles of Inorganic Material

According to all aspects of the invention, the particles of inorganic material may be of any inorganic material including mixtures of one or more inorganic materials. In preferred embodiments of all aspects of the invention the particles of inorganic material are particles of or comprising hydrolysable silicon. Preferably such particles comprising hydrolysable silicon consist of at least 70%, at least 80%, at least 90%, most preferably at least 98% hydrolysable silicon (which are collectively referred to as “hydrolysable silicon particles” or, interchangeably, “particles of hydrolysable silicon”).

### Solvent and Activating Solvent, and Mixtures Thereof

The particles of inorganic material are, according to methods of the invention, may optionally be activated by exposing them to a solvent or a solvent mixture. Preferably the solvent or solvent mixture is an activating solvent or an activating solvent mixture. An activating solvent or an activating solvent mixture may be understood as a solvent or solvent mixture which “activates” the particles of inorganic material (preferably hydrolysable silicon particles). Activating is to be understood as including the washing of contaminants from the particles so that the particles are able to fully interact with other ingredients present in the method or product of the invention. “Activating” may also include removal of contaminants from any pores present in the particles. Contaminants include soluble contaminates such as surface oxides and hydroxides, and solid contaminants such as surface “dust”. Preferably that solvent is an organic compound for example a volatile organic compound, such as an alcohol. Preferably the activating solvent mixture comprises methanol. More preferably, the activating solvent is methanol. The activating solvent may optionally be allowed to evaporate from the particles of inorganic material before the method of the invention proceeds to subsequent steps. Alternatively, the activating solvent is not pre-evaporated. According to certain embodiments the activating solvent is toxic and/or not recognised as an approved pharmaceutical ingredient. It is to be understood that the solvent or solvent mixture in which the one or more lipids are supplied may be the same solvent or solvent mixture or a different solvent or solvent mixture to the solvent or solvent mixture in which the particles of inorganic material are suspended.

According to certain preferred embodiment of all aspects of the invention, “activation” by a solvent or a solvent mixture comprises surface treatment of particles of inorganic material which are particles of or particles comprising hydrolysable silicon. Surface treatment with a solvent or solvent mixture is to be understood as comprising surface treatment with one or more alcohols. Especially preferred alcohols include methanol, benzyl alcohol and methanol (or mixture thereof). “Surface treatment” of hydrolysable silicon may optionally comprise formation of Si—O(CH₂)ₓCH₃ (where x is zero for methanol, 1 for ethanol, etc), Si—(CH₂)ₓCH₃ and/or Si—H moieties on the particles' surface, where —(CH₂)ₓCH₃ may be replaced by other carbon-containing groups, such as an aromatic ring, for example when the alcohol is or comprises benzyl alcohol).

### Hydrolysable Silicon

“Hydrolysable silicon” as used herein encompasses pure elemental silicon. However, complete purity is not required. Conversely, the invention is not intended to cover pure silica (including sand, quartz, silica gel). A key requirement is that the material is hydrolysable, that is to say, that it will tend to breakdown under physiological conditions to soluble products such as orthosilicic acid (OSA). According to certain embodiments the definition “hydrolysable silicon” is met if at least half of the mass of the material is hydrolysed to soluble products within a month of injection into a subject (for example following intramuscular or subcutaneous injection).

Hydrolysable silicon according to certain embodiments of the invention is preferably mesoporous. This is to say, it contains pores of between 2 to 50 nm in diameter.

Particles of hydrolysable silicon may be purchased commercially or may be produced by any suitable method.

In certain embodiments, the particles comprising hydrolysable silicon may be pure or substantially pure silicon.

The particles may alternatively be another hydrolysable silicon-containing material. If the particles are not pure silicon, they comprise at least about 50% by weight silicon, i.e. they comprise at least about 50% by weight silicon atoms, based on the total mass of atoms in the particles. For example, the silicon particles may contain at least about 60, about 70, about 80, about 90 or about 95% by weight silicon. The particles may show a rate of hydrolysis, for example in PBS buffer at room temperature, of at least 10% of the rate of hydrolysis of pure silicon particles of the same dimensions. Assays for hydrolysis of silicon-containing material are widely known in the art; see, for example, WO 2011/001456, incorporated herein by reference in its entirety.

Although the particles may contain traces of silica, silica is not hydrolysable silicon. At least about half of the silicon atoms in the particles may be in the form of elemental silicon (or doped elemental silicon).

The particles may, especially, be nanoparticles. The nanoparticles according to certain embodiments may have a nominal diameter in a range of about 1 to about 500 nm, especially about 1 to about 250 nm, more especially about 1 to about 100 nm (e.g., about 30 nm). As used herein, the term “nominal diameter” may refer to the mean diameter and at least about 90% of total mass of particles in a sample of particles may fall within the size range specified.

The particles may be porous, especially mesoporous. Particles comprising hydrolysable doped silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement. If the particles are porous, their total surface area will be increased by virtue of their porosity. For example, their surface area may be increased by at least about 50% or at least about 100%, compared to the surface area of a corresponding non-porous particle. In many circumstances, porous particles will in reality have a much greater increase in total surface area by virtue of their porosity. According to certain embodiments the porosity is at least about 30, about 40, about 50 or about 60%; meaning that, respectively, at least about 30, about 40, about 50 or about 60% of the particle volume is pore space. Pore diameters may be in a range of from about 1 nm to about 50 nm, for example from about 1 nm to about 5 nm.

### Doped Silicon

It is preferred that the hydrolysable silicon for use in all aspects of the invention comprises (or consists of) hydrolysable doped silicon.

As used herein, the term “doped silicon” may refer to silicon which behaves as an extrinsic semiconductor due to the presence of dopant atoms, whether the dopant atoms are substitutional (taking the place of Si atoms) or interstitial (amongst, not displacing, Si atoms).

Advantageously, the silicon particles are doped at a level of at least about 1×10¹⁵, most especially at least about 1×10¹⁶ dopant atoms per cm³.

For example, the particles may be doped at a level of at least about 1×10¹⁷, at least about 1×10¹⁸, or at least about 1×10¹⁹ dopant atoms per cm³.

The silicon particles may be doped at a level of up to 1×10²⁰ dopant atoms per cm³.

The silicon particles may be n-doped or p-doped. The silicon particles may be doped with one or more elements selected from B, P, Mg, Cu, Ga, Al, In, Bi, Ge, Li, Xe, N, Au and Pt. Thus, the dopant may be a p-dopant, especially boron. The dopant may be an n-dopant, especially phosphorus.

When boron is used as the dopant, as is preferred, doping levels of 1×10⁵ dopant atoms per cm³, and 1×10²⁰ dopant atoms per cm³ correspond, respectively, to a resistivity of 13.6 Ω-cm, and 1.3 m Ω-cm. Embodiments wherein boron is the dopant do not exclude silicon which, while doped (e.g., heavily doped) with boron, is additionally doped with other elements (preferably, in such cases, the majority dopant is boron).

As used herein, the term “heavy doping” is understood to mean doping of at least about 1×10¹⁵ dopant atoms per cm³. In some preferred embodiments, dopant is present at levels of at least about 1×10¹⁶ dopant atoms per cm³. Thus, in particularly preferred embodiments, the dopant is boron which is present at levels of at least about 1×10¹⁶ boron atoms per cm³. For example, there may be boron present at levels of at least about 1×10¹⁶ boron atoms per cm³ and up to about 1×10²⁰ boron atoms per cm³.

Where silicon is referred to herein as “undoped” (such as the particles of composition SIS0012 of Examples 1 and 2), it may mean that no or only small amounts of dopant atoms are present, for example at most about 1×10² dopant atoms per cm³. Additionally, or alternatively, “undoped” silicon may mean silicon that does not behave as an extrinsic semiconductor.

Doping the silicon particles may improve their various functions as described herein, especially their ability to make the interior of a hybrid lipid particle according to the invention accessible. Additionally, or alternatively, doping the silicon particles can improve the stability of an active compound (especially an API) in the interior of a liposomal particle according to the invention.

The semiconductor industry provides a ready source of appropriately doped silicon and a wealth of expertise in silicon doping techniques. The manufacture of doped silicon is well-understood in the semiconductor industry and includes ion implantation and diffusion methods, making doped silicon per se readily available. As an example of a diffusion method, silicon powder and a doping reagent (for example B₂O₃ for boron doping) are mixed under N₂ atmosphere at a temperature of 1050° C.-1175° C. for a few minutes, to allow the dopant (such, for example, as boron) to diffuse into the silicon.

### Manufacturing Particles of Inorganic Material

It will be appreciated that the particles may be produced by various techniques familiar to the skilled person.

The techniques may include, for example, purely physical (sometimes referred to, in the art, as “non-wet”) processes having bulk inorganic solid material (especially, silicon wafer) as the starting material, such as pulsed laser ablation, thermal degradation and ball milling. Thus, the particles may be obtainable by a method comprising or consisting of one or more of pulsed laser ablation, thermal degradation, and ball milling, of bulk inorganic material (especially, silicon wafer).

Additionally or alternatively, the particles may be produced by chemical (sometimes referred to, in the art, as “wet”) techniques, including but not limited to electrochemical etching of bulk inorganic material (especially, silicon wafer). Such techniques optionally include HF etching as described above. Thus, the particles may be obtainable by a method comprising or consisting of electrochemical etching of bulk inorganic material (especially, silicon wafer).

Once formed, the particle of inorganic material (for example particles of hydrolysable silicon) may be sorted by size, such, for example, by air classification, sieving and/or filtration. Thus, the particles may be obtainable by a method comprising one or more of air classification, sieving and/or filtration.

Thus, for example, the particles may be obtainable by a method comprising producing particles of inorganic solid material from solid material, especially from silicon wafer, such as by one or more of pulsed laser ablation, thermal degradation, ball milling, and electrochemical etching, of bulk inorganic material (especially, silicon wafer); followed, optionally, by sorting by size, such as by air classification, sieving and/or filtration.

Optionally, the particles may be washed before use, such as in methanol or ethanol. In the art, this may be termed “activation” and is discussed elsewhere herein.

The particles of inorganic solid material thus obtained may have a narrow size distribution and homogeneous surface chemistry, leading to batch-to-batch reliability and reproducibility of one or more of the advantages described herein.

Suitable physical and chemical techniques are set out, for example, in WO 2011/012867 A1 (in the name of SISAF LTD); in Tokarska K et al., Facile production of ultra-fine silicon nanoparticles, R. Soc. Open Sci., 2020, 7: 200736; and in Kim, T., Lee, J. Silicon nanoparticles: fabrication, characterization, application and perspectives, Micro and Nano Syst. Lett, 2023, 11: 18, each of which is incorporated herein by reference in its entirety.

### Dimensions of Hybrid Lipid Particles and Liposomal Particles

According to all aspects of the invention, the hybrid lipid particles of the invention and the liposomal lipid particles of the invention have a mean diameter of between 50 nm and 400 nm, for example between 50 nm and 200 nm, for example between 60 nm and 150 nm, for example between 60 nm and 120 nm, for example between 60 nm and 100 nm. Particles which have a mean diameter between 1 nm and 100 nm may be referred to as nanoparticles (NPs) or lipid nanoparticles (LNPs). Hybrid lipid particles of the invention which are silicon stabilized hybrid lipid nanoparticles, may be referred to as sshLNPs. According to certain preferred embodiments using particles of hydrolysable silicon, the hybrid lipid particles have a mean diameter of between 50 nm and 200 nm (for example between 70 nm and 160 nm) and the particles of hydrolysable silicon have a mean diameter of between 4 nm and 20 nm (for example between 6 nm and 16 nm).

### Hybrid Lipid Particles

Hybrid lipid particles of the invention include micelles, incomplete micelles, liposomes, incomplete liposomes, and (e.g., solid or semi-solid) lipid globules.

Hybrid lipid particles according to the invention may, in some embodiments comprise a lipid bilayer surrounding an aqueous interior space (or in lyophilised form, an empty interior space). However, rather than that lipid bilayer completely sealing off the interior space from the environment external to the particle, the interior space is at least partially accessible. It may be accessed by an active compound, especially a hydrophilic compound which otherwise would have difficulty crossing the lipid bilayer, especially a negatively charged nucleic acid, a representative example being mRNA. This accessibility maybe achieved by a discontinuity in the lipid bilayer or by a route through the lipid bilayer provided by the presence of particles of inorganic material (for example hydrolysable silicon particles) penetrating through the lipid bilayer.

### Liposomal Lipid Particles

Liposomal lipid particles according to the invention comprise a lipid bilayer surrounding an aqueous interior space. In contrast to the hybrid lipid particles of the invention, the lipid bilayer completely seals off the interior space from the environment external to the particle, thereby protecting whatever material is held within the interior space, for example protecting an API held within the interior space.

### Transition Between Hybrid Lipid Particles and Liposomal Lipid Particles

It is surprising that the hybrid lipid particles of the invention are stable for extended periods of time. One might expect the incompletely-sealed lipid bilayer to seal up in storage effectively transforming the hybrid lipid particles of the invention to liposomal particles. That is not the case. It has been found that in the absence of an active compound or API, hybrid lipid particles of the invention are stable for a long time. For example, according to some embodiments such stability may be defined as the interior space of the particles remaining accessible rather than sealed off from the outside environment. According to certain embodiments such stability may manifest in the interior space of at least 90% of the hybrid lipid particles remaining accessible from the outside environment for at least 1 week, at least 2 weeks or at least 4 weeks in aqueous suspension at room temperature (understood as 25° C. throughout this specification). According to other embodiments such stability may manifest in the interior space of at least 90% of the hybrid lipid particles remaining accessible from the outside environment for at least 4 weeks, at least 8 weeks, at least 16 weeks or at least 32 weeks in aqueous suspension at 4° C. According to other embodiments such stability may manifest in the interior space of at least 90% of the hybrid lipid particles remaining accessible from the outside environment for at least 4 weeks, at least 8 weeks, at least 16 weeks, at least 32 weeks, or at least 64 weeks in aqueous suspension at −20° C. According to certain embodiments such stability may manifest in the interior space of at least 90% of the hybrid lipid particles remaining accessible from the outside environment for at least 8 weeks, at least 16 weeks or at least 32 weeks in a lyophilised powder obtained by freeze drying an aqueous suspension of the particles. Any suitable method may conveniently be used to assess accessibility of the interior space. For example, a reporter mRNA or a labelled (for example, fluorescently tagged, immuno-labelled or radio-labelled) mRNA may be used to assess accessibility. According to certain embodiments, such stability may manifest in the ability of hybrid lipid particles of the invention to react with an active compound, especially an API (for example a mRNA) to produce liposomal particles of the invention. Liposomal particles of the invention preferably show good active compound (e.g., API) stability; for example, when kept at 4° C. for 3 months or 6 months, no more than 50% of the active compound, especially API, for example mRNA, will have degraded.

### Position of Inorganic Material

Hybrid lipid particles of the invention and liposomal lipid particles of the invention comprise particles of inorganic material (for example hydrolysable silicon particles). Preferably some of those particles are located exposed on the surface of the particles, some are located within the lipid bilayer, and some are located in the interior of the lipid particle. According to certain embodiments at least 10% of the total particles of inorganic material are located exposed on the surface of the particles, at least 10% of the total particles of inorganic material are located within the lipid bilayer and at least 10% of the total particles of inorganic material are located in the interior of the lipid particle.

### Arrangements Wherein the Particles of Inorganic Material are Aggregated

According to certain embodiments, the particles of inorganic material (for example particles of hydrolysable silicon) are present in hybrid lipid particles of the invention one or more aggregates. Such aggregates may optionally consist of between 10 and 200 particles. They may optionally comprise or consisting of chains of the particles, most especially chains that extend into the interior of the one or more lipid structures, such as into the interior of one or more of: micelles, incomplete micelles, liposomes, incomplete liposomes, and (e.g., solid or semi-solid) lipid globules (especially, liposomes and/or lipid globules).

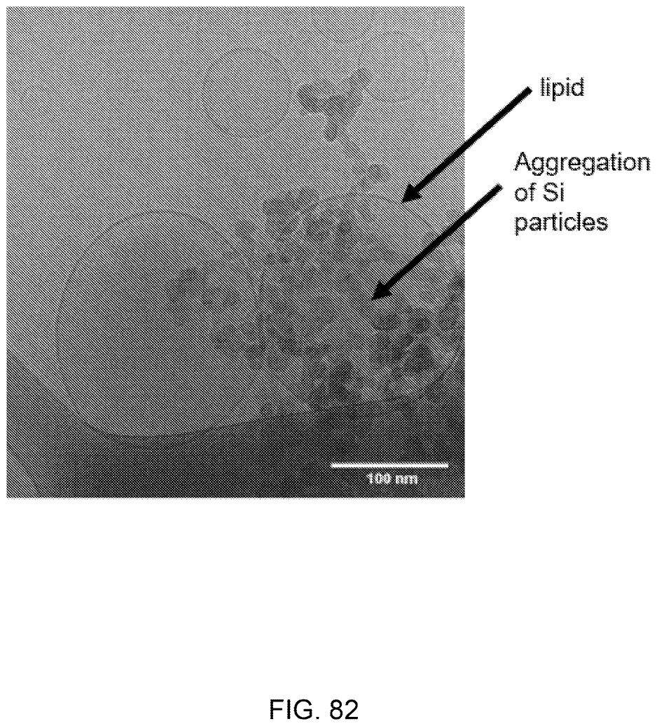

In embodiments using hydrolysable silicon, the particles of hydrolysable silicon may spontaneously coalesce into aggregates of particles, for example as shown in the transmission electron microscope (TEM) image of FIG. 82. Spontaneous aggregation is observed to be especially prominent when the particles of hydrolysable doped silicon have an average diameter of about 1 nm to about 50 nm, especially about 1 nm to about 30 nm, more especially about 5 nm to about 20 nm, such as about 10 nm. Such particle diameters may optionally be significantly below the typical diameter of hybrid lipid particles of the invention.

Thus, preferably, hybrid lipid particles of the invention may comprises aggregates (especially, chains) of the particles of hydrolysable doped silicon, wherein the particles comprising hydrolysable doped silicon have an average diameter of about 1 nm to about 50 num, especially about 1 nm to about 30 nm, more especially about 5 nm to about 20 nm, such as about 10 nm.

Aggregation may be occur particularly readily in methods of manufacture comprising extrusion such as those of the invention. Extrusion may optionally be or comprise extrusion through a porous membrane having average pore diameters of about 0.01 μm to about 1 μm, such as about 0.05 μm to about 0.6 μm. Preferably, extrusion occurs before addition of the API. Thus, the hybrid lipid particles may be formed in advance of the optional addition of an API.

As used herein, the term “aggregate of particles of hydrolysable silicon” may refer to a cluster of silicon particles wherein nearest-neighbour silicon particles are in contact with each other. Such clusters may have varying configurations, such as substantially spherical clusters of particles and/or chains of particles. Particularly preferred are configurations comprising or consisting of chains of particles.

Thus, hybrid lipid particles of the invention may comprise one or more aggregates of hydrolysable silicon particles. Preferably, the aggregates comprise one or more chains of the particles.

The one or more aggregates of hydrolysable silicon particles may be associated with the one or more lipids, for example embedded in lipid and/or attached to the surface of lipid.

The one or more aggregates of hydrolysable silicon particles may be associated with one or more lipid structures described herein, for example embedded within and/or attached to one or more lipid structures including micelles, incomplete micelles, liposomes, incomplete liposomes and lipid globules. Thus, the one or more aggregates of hydrolysable silicon particles may be associated with (for example, embedded in and/or attached to the surface of) one or more of lipid micelles, incomplete lipid micelles, liposomes, incomplete liposomes and lipid globules. In particular, the one or more aggregates of particles of hydrolysable silicon may be embedded in or attached to the surface of one or more of: liposomes; incomplete liposomes; and lipid globules.

The ratio of the longest dimension of an aggregate to the longest dimension of a lipid structure may on average be about 1:5 to 5:1, especially about 1:3 to 3:1; especially when the lipid structure is or comprises liposomes and/or lipid globules and the one or more aggregates are embedded therein or attached to the surface thereof. This may be measured, for example, by TEM as shown in FIG. 82.

The average longest dimension of an aggregate may be about 50 nm to about 500 nm, especially about 50 nm to about 200 num, such as about 50 to about 150 nm, such as when measured by TEM. In particular, the one or more aggregates may be or comprise one or more chains of particles, with the average length of a chain being about 50 nm to about 500 nm, especially about 50 nm to about 200 num, such as about 50 to about 150 num. The average cross-sectional diameter of a chain may be about 5 nm to about 50 nm, such as about 5 nm to about 30 nm.

The ratio of the longest dimension of an individual particle of hydrolysable silicon to the longest dimension of a lipid structure may on average be in a range of from about 1:100 to about 1:2, especially about 1:100 to about 1:5, more especially about 1:100 to about 1:9; especially when the lipid structure is or comprises liposomes and/or lipid globules and the one or more aggregates are embedded therein or attached to the surface thereof. This may be measured, for example, by TEM as shown in FIG. 82.

When the one or more aggregates are present, the average (e.g., mean) diameter of a particle within that aggregate may preferably be about 1 nm to about 50 nm, especially about 1 nm to about 30 nm, more especially about 5 nm to about 20 nm, such as about 10 nm. Additionally or alternatively, the particles may be porous and may have an average (e.g., mean) pore diameter of about 0.1 to about 5 nm, such as about 2 nm.

### Activation of Inorganic Material

According to certain preferred embodiment of all aspects of the invention the particles of inorganic material are particles of hydrolysable silicon material. Preferably such particles are activated by use of a solvent. “Activation” by a solvent or a solvent mixture comprises surface treatment of particles comprising hydrolysable silicon. Surface treatment with a solvent or solvent mixture is to be understood as comprising surface treatment with one or more alcohols. Especially preferred alcohols include methanol, benzyl alcohol and methanol (or mixture thereof). “Surface treatment” of hydrolysable silicon may comprise formation of Si—O(CH₂)ₓCH₃ (where x is zero for methanol, 1 for ethanol, etc), Si—(CH₂)ₓCH₃ and/or Si—H moieties on the particles' surface, where —(CH₂)ₓCH₃ may be replaced by other carbon-containing groups, such as an aromatic ring, for example when the alcohol is or comprises benzyl alcohol). According to certain embodiments of methods of the invention, the silicon nanoparticles are filtered immediately after activation in order to reduce their aggregation.

### Configuration of the API in the Liposomal Particles of the Invention

Liposomal particles of the invention comprise an active compound, especially an API. That API may be located within the interior space of the particle (i.e., encapsulated) and/or in non-covalent association with the exterior surface of the particle. According to certain embodiments at least 10% of the API is located within the interior space of the particle (i.e., encapsulated) and/or at least 10% of the API is located in non-covalent association with the exterior surface of the liposomal particle. According to certain embodiments at least 10%, 20%, 30%, 40% or 50% of the API is fully encapsulated within the interior space of the particle, with the remainder optionally being located in non-covalent association with the exterior surface of the liposomal particle.

Thus, hybrid lipid particles of the invention may comprise the particles of hydrolysable silicon associated with one or more liposomes and/or one or more incomplete liposomes, wherein the API is associated with (especially, bound to) the particles of hydrolysable silicon. An amino acid (especially, glycine, arginine and/or tyrosine, such as glycine) may also be associated with the particles of hydrolysable silicon.

The optionally present liposomes or incomplete liposomes, may have a mean diameter in a range of from about 50 nm to about 400 nm, especially about 50 nm to about 200 nm, more especially about 60 nm to about 100 nm.

API may be bound non-covalently to particles of hydrolysable silicon that are bound to the surface of one or more liposomes and/or one or more incomplete liposomes. Up to about 10 or 20% of the API may be bound non-covalently to particles of hydrolysable silicon that are, in turn, bound to the surface of one or more liposomes and/or one or more incomplete liposomes.

API may be bound non-covalently to particles of hydrolysable silicon that are, in turn, in the interior of one or more liposomes and/or one or more incomplete liposomes. At least about 50, 60 or 70% of the API may preferably be bound non-covalently to particles of hydrolysable silicon that are in the interior of one or more liposomes and/or one or more incomplete liposomes.

Preferably, API (especially, RNA, most especially mRNA) is bound non-covalently to particles of hydrolysable silicon that are in the interior of one or more liposomes and/or one or more incomplete liposomes; and API (especially, RNA, most especially mRNA) is bound non-covalently to particles of hydrolysable silicon that are bound to the surface of one or more liposomes and/or one or more incomplete liposomes.

In some embodiments, hybrid lipid particles of the invention may be free or substantially free of liposomes; and/or may be free or substantially free of incomplete liposomes.

Optionally, the one or more lipids may be formed of, or may comprise, one or more lipid monolayers. Optionally, the one or more lipids may be or comprise one or more micelles or incomplete micelles. It will be understood that micelles have similar characteristics to liposomes, except that micelles' walls are formed of lipid monolayer; whereas liposomes' walls are formed of lipid bilayer. Thus, a micelle may refer to a vesicle having at least one lipid monolayer, which may be approximately spherical in shape. A micelle, similarly to a liposome, may be visualised as a lipid “bubble” surrounding an interior space. The interior space may be a hydrophilic environment.

Thus, hybrid lipid particle so the invention may comprise the particles of hydrolysable silicon associated with one or more micelles and/or one or more incomplete micelles, wherein the API is associated with (especially, bound to) the particles of hydrolysable silicon.

The optionally present micelles may have a mean diameter in a range of from about 50 nm to about 400 nm, especially about 50 nm to about 200 nm, more especially about 60 nm to about 100 nm.

API may be bound non-covalently to particles of hydrolysable silicon that are bound to the surface of one or more micelles and/or one or more incomplete micelles. Up to about 10 or 20% of the API may be bound non-covalently to particles of hydrolysable silicon that are, in turn, bound to the surface of one or more micelles and/or one or more incomplete micelles.

API may be bound non-covalently to particles of hydrolysable silicon that are, in turn, in the interior of one or more micelles and/or one or more incomplete micelles. At least about 50, 60 or 70% of the API may preferably be bound non-covalently to particles of hydrolysable silicon that are in the interior of one or more micelles and/or one or more incomplete micelles.

Preferably, API (especially, RNA, most especially mRNA) is bound non-covalently to particles of hydrolysable silicon that are in the interior of one or more micelles and/or one or more incomplete micelles; and API (especially, RNA, most especially mRNA) is bound non-covalently to particles of hydrolysable silicon that are bound to the surface of one or more micelles and/or one or more incomplete micelles.

In some embodiments, hybrid lipid particles of the invention may be free or substantially free of micelles; and/or may be free or substantially free of incomplete micelles.

The one or more lipids may be formed of, or may comprise, one or more lipid globules, each globule optionally being surrounded by a layer of surfactants. Lipid globules do not enclose an interior space or cavity. Instead, they are solidly formed, or substantially solidly formed, of lipid, in which other components, such as the particles of hydrolysable silicon to which are bound API molecules, may be dispersed. Thus, the globules' interior may be studded with the particles of hydrolysable silicon; with API molecules, in turn, being bound (non-covalently) to the particles of hydrolysable silicon. Additionally or alternatively (preferably, additionally), the particles of hydrolysable silicon, to which are (non-covalently) bound API molecules, may be bound to the surface of one or more lipid globules.

Thus, hybrid lipid particles of the invention may comprise particles of hydrolysable silicon associated with (especially, dispersed within and/or bound onto the surface of) one or more (solid or substantially solid; i.e., non-hollow) lipid globules, wherein the API is associated with (especially, bound to) the particles of hydrolysable silicon.

The optionally present lipid globules may have a mean diameter in a range of from about 50 nm to about 400 nm, especially about 50 nm to about 200 nm, more especially about 60 nm to about 100 nm.

API may be bound non-covalently to particles of hydrolysable silicon that are bound to the surface of one or more lipid globules. Up to about 10 or 20% of the API may be bound non-covalently to particles comprising hydrolysable silicon that are, in turn, bound to the surface of one or more lipid globules.

API may be bound non-covalently to particles of hydrolysable silicon that are, in turn, in the interior of one or more lipid globules. At least about 50, 60 or 70% of the API may preferably be bound non-covalently to particles of hydrolysable silicon that are in the interior of one or more lipid globules.

Preferably, API (especially, RNA, most especially mRNA) is bound non-covalently to particles of hydrolysable silicon that are in the interior of one or more lipid globules; and API (especially, RNA, most especially mRNA) is bound non-covalently to particles of hydrolysable silicon that are bound to the surface of one or more lipid globules.

### Relative Dimensions

According to all aspects of the invention, the hybrid lipid particles or the liposomal lipid particles have a mean dimeter at least two times that of the mean diameter of the particles of inorganic material. In some embodiments, the relative sizes may differ more than this, for example the liposomal lipid particles may have a mean dimeter at least 3, 4, 5, 6, 7, 8, 10, 12, 15, or 20 times that of the mean diameter of the particles of inorganic material. In some embodiments, the liposomal lipid particles may have a mean dimeter at least 3 to 10 times that of the mean diameter of the particles of inorganic material or at least 5 to 20 times that of the mean diameter of the particles of inorganic material. In some embodiments, the hybrid lipid particles or the liposomal lipid particles have a mean diameter of between 50 nm and 400 nm, while the particles of inorganic material (for examples particles of hydrolysable silicon) have a mean diameter of between 10 nm and 60 nm (whilst the relative diameters remain within the limits recited).

### Note on Mean Diameters

This specification describes particle sizes as mean diameters. Particle diameters may be measured by any suitable method including Dynamic Light Scattering, electron microscopy and size exclusion methods. Preferably, the particles have a distribution of diameters around the mean diameter that is such that 80% of the particles have a diameter within 25%±the mean diameter. This is especially the case following filtration and extrusion which is known to increase mono-dispersity.

### Extrusion Membranes

Any suitable extrusion membranes may be used in accordance with the invention.

Preferably, the extrusion membranes are used as part of an in-flow extrusion system comprising an extrusion membrane and a flow pump (for example an HPLC pump). Preferred extrusion membrane include extrusion membranes comprising polycarbonate. Preferred pore sizes include those between 0.05 μm and 1.2 μm, for example between 0.08 μm and 1.0 μm, for example pore sizes of 0.8 μm, 0.4 μm or 0.1 μm.

### Extrusion Methods

Flow rate may depend on the total volume to be extruded and the area of extrusion membrane used. For example, a flow rate of 10 to 100 ml/min may be used. Extrusion pressure may be chosen to achieve sufficient flow rates. Extrusion is typically carried out at an elevated temperature. This is necessary in order to increase the fluidity of the lipids used. The precise optimal temperature may depend on the exact lipid formulation used, but the extrusion will typically be preferred to be carried out between 50° C. and 70° C., for example between 55° C. and 65° C., for example at about 60° C.

According to certain preferred embodiments of the invention, multiple extrusions are typically carried out. For example, at least 6, or at least 8 extrusions may be carried out on the same material. According to certain embodiments it is preferred that multiple extrusions be carried out using membranes of decreasing pore size. For example, multiple extrusions may be carried out with a membrane having a relatively large pore size, followed by multiple extrusions with a membrane having medium pore size, followed by multiple extrusions with membrane having relatively small pore size. In certain preferred embodiments, multiple extrusions (for example at least 2 extrusions, or at least 3 extrusions) are carried out using a membrane having a pore size between 0.6 μm and 1.0 μm (for example 0.8 μm), followed by multiple extrusions (for example at least 2 extrusions, or at least 3 extrusions) carried out using a membrane having a pore size between 0.3 μm and 0.5 μm, followed by multiple extrusions (for example at least 2 extrusions, or at least 3 extrusions) carried out using a membrane having a pore size between 0.08 μm and 0.2 μm (for example 0.1 μm).

### Reduction of Inorganic Material by Extrusion

As noted above, the invention is based, in part, on the discovery that particles of inorganic material (such as particles of silicon containing material) have a tendency to be removed from the hybrid lipid particles of the invention during extrusion but that this does not matter for the sustained stability and advantageous properties of the hybrid lipid particles. It appears that provided the particles of inorganic material are present at an initial relatively higher concentration, that concentration can be reduced without detriment during the extrusion. Accordingly, in certain embodiments of the first aspect of the invention, the preferred the first aspect of the invention, the extrusion step (step B) results in the retention of at least 50%, at least 60% or at least 70% or 80% (by weight) of the particles of inorganic material present in step A.

### Lipid Components

According to all aspects of the invention, it is preferred that the mixture of lipids used to form hybrid lipid particles of the invention or comprising liposomal lipid particles or hybrid lipid particles of the invention consists of one or more cationic lipid or ionisable lipid with one or more further lipid selected from neutral lipids and polar lipids, and optionally one or more additional lipid components.

The charge on a lipid (and therefore its categorisation as, for example, cationic, anionic or zwitterionic) is preferably assessed at pH 7.4, for example, it may be assessed in a physiologically compatible pH7.4 phosphate buffer solution.

According to all aspects of the invention the mixture of one or more cationic lipid or, ionisable lipid with one or more neutral lipid or polar lipids comprises at least one cationic lipid or ionisable lipid. Preferably the total cationic lipid or ionisable lipid (as a molar ratio) is between 20 and 70% of the total lipid, for example between 30 and 60%, or between 40 and 60%.

According to certain embodiments the cationic lipid or ionisable lipid is a cationic lipid. The cationic lipid may be selected from the group consisting of DOTAP (dioleoyl-3-trimethylammonium propane, 18:1 TAP); DODAC (dimethyldioctadecylammoniumchloride): SA (stearylamine, octadecylamine) and DOTMA (9-(trimethyl[2,3-(dioleyloxy)propyl]ammonium chloride) and mixtures of any thereof. Mixtures comprising DOTAP are especially preferred. According to certain embodiments at least half or all of the cationic lipid is DOTAP.

According to other embodiments the cationic lipid or ionisable lipid is an ionisable lipid. The ionisable lipid may be selected from the group consisting of [(4-hydroxybutyl)azanediyl]di(hexane-6,1-diyl) bis(2-hexyldecanoate, heptadecan-9-yl 8-{(2-hydroxyethyl)[6-oxo-6-(undecyloxy)hexyl]amino}octanoate, 7-[(2-hydroxyethyl)[8-(nonyloxy)-8-oxooctyl]amino]heptyl 2-octyldecanoate, (6Z,9Z,28Z,31Z)-heptatriaconta-6,9,28,31-tetraen-19-yl 4-(dimethylamino)butanoate, and DODMA, and mixtures of any thereof.

According to certain embodiments, the cationic lipid or ionisable lipid component may be a mixture of one or more cationic lipid (for example one or more of the cationic lipids listed above) and one or more ionisable lipid (for example one or more of the ionisable lipids listed above).

The lipid blend may optionally further include one or more neutral or polar lipid. The neutral phospholipid DOPE (dioleoyl phosphorylethanolamine), PC (phosphatidyl chlorine) and lecithin (a mixture predominated by PC) are all examples of non-cationic phospholipids which may be used as the neutral or polar lipid in accordance with the invention.

According to certain preferred embodiments the lipid blend is wholly or predominantly a cationic lipid and a phospholipid. For example, the lipid blend may consist of DOTAP and DOPE in approximately equal amounts.

The lipid blend may optionally and additionally further comprise as an additional lipid component a conjugated lipid such as a PEG-lylated lipid and/or a steroid/sterol component such as cholesterol. According to certain preferred embodiments, the lipid blend does not contain a material amount of either a conjugated lipid (for example a PEGylated lipid) nor a material amount of a steroid/sterol. In is preferred in all aspects that the particles of the invention do not include cholesterol. In other embodiments, small amount of cholesterol may be present, for example cholesterol may constitute less than 10% less than 8%, less than 5%, less than 2%, less than 1% or less than 0.5% of the total lipid present (by weight).

Lipids are generally understood to include fatty acids and fatty acid derivatives, glycerolipids, glycerophospholipids, sphingolipids, saccharolipids and polyketides. As used in the present application, the term “lipid” may encompass lipidated oligopeptide (a term used interchangeably herein with the term lipopeptide) wherein a short peptide sequence (such as a peptide sequence having 3 to 20 amino acid residues, such as 5 to 15 amino acid residues, especially 3, 4, or 5 amino acid residues, and most especially 5 amino acid residues) is conjugated to one or more fatty acid chains (especially a fatty acid chain having a 10 to 24 carbon chain length, preferably, a 12 to 18 carbon chain length; for example a 14, 15 or 16 carbon chain length; for instance, the peptide moiety may optionally be lipidated with a palmitoyl, cetyl or myristoyl moiety).

The one or more lipids may thus comprise one or more lipidated oligopeptides. Preferably, the one or more lipidated oligopeptides each comprise a fatty acid chain having in the range of about 12 to about 18 carbon atoms.

Preferably, the one or more lipidated oligopeptides each comprise 3 to 20 amino acid residues. Thus, the lipidated oligopeptide may be a lipdated tetrapeptide, lipidated pentapeptide or lipidated hexapeptide.

Preferably, the amino acid residues include at least one amino acid residue (for example, about 2 or about 3 amino acid residues) that is cationic at a pH of about 7.4 (physiological pH), such, for example, as lysine or arginine. For example, the lipidated oligopeptide may include one or more (for example, about 2) lysine resides.

An especial example of a lipidated oligopeptide (“lipopeptide”) is palmitoyl-pentapeptide-4 (CAS number 214047-00-4; abbreviated as PAL-KTTKS).

Thus, preferably, the one or more lipids may comprise or be one or more lipidated oligopeptides, particularly those having one or more amino acid residues that is or are positively charged at a pH of about 7.4 (i.e., about physiological pH) such, for example, as one or both of lysine and arginine.

The lipidated oligopeptide may especially be used in combination with one or more phospholipids, such as DOPE or DPPC. The alkyl chain of a lipidated oligopeptide molecule may be assimilated in a phospholipid bilayer, while the surface of the bilayer is decorated with the peptide moiety. Without wishing to be bound by theory, it is thought that a peptide moiety of the lipidated oligopeptide can enable the targeting of one or more specific tissues and/or cells. Meanwhile, where the peptide moiety bears a positive charge at a pH of about 7.4 (i.e., about physiological pH), it may stabilise negatively charged APIs (e.g. nucleic acids, especially mRNA or siRNA).

The one or more lipids may be or comprise one or more of: one or more cationic lipids (e.g. DOTAP); one or more phospholipids (e.g. DOPE); and one or more polyethylene glycol (PEG) lipids (e.g. DSPE-PEG₂₀₀₀).

The one or more lipids may be or comprise one or more structural lipids (e.g. a cholesterol-based lipid). However, the one or more lipids may optionally exclude structural lipid. Thus, the one or more lipids may exclude sterols; especially, they may exclude cholesterol. It has been found that the presently disclosed compositions need not rely on these types of lipid, which traditional API delivery systems typically rely on. Thus, the compositions disclosed herein have the potential to provide alternatives to API delivery systems reliant on these types of lipids, especially cholesterol. Where cholesterol is not available or where its use is otherwise not possible (e.g., due to its effect in the body) this may be advantageous.

The one or more lipids may optionally include one or more of: phosphatidylcholine (PC); hydrogenated PC; stearylamine (SA); dioleoylphosphatidylethanolamine (DOPE); cholesteryl 3β-N-(dimethylaminoethyl)carbamate hydrochloride (DC-chol); 1,2-dioleoyl-3-trimethylammonium-propane (DOTAP); PEGylated 1,2-Distearoyl-sn-glycero-3-phosphoethanolamine (DSPE), such as DSPE-PEG₂₀₀₀; and derivatives thereof.

In certain embodiments, the lipid is selected from the group consisting of phosphatidylethanolamine (PE), phosphatidylcholine (PC), stearylamine (SA), or any combination thereof.

The lipid or lipids component may, in some embodiments, be or comprise a cationic lipid. The term “cationic lipid” refers to molecules having a net positive charge at pH 7.4 (physiological pH), having a cationic head group attached via some spacer to a hydrophobic tail. Examples include DTDTMA (ditetradecyl trimethyl ammonium), DOTMA (2,3-dioleyloxypropyl-1-trimentyl ammonium), DHDTMA (dihexadecyl trimethyl ammonium); dioleoyl-3-trimethylammonium propane (DOTAP); and stearylamine (SA). The positive charge may typically be stabilised by a negative counterion.

Thus, the one or more lipids may optionally be or comprise DOTAP. DOTAP exists in an S and an R enantiomeric form, and may be present as the S-, R-form or as a racemate. Optionally, of the total DOTAP present by weight, the R and S forms may be in approximately equal amounts (i.e. no more than about 60% of the total DOTAP present by weight, of either form). In other embodiments at least about 80, 90, 95, 98, or 99% of total DOTAP is in the R-form. In other embodiments at least about 80, 90, 95, 98, or 99% of total DOTAP is in the S-form.

Nonetheless, as described herein, doping of the silicon may enable less cationic lipid, such as DOTAP, to be used, compared to conventional compositions for API delivery (such as lipid nanoparticles which comprise cationic lipid).

Thus, the one or more lipids may optionally exclude cationic lipid. As described herein, cationic lipid may not be necessary when doped silicon, especially p-doped silicon, is used.

Thus, the one or more lipids may be or comprise one or more of: one or more phospholipids (e.g. DOPE); and one or more polyethylene glycol (PEG) lipids (e.g. DSPE-PEG₂₀₀₀).

Overall, the particles of inorganic material disclosed herein can provide the potential to use less lipid (especially less cationic lipid, such as less DOTAP) in API delivery vehicles, compared to conventional API delivery vehicles which do not contain such particles (e.g. conventional liposomal nucleic acid delivery vehicles, such as those typically used for mRNA delivery in vivo). Additionally, or alternatively, the particles of inorganic material can provide the potential for API delivery vehicles to be formulated with a wider range of lipids while still providing transfection efficiency, storage stability, and/or targeted delivery to a particular type of tissue, or to a particular type of cell. In turn, this may lead to reduced reliance in the field on specific lipids, particularly cationic lipids, especially cationic lipids which are formulated specifically for the purpose of API delivery, and which may therefore not be cost-effective or easily accessible.

The one or more lipids may have an average molecular weight in the range of about 500 to about 1000.

The ratio of the one or more lipids (by which is meant all lipid components in the composition) to silicon may be in a range of from about 40:1 to about 1:1, especially a range of about 20:1 to about 1:1; such, for example, as a ratio of about 16:1, when the components are assembled for manufacture of a delivery system, i.e. before any further processing is carried out.

As described herein, the one or more lipids may especially comprise or be a phospholipid. The term “phospholipid”, as used herein, may refer to a lipid comprising a fatty acid chain and a phosphate group. Phospholipids may carry a negative charge, unlike a cationic lipid which is positively charged. However, phospholipids are typically zwitterionic compounds comprising both positive and negatively charged components, resulting in no overall charge. As such, phospholipids are typically classified as neutral lipids.

Suitable phospholipids may be or include glycerophospholipids. Especially suitable phospholipids may be or include those in which the polar head group is linked to quaternary ammonium moieties, such as phosphatidylcholine (PC) or hydrogenated phosphatidylcholine. The phospholipid may be, or be derived from, lecithin. A preferred phospholipid is DOPE (phosphatidyl ethanolamine or 1,2-dioleoyl-sn-glycero-3-phosphoethanolamine).

Preferably, the side chain(s) of the phospholipid may be aliphatic side chain(s) with about 15 or more carbon atoms, or an ether side chain with about 6 or more repeating ether units, such as a polyethylene glycol or polypropylene glycol chain.

Lipids with either side chains may be referred to as “PEG-lipids” or “PEG-ylated” lipids. Thus, as used in the present application, the term “lipid” may cover PEG lipids. Thus, according to preferred embodiments, the one or more lipids may comprise or be one or more polyethylene glycol (PEG) lipids, especially PEGylated DSPE, such as DSPE-PEG₂₀₀₀.

The one or more lipids may optionally comprise or consist substantially of phosphatidylcholine (PC), hydrogenated phosphatidylcholine, stearylamine (SA), or combinations thereof.

The one or more lipids may optionally comprise at least about 5% (e.g., at least about 30% or at least about 50%) by weight of PC, based on the total weight of the one or more lipids.

The one or more lipids may optionally comprise at least about 5% (e.g., at least about 30% or at least about 50%) by weight of hydrogenated PC, based on the total weight of the one or more lipids.

The one or more lipids may optionally comprise at least about 5% (e.g., at least about 30% or at least about 50%) by weight of SA, based on the total weight of the one or more lipids.

The one or more lipids may optionally comprise or consist essentially of PC and SA, optionally in a ratio by weight of PC to SA in a range of from about 1:1 to about 20:1.

The one or more lipids may optionally comprise or consist essentially of the combination of DOPE, SA, and DC—

In certain preferred embodiments, the one or more lipids may optionally comprise or consist essentially of a combination of DOTAP, DOPE and a PEG-lipid (especially DSPE-PEG₂₀₀₀).

The ratio by weight of DOTAP:DOPE may be in a range of from about 1:2 to about 2:1; such, for example, as about 1:1. The ratio by weight of DOTAP: PEG-lipid may be in a range of from about 10:1 to about 5:1; such, for example, as about 7:1. The ratio by weight of DOPE:PEG-lipid may be in a range of from about 10:1 to about 5:1; such, for example, as about 7:1.

### Additional Components

It has been found that the stability of the hybrid lipid particle and of the liposomal lipid particle of the invention can be increased further in the presence of one or more amino acids and/or one or more non-reducing disaccharides; thus, the stability of the active compound (especially, an API) in the liposomal lipid particle of the invention can be increased further. Therefore, methods of the invention may optionally be carried out in the presence of one or more amino acid and/or one or more non-reducing disaccharide. The lipid particles of all aspects of the invention may further comprise a non-reducing disaccharide and/or an amino acid. A preferred non-reducing disaccharide is trehalose. A preferred amino acid is glycine. In certain preferred embodiments the use of both glycine and trehalose is preferred. These components may be able to electrostatically coordinate with inorganic material such as silicon and increase the overall stability of the system. Glycine is especially preferable due to its hydrophilic, medium to moderately nonpolar nature and because in aqueous solutions at physiological pH (circa pH 7.4), glycine exists as a zwitterion. Glycine binds to the surface of inorganic material (for example silicon) and is able, because of its zwitterionic nature, to electrostatically coordinate, with both positive and negative charges and partial charges, which enhances the particle stability.

In its broadest sense, the term “amino acid” encompasses any artificial or naturally occurring organic compound containing an amine (—NH2) and carboxyl (—COOH) functional group. It includes α, β, γ and δ amino acids. It includes an amino acid in any chiral configuration. The amino acid may, especially, be a naturally occurring a amino acid. It may be a proteinogenic amino acid or a non-proteinogenic amino acid (such as carnitine, levothyroxine, hydroxyproline, ornithine or citrulline).

The one or more amino acids may help stabilise the particles of inorganic material themselves, especially silicon. In vivo, the one or more amino acids may help to modulate the rate of hydrolysis of a silicon particle, such that the silicon hydrolyses to bioavailable ortho silicic acid (OSA) degradation product; rather than insoluble polymeric hydrolysis products. Controlling the rate of hydrolysis of silicon in vivo may influence the rate of release of an active compound (especially, an API).

Controlling the rate of active compound (especially, API) release may modulate the length of the time period during which protection of the active compound (especially, API) is sustained, especially concerning protection in vivo in the presence of various bodily fluids. Thus, more of the active compound (especially, API) may be delivered to a target cell in a given time period, than for an otherwise identical composition.

In preferred embodiments, the amino acid(s) may comprise or consist essentially of glycine.

Additionally, or alternatively, amino acids which are neutral or positively charged at physiological pH (about pH 7.4), such as tyrosine or arginine, may help to stabilise negatively charged APIs (e.g. nucleic acids such as mRNA). Meanwhile, amino acids which are neutral or negatively charged at physiological pH (about pH 7.4) may help to stabilise positively charged APIs. Nonetheless, the interplay of charge-based and/or other interactions (such, for example, as sterics) resulting from the combination of inorganic material, lipid(s) and amino acid(s) may be such that amino acid(s) which are positively charged at physiological pH may help to stabilise positively charged active compounds (especially, APIs), or amino acid(s) which are negatively charged at physiological pH may help to stabilise negatively charged active compounds, especially negatively charged APIs. The ratio by weight of the one or more lipids (i.e. total lipid components) to the amino acid(s) may be in a range of from about 40:1 to about 1:1; such, for example, as about 32:1.

Optionally, the composition may specifically comprise the amino acid tyrosine in addition to the amino acid(s) described hereinabove. Optionally, the composition may specifically comprise tyrosine instead of the amino acid(s) described hereinabove. Thus, it will be understood that while tyrosine is an amino acid, it may optionally be present as a separate, further component, for the purposes of the present disclosure, distinct from the amino acid(s) described hereinabove. Thus, when tyrosine is present as a further component distinct from and in addition to the amino acid(s) described hereinabove it will be understood that the calculation of a range of the one or more lipids to amino acid(s) of from about 40:1 to about 1:1; such, for example, as about 32:1, disclosed above, does not include the amount of additional, distinct tyrosine.

Additionally, or alternatively, there may be included one or more non-reducing disaccharides, especially trehalose. The ratio by weight of the one or more lipids (i.e. total lipid components) to non-reducing disaccharide may be in a range of from about 20:1 to about 1:1; such, for example, as about 16:1.

### Stability of Particles

The hybrid lipid particles of the invention and the liposomal particles of the invention show enhanced dimensional stability compared to corresponding particles without the particles of inorganic material (for example hydrolysable silicon material) according to the invention. This enhanced dimensional stability manifests in a resistance of the particles to coalescence into larger particles. According to certain embodiments, the rate of coalescence at 5° C. is at most half that of equivalent corresponding particles (of identical composition but for the absence of particles of inorganic material (for example, hydrolysable silicon particles according to the invention). According to certain embodiments, at least 90% of particles have not coalesced and have maintained their original size after 3 months' storage in aqueous solution at physiological pH (of about 7.4) at 5° C.

### Stability of Charge

The surface charge of lipid particles, including hybrid lipid particles and liposomal lipid particles of the invention, may be estimated using the parameter of zeta potential (electrokinetic potential). As a rule of thumb, a suspension of particles in nuclease free water having a low zeta potential (0 to +5 mV) is unstable and rapidly coalesces. Values of ±30 mV to ±40 mV correspond to reasonable stability and values ±40 mV to ±60 mV good stability, values above ±60 mV correspond to excellent stability.

According to certain embodiments, the hybrid lipid particles of the invention and the liposomal lipid particles of the invention have values of >±40 mV, more preferably >±45 mV, >±50 mV, or >±60 mV. Preferably the zeta potential is increased by at least ±10 mV by the presence of hydrolysable silicon (that is to say the zeta potential is at least ±10 mV greater than ±10 mV than that of equivalent lipid particles which are identical to those of the invention but for the absence of particles of inorganic material (such as hydrolysable silicon particles according to the invention).

The presence of the inorganic material such as hydrolysable silicon also inhibits loss of positive charge of the cationic lipids. This is known as lipid ageing and preferably is slowed (at 5° C.) by at least a factor of 2, 4, 8 or 16 in lipid particles of the invention.

### Stability of Nucleic Acid

The liposomal lipid particles of the invention act to protect active compounds and APIs. The liposomal lipid particles of the invention especially act to protect active compounds and APIs which are nucleic acids (in particular RNA, more especially mRNA) which is complexed electrostatically inside and on the surface of the liposomal lipid particles. The invention allows therapeutic formulations such as vaccines to be stored with greater ease; for example, to be stored at 5° C. or room temperature as opposed to sub-zero temperatures. They also increase stability and reduce nucleic acid degradation during freeze-drying, rehydration, transport and storage. According to certain embodiments of the invention, the half-life of an active compound or API, especially wherein that active compound or API is an mRNA is extended by a factor of at least 100, at least 1000 or at least 10000 compared with a corresponding mRNA not complexed with a liposomal lipid particle of the invention. According to certain embodiments, the half-life of an mRNA is extended by a factor of at least 10, at least 100 or at least 1000 compared to a corresponding mRNA complexed with an equivalent liposomal lipid particle lacking the particles of inorganic material (for example, hydrolysable silicon) component according to the invention. Half-lives may be measured at pH 7.4 at 5° C. in aqueous physiologically compatible solution. According to certain embodiments, especially wherein the active compound or API is a nucleic acid such as an RNA, the half-life may be measured in a natural physiological lipid. For example, it may be measured in vivo or ex vivo or in vitro in blood or in a blood component such as plasma. For example, it may be measured in vitro in a human blood plasma. Such an assay may be especially demanding for an active ingredient or API which is a RNA, because it is known that human blood plasma contains substances, for example enzymes, known to degrade RNA.

### Methods of the Invention

Methods of manufacturing an aqueous suspension of hybrid lipid particles comprise Step A, the mixing of one or more lipids in a solvent or solvent mixture and a suspension of particles of inorganic material in a solvent or solvent mixture into an aqueous medium. This step may optionally be carried out using one or more different devices and methods, including bulk mixing methods or devices and microfluidic mixing methods or devices.

### Optional Steps

Methods of the invention may optionally include—subsequent to Step B and, if present, Step C—an additional Step D of contacting the hybrid lipid particles of the invention with an active compound, especially a pharmaceutically active ingredient (API). Such a method optionally results in the production of liposomal lipid particles of the invention. According to certain embodiments of methods of the invention, there is an interval of at least 1 week, at least 1 month or at least 6 months between Step B and, if present, Step C, and step D. Optionally, this the hybrid lipid particles are maintained at 4° C. or at 20° C. for that interval.

### Optional Filtration

Methods of the invention may optionally include—subsequent to Step B—an additional Step C of purifying, concentrating and/or sterilizing the suspension by tangential flow filtration.

Thus, according to the first aspect of the invention an additional Step C may be interposed between steps B and D. Preferably Step C is carried out using tangential flow filtration although any suitable filtration process could be used. One purpose of filtration is to increase the size uniformity of the hybrid lipid particles by means of filtration through a membrane having a size cut-off at the desired particle size (for example 100 nm). Surprisingly, it has also been found that filtration removes from the solution many of those particles of inorganic material (for example particles of hydrolysable silicon) which are not strongly associated with the hybrid lipid particles, i.e. those which are not bound on the surface of or in the lipid particles. This is despite the particles of inorganic material (for example hydrolysable silicon particles) being smaller than the hybrid lipid particles and the filtration membrane exclusion size. Accordingly, an optional filtration step may be carried out between steps B and C of the invention in its first aspect.

After filtration, the total proportion of the weight of the hybrid lipid particle made up from the particles of inorganic material (for example hydrolysable silicon particles) may be less than 20%, for example less than 10% or less than 5% or 1%. By keeping the amount of inorganic material such as silicon low, concerns about side effects of the inorganic material such as silicon are mitigated.

According to certain embodiments the optional filtration is diafiltration. In certain embodiments this diafiltration may use a diafiltration solution containing both a non-reducing disaccharide such as trehalose and an amino acid such as glycine is used to maintain the concentration of these materials in the product.

### Absence of Solvent Evaporation Steps

Methods of the invention do not necessarily require a step of solvent evaporation. According to preferred embodiments of method so the invention, such methods do not contain a step of solvent evaporation. Specially, such methods do not contain a step of evaporation of solvent used to activate the particles of inorganic material (for example the particles of hydrolysable silicon) nor a step of evaporation of the solvent used to mix the one or more lipids. Preferably, substantially all solvent (especially all alcohol such as methanol) is removed by a filtration method as described herein without any material evaporation.

### In-Process Control Steps

According to preferred embodiments of methods of the invention, such methods additionally comprise one or more optional in-process quality control steps. Methods may optionally comprise a visual check for complete dissolution of lipids before mixing in step A. Alternatively or additionally, methods may optionally comprise subsequent to step B, measurement of dynamic a light scattering (DLS) parameters an average hydrodynamic size parameter, a measurement of polydispersity (PDI) and/or a measurement of zeta potential Optionally, this measurement is them compared to a product specification and of it fails to meet the product specification, extrusion step B is optionally repeated, followed by an optional repeat of the measurement and a repeat of the comparison to product specification.

### Pharmaceutical Compositions and Their Uses

The invention further contemplates the use of hybrid lipid particles of the invention, and liposomal lipid particles of the invention to formulate pharmaceutical products which also fall within the scope of the invention. Such pharmaceutical products include injectable formulations (such as injectable vaccines), topical creams, capsules, tablets and ointments. They also include pharmaceutical precursors or products for example dehydrated (lyophilized) and concentrated products which must be diluted and/or rehydrated prior to use.

### Temperatures

The temperature to be adopted during extrusion is generally about 60° C., for example between 50° C. and 70° C. Such temperatures threaten the integrity of certain APIs, including when the API is a nucleic acid such as RNA (siRNA, saRNA, mRNA) as is preferred in certain embodiments. As an example of a method of the invention there is first the step of producing a hybrid lipid particle of the invention in the absence of an API. There is subsequently the optional step of transporting the hybrid lipid particle for further use, of lyophilising it, freezing it or storing in before further use, followed by the step of contacting the hybrid lipid particle with an API to product a suspension of liposomal lipid particles of the invention. This final step is preferably carried out at a relatively low, and therefore more benign, temperature. For example, it may be carried out at room temperature (25° C.) or slightly above freezing (0° C.). According to certain embodiments it is carried out at between 0° C. and 30° C., for example between 0° C. and 25° C., or between 0° C. and 10° C.

### Other Method Features

According to certain preferred embodiments, the step A of methods of the invention wherein one or more lipids and particles of inorganic material are mixed into an aqueous medium, comprises mixing the one or more lipids into the aqueous medium wherein the one or more lipids are provided in a solvent (for example an alcohol such as methanol). The use of such a solvent advantageously ensures adequate mixing between the one or more lipids. It might be thought that the solvent would need to be allowed to evaporate, especially if it was a toxic solvent such as methanol. It might be thought that the presence of solvent would be detrimental to the formation of the lipid particles. The present inventors have found surprisingly that good formation of hybrid lipid particles takes place even when the solvent is not evaporated and the one or more lipid and particles are mixed into an aqueous medium according to step A of a method of the invention, wherein the one or more lipids are provided in a solvent for example an alcohol such as methanol. Such methods preferably comprise a downstream step of purifying the suspension by tangential flow filtration (for example a method as set out in step C of a method of the invention). It has been found that such a step is suitable for removing the solvent from the suspension. If an activating solvent or solvent mixture is used to activate the particles of inorganic material (such a solvent may be the same solvent or the same solvent mixture as the solvent or solvent mixture in which the lipid are provided) TFF may be used in a subsequent step to remove activating solvent. TFF may optionally be used in a diafiltration method. In certain embodiments this diafiltration method may use a diafiltration solution containing both a non-reducing disaccharide such as trehalose and an amino acid such as glycine is used to maintain the concentration of these materials in the product.

### Mixing Methods

Lipid particles may be manufactured by various processes, Prior art methods often use methods which include the hydration of lipid thin films. Whilst such methods can be effective, they can be vulnerable to producing lipid particles with inconsistent sizing and low encapsulation efficiency. The present invention, for example in accordance with methods of the first aspect of the invention, mixes one or more lipids in a solvent or solvent mixture and a suspension of particles of inorganic material in a solvent or solvent mixture and then mixes the solvent or solvent mixture(s) carrying the one or more lipids an carrying the suspension of particles of inorganic material into an aqueous medium.

According to certain embodiments, this final mixing may be carried out by injecting the solvent or solvent mixture(s) into the aqueous medium or any other methods including bulk or microfluidics devices methods that offer effective and efficient mixing of lipids and inorganic particles of inorganic material.

According to certain preferred embodiments, it has been found that further improvements in production efficiency and product quality can be achieved by mixing the solvent or solvent mixture(s) and the aqueous medium in a rapid mixing method.

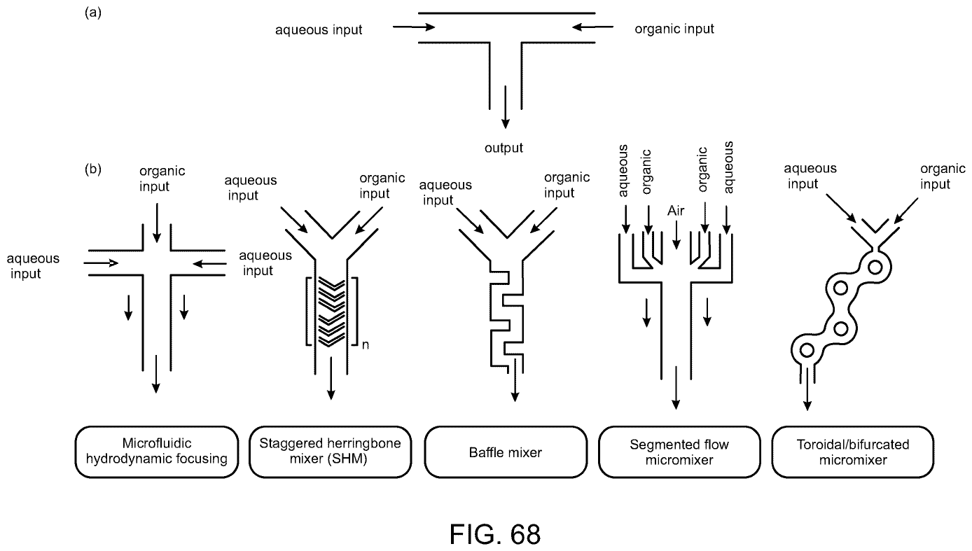

Rapid mixing methods according to embodiments of the invention include cross-flow injection or T-junction mixing. Such methos are an adaptation of the solvent-injection approach and result in rapid mixing of the organic can aqueous solutions/suspension. It has been found that they enable reliable production of small hybrid lipid particles, and that the particle size is conveniently controllable by varying the injection flow rate and/or pressure. FIG. 68 shows examples of various rapid mixing methods according to certain embodiments of the invention, especially in its first aspect. They include cross-flow injection, T-junction mixing, microfluidic hydrodynamic focusing, staggered herringbone mixing (SHM), baffle mixing, segmented flow micromixing and toroidal/bifurcated micromixing.

### Active Pharmaceutical Ingredients and Other Active Compounds

The active pharmaceutical ingredient of the invention in certain embodiments may be any pharmaceutically active compound. It is preferably a hydrophilic compound, for example a negatively charged compound, for example a nucleic acid. In other embodiments methods and products of the invention comprise other types of “active compound”. Such other active compounds may not necessarily be pharmaceutically active compounds. For example, an active compound may be a cosmetically useful compound, a research tool, or a plant protection compound. It is preferably a hydrophilic compound, for example a negatively charged compound, for example a nucleic acid.

An API or other active compound according to the invention may, for example, be a fragile compound. As used herein, the terms “reactive compound” and “fragile compound” may be interchangeable and may both refer to a compound which (i) is liable, upon storage for more than about a week at about 25° C., to degrade fully; and/or (ii) has a half-life in vivo of under about an hour.

An API may be any pharmaceutically active compound; thus, for example, it will be understood that the term “API” encompasses pro-drugs. Especially, the API may be a nucleic acid, more especially siRNA or mRNA. Meanwhile, in other preferred embodiments, the API may be a protein.

### Nucleic Acid for Use in the Invention

The invention is especially suitable for use with any nucleic acid, more especially RNA, because RNA, in the absence of the protection provided by the invention, is especially prone to degradation. Therefore, according to certain preferred embodiments of all aspects of the invention, the nucleic acid is RNA. RNA may optionally be siRNA. It may optionally be mRNA. For example, it may be mRNA encoding a vaccine antigen. RNA may optionally be chemically modified or sequence-modified to increase its stability and prevent its degradation. According to certain embodiments of the invention the RNA is chemically modified to increase its stability or to prevent its degradation. However, in certain preferred embodiments the RNA is not chemically modified because such treatment has been found to be unnecessary because the liposomal lipid particles of the invention may provide sufficient protection against RNA degradation such as RNA modification is not necessary.

According to certain embodiments of all aspects of the invention, the nucleic acid is DNA. According to other preferred embodiments, the nucleic acid is RNA. It may be siRNA or mRNA or saRNA or shRNA. It may be of any suitable length but typically may be between 10 and 30 nucleotides long for siRNA, saRNA or shRNA or 200 to 2000 nucleotides long for mRNA. It may be double or single stranded or, especially in the case of siRNA, saRNA or shRNA it may be chemically single stranded but with one or more regions of base pairing (and with optional unpaired overhangs). It may optionally be modified chemically (for example by use of N1-methylpseudouridine substitution) or have its sequence modified (for example by UTR-shortening). Preferably, the nucleic acid (i.e. RNA) may be unmodified (especially not chemically modified) as this may be unnecessary to provide stability. According to certain embodiments the nucleic acid may be RNA which may optionally comprise a 5-prime cap and/or a poly-A tail, but in which other modifications are absent.

An RNA according to the invention may be small interfering RNA (siRNA), small activating RNA (saRNA), small hairpin RNA (shRNA), or messenger RNA (mRNA), especially mRNA (e.g., mRNA that encodes a protein of a pathogenic organism).

Other nucleic acids for use in accordance with the present disclosure include: double- and single-stranded DNA; DNA:RNA hybrids; peptide:DNA hybrids; and peptide:RNA hybrids.

RNA and DNA may be naturally occurring or chemically modified to enhance their therapeutic properties, such as enhanced activity, increased serum stability, reduced off-targeting and lower immunological activation. Chemical modifications to RNA and DNA may include any modifications commonly known in the art.

Thus, as used herein, the terms nucleic acid, DNA and RNA also include known types of modifications, for example, labels which are known in the art, methylation, “caps”, substitution of one or more of the naturally occurring nucleotides with an analogue, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

Similarly, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine, e.g., isoguanine, isocysteine, and the like. In some embodiments, the oligonucleotides and/or probes include at least one, two, three or four modified nucleotides.

In some embodiments, the nucleic acids such as the RNAs disclosed herein include one or more universal bases. As used herein, the term “universal base” refers to a nucleotide analogue that can hybridize to more than one nucleotide selected from A, U/T, C, and G. In some embodiments, the universal base can be selected from the group consisting of deoxyinosine, 3-ntiropyrrole, 4-nitroindole, 6-nitroindole, 5-nitroindole.

In its broadest sense, the term “saRNA” encompasses small activating RNA, comprising RNA molecules which operate within the RNA activation (RNAa) pathway. The saRNA may be double-stranded. The saRNA may have a length in a range of about 5 to about 50 base pairs, especially about 10 to about 40 base pairs, more especially about 10 to about 30 base pairs.

In its broadest sense, the term “shRNA” encompasses small hairpin RNA, comprising RNA molecules which operate within the RNA interference (RNAi) pathway. The shRNA may be single stranded while also having base pairing thereby forming a hairpin loop. The single strand of the shRNA may have a length in a range of about 10 to about 100 bases, especially about 25 to about 75 base pairs, more especially about 40 to about 70 base pairs; which may then form a hairpin loop.

In its broadest sense, the term “siRNA” encompasses small interfering RNA, comprising RNA molecules which operate within the RNA interference (RNAi) pathway. siRNA is sometimes known as short interfering RNA or silencing RNA. The siRNA may be double stranded. The siRNA may have a length in a range of from about 5 to about 50 base pairs, especially about 10 to about 40 base pairs, more especially about 15 to about 30 base pairs. In its broadest sense, the term “mRNA” encompasses messenger RNA for the synthesis of protein(s). It may encompass mRNA comprising a 5-prime cap and/or a poly-adenylated terminus. Alternatively, one or both of those features may be absent. Typically, the mRNA may be single stranded. The coding region of the mRNA may be at least about 100, especially at least about 500, more especially at least about 1000 bases in length.

The mRNA may encode an antigen, thereby providing a composition which is a vaccine. The antigen may be a viral antigen, especially a viral antigen of one of the viral diseases described hereinbelow; more especially an antigen of a respiratory virus, for example an antigen of SARS-CoV-2, for example an antigen deriving from the spike protein of SARS-CoV-2.

The mRNA may encode multiple proteins, thereby providing more effective pharmacological activity. The mRNA may encode multiple antigens, especially multiple viral antigens.

The mRNA may additionally encode an adjuvanting protein. An adjuvant may additionally or alternatively be provided as a further component of the composition in addition to the API.

### Complexation of Components, Especially Particle/Lipid/API Complexation

Preferably, the particles of inorganic material are complexed with the one or more lipids thus forming a delivery vehicle for transport of active compound or API. Thus, when the active compound or API is added, it also becomes complexed with the particles and/or the lipid. Put another way, the particles and lipid are organised into a delivery vehicle that is loaded with the active compound or API. Advantageously, this may make the active compound or API less liable to react with one or more external reactive species. The active compound or API may be less at risk of degradation catalysed by enzymes external to the complex, especially in vivo, such as during circulation in the body and/or in a cell cytoplasm; this may especially be the case where the active compound or API is a nucleic acid, more especially mRNA.

In its broadest sense, as used here, the term “complexed with” may encompass ionic and/or covalent and/or physical interactions, and especially may encompass charge-charge interactions, such as those resulting from the particles' zeta potential.

Thus, preferably, the zeta potential of the particles, especially when modulated by the one or more lipids and any other components present, is such as to attract and facilitate binding of the active compound or API.

In preferred embodiments wherein amino acid(s) is or are present, the amino acid(s) may also complex with the particles, lipid(s) and/or active compound/API. The amino acid(s), especially when charged, may modulate the particles' zeta potential thus modulating active compound/API and/or lipid complexation with the particles.

### Tangential Flow Filtration

The methods of the invention optionally include a step of one or more tangential flow filtrations (TFFs). TFF may be configured to concentrate an aqueous suspension according to the invention. Additionally, or alternatively TFF may be used to remove a solvent, for example methanol, used in earlier stages of the method.

The use of TFF is especially preferred as part of certain preferred embodiments of methods the invention wherein those methods lack a step of solvent evaporation.

### Advantageous Properties and Product Parameters

Particles of the invention, both hybrid lipid particles and liposomal lipid particles preferably are sized between 60 and 120 nm with a polydispersity index (PDI) of between 0.100 and 0.200 and a zeta potential of between 50 and 70 mV.

### Configuration of Hybrid Lipid Particles and Liposomal Lipid Particles

Hybrid lipid particles of the invention and liposomal lipid particles of the invention, in all its aspects, preferably have the following configuration in addition to the lipid structure. The lipid particles may have particles of inorganic material (for example, hydrolysable silicon particles) both integrated in the lipid bilayer and also partially or wholly exposed on the hybrid or liposomal particle surface so as to be available to interact with active compound, especially API, more especially nucleic acid. Preferably, at least 10%, at least 20%, at least 30% or at least 50% of total particles of inorganic material (for example, hydrolysable silicon particles) are accessible at the surface of the lipid particle and are not fully encapsulated within the lipid structure. When present the active compound, especially nucleic acid or other API, is predominantly located electrostatically bound to the surface of the lipid particle. For example, over 90% of the total active compound (especially nucleic acid or other API) according to certain embodiments present will be bound to the surface of the lipid particles and less than 10% will be encapsulated within the lipid structure. In some embodiments zero or virtually zero (for example less than 0.5%) of the total active compound (especially nucleic acid or other API) present will be encapsulated within the lipid structure. In other embodiments there will be a more equal split between active compound or API which is encapsulated and that which is associated with the surface of the lipid particle. For example, according to some embodiments, at least 10% of the active compound or API will be associated with the surface of the particle and at least 10% of the active compound or API will be encapsulated.

### Methods of, and Products Relating to, Treatment

Products of the invention maybe used in methods of treatment or may be products for use in methods of treatment. Methods of the invention may further comprise subsequent steps constituting methods of treatment.

Methods of treatment include treatment or prevention of a disease or disorder. In some embodiments, methods of treatment may comprise down-regulation of gene expression by siRNA. In other embodiments, methods of treatment may comprise vaccination, for example, vaccination against a cancer or vaccination against an infectious disease by delivery of an mRNA encoding an antigen (or fragment thereof) or the causative agent of the infectious disease (for example the spike protein of SARS-CoV-2).

The disease or disorder may be an infectious disease. As used herein, the term “infectious” may be used to refer to a disease which is liable to be transmitted from one organism to another, especially from one human to another.

The infectious disease may be a viral, bacterial, fungal, or parasitic disease, especially a viral disease.

Where the disease is a viral disease, it may be that of a respiratory virus, such, for example, as respiratory syncytial virus (RSV), parainfluenza virus (HPIV), metapneumovirus (HMPV), rhinovirus (HRV), coronavirus such as SARS-CoV (especially SARS-CoV-1, more especially SARS-CoV-2), adenovirus (HAdV), enterovirus (EV), bocavirus (HBoV), parechovirus (HPeV) or an influenza virus.

The viral disease may be that of a dengue virus, Ebola virus, encephalomyocarditis virus, hepatitis virus, herpes virus, human immunodeficiency virus, human papillomavirus, human t-lymphotropic virus, measles virus, monkeypox virus, mumps virus, polio virus, rabies virus, rotavirus, rubella virus, varicella-zoster virus, west Nile virus, yellow fever virus or zika virus.

The disease or disorder may be a genetic disease or disorder.

In some embodiments, the genetic disorder may be characterised by a deficiency in the expression of one or more proteins, especially one or more enzymes.

The genetic disorder may be a multifactorial disorder, i.e. not confined to any specific pattern of single gene inheritance and likely to be associated with multiple genes effects together with the effects of environmental factors; such, for example, as schizophrenia, diabetes, asthma, depression, epilepsy, heart disease or hypothyroidism.

The genetic disorder may involve one or more mutations in one or more genes.

Thus, the genetic disorder may be a monogenic disorder, liable to occur if at least one mutation occurs in a single gene. Where the genetic disorder is a monogenic disorder, it may involve one mutation in the single gene or more than one mutation in the single gene. Examples of monogenic disorders include sickle cell anaemia, cystic fibrosis, Huntington's disease or Duchene muscular dystrophy.

The genetic disorder may involve one or more mutations in more than one gene. By way of non-limiting example, the genetic disorder may involve more than one mutation in a first gene and one mutation in a second gene.

The genetic disorder may be a disorder liable to occur if at least one mutation occurs in at least one gene amongst a set of genes; especially, such a genetic disorder may be osteopetrosis.

The genetic disorder may be Angelman syndrome; Canavan disease; Charcot-Marie-Tooth disease; colour blindness; cri du chat syndrome; cystic fibrosis; DiGeorge syndrome; Down syndrome; Duchenne muscular dystrophy; familial hypercholesterolemia; haemochromatosis type 1; haemophilia; Klinefelter syndrome; neurofibromatosis; phenylketonuria; polycystic kidney disease; Prader-Willi syndrome; Scheuermann's disease; sickle cell disease; spinal muscular atrophy; Tay-Sachs disease; or Turner syndrome.

In its broadest sense, as used herein, the term genetic disorder may encompass cancer. The cancer may be or involve a blood cancer (such, for example, as a leukaemia, a lymphoma, or a myeloma) or a solid tumour (such, for example, as a sarcoma; a carcinoma; a carcinosarcoma; or a lymphoma).

Thus, especially, the cancer may be a cancer of the blood, skin, brain, prostate, breast, lung, oesophagus, stomach, small intestine, pancreas, colon and/or rectum, central nervous system, urinary bladder, thyroid, kidney, uterine corpus, oral cavity, or ovary.

In more detail, the cancer may be or involve a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an oesophageal cancer, a hepatocellular cancer, a gastric cancer, a gastrointestinal stromal tumour (GIST), a colon cancer, a colorectal cancer, an ovarian cancer, a melanoma, a neuroectodermal tumour, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumour, diffuse type giant cell tumour, or glioblastoma.

### Preparation, Storage, Stability and Administration of the Disclosed Pharmaceutical Compositions (Also Referred to Simply as “Compositions”)

Treating or preventing a disease or disorder according to the invention may comprise administering a prophylactically effective amount of a pharmaceutical composition disclosed herein to the subject (especially a human subject), wherein the subject is in need thereof; for example, identified by a physician or other healthcare practitioner as being in need thereof. Meanwhile, treating a disease or disorder in a human subject may comprise administering a therapeutically effective amount of a pharmaceutical composition disclosed herein to a subject in need thereof.

Dosage amounts of a pharmaceutical composition disclosed herein may be varied so as to obtain an amount of an API which is effective to achieve the desired prophylactic and/or therapeutic response for a given subject, without being toxic to the subject. A suitable dosage amount of the composition may be the amount of the composition which is the lowest dosage amount effective for the API to produce a therapeutic and/or prophylactic effect.

The selected dosage amount, form and regime will each depend upon a variety of factors. Such factors may include, for example, the activity of an API, the route of administration, the time of administration, the rate of excretion or metabolism of the API, the rate and extent of absorption, the duration of the treatment, the presence of other drugs, compounds and/or materials used in combination with the API, the age, sex, weight, condition, general health and prior medical history of the subject being treated, and other such factors well known in the medical arts.

The composition may be administered by intramuscular or intravenous injection (encompassing transdermal delivery via a patch), orally (encompassing sublingual administration), intranasally, or by any other suitable route.

Preferably, the composition may be administered by injection, such, for example, as intravenous or intramuscular injection. Optionally when the composition is administered by injection, the subject is monitored for symptoms or signs of a hypersensitivity response, such, for example, as a vaccine-associated hypersensitivity response.

Also preferably, the composition may be administered orally or intranasally. Compositions suitable for oral administration may be presented as discrete dosage forms, especially liquids or aerosol sprays each containing a predetermined amount of the composition. Such dosage forms may be prepared by any of the well-known methods of pharmacy.

The composition may be combined in an intimate admixture with a pharmaceutical carrier, according to conventional pharmaceutical compounding techniques. The carrier may take a wide variety of forms depending on the form of preparation desired for administration. Any of the usual pharmaceutical media may be employed as carriers, such as, for example, one or more of water, oils, and alcohols (encompassing glycols). The forms in which the disclosed pharmaceutical compositions may be incorporated for administration, especially when formulated for administration by injection, orally or intranasally, may include aqueous solutions in saline. The composition may further include one or more pharmaceutically acceptable additives and excipients, such as one or more of the following: detackifiers, anti-foaming agents, buffering agents, polymers, antioxidants, chelating agents, viscomodulators, tonicifiers, odorants, opacifiers, suspending agents, fillers, plasticizers, flavouring agents, preservatives, colouring agents, diluents, binders, disintegrating agents and mixtures thereof.

The prevention or attenuation of the action of microorganisms may be brought about by the inclusion of various antibacterial and antifungal agents, such as one or more of the following: parabens, chlorobutanol, phenol, sorbic acid, thimerosal, and the like.

The composition disclosed herein may be provided in a sterile solution, by incorporating the composition in the required amount in an appropriate solvent (with various other ingredients, where appropriate) by any of the well-known methods of pharmacy. The composition disclosed herein may be provided in a sterile dispersion, by incorporating the composition in the required amount in an appropriate sterile vehicle (with various other ingredients, where appropriate). The composition disclosed herein may be provided as a sterile powder (e.g., for the subsequent preparation of sterile injectable solutions), such as by vacuum-drying and freeze-drying (lyophilisation) techniques which yield a powder of the composition.

Optionally, the composition may be stored before being administered to the subject. The composition may be stored at a temperature in a range of 0° C. or above, especially 4° C. or above, for a period of at least 1 week (optionally, up to 6 months, especially up to 1 year) prior to administering the composition to the subject.

In some embodiments, the liposomal particles can increase the stability of the API during circulation in vivo, especially where the active compound, such as an API, is or comprises nucleic acid such as mRNA. In some embodiments, the particles can protect the API from degradation, especially enzymatic degradation, especially where the API is or comprises nucleic acid such as mRNA. Thus, the d compositions disclosed herein may mitigate or address the problem of how to ensure APIs reach cells once they have been administered to a patient, including how to stabilise APIs while they are circulating in the body.

Meanwhile, a disclosed composition may mitigate or meet the need for tissue or cell targeting, so that an API can be delivered to the correct cells.

Additionally, or alternatively, once a target cell is reached, the disclosed composition may mitigate or solve the challenge of how to ensure efficient API uptake by the cell. For example, the disclosed composition may assist trafficking of the API from the exterior of the cell into the cytoplasm.

Following uptake of an API by a cell, the disclosed composition may mitigate or solve the problem of how to prevent the API degrading too quickly in the cytoplasm. It is thought that the liposomal particles can increase the stability of the API in the cytoplasm of a cell, especially stability against enzymatic degradation, especially where the API is or comprises nucleic acid such as mRNA.

### Relationship Between Aspects of the Invention

It will be understood that a method according to the first aspect of the invention may optionally be used to produce an aqueous suspension of hybrid lipid particles according to a second aspect of the invention and that such a suspension may optionally be used to produce a lyophilized powder of hybrid lipid particles according to the invention. Such products may optionally be used to produce an aqueous suspension of liposomal lipid particles according to the invention. Such an aqueous suspension may optionally be used to produce a lyophilized powder liposomal lipid particles of the invention. An advantage of methods and products of the invention over lipid particles which seek to encapsulate an API or other active ingredient during initial lipid particle formation as described above is that encapsulation methods typically require the encapsulation to take place at the elevated temperature (typically 50 to 70° C.) necessary to form the lipid particles. Such an elevated temperature may degrade heat sensitive active ingredients such as RNA molecules. The present invention allows the hybrid lipid particles to be prepared before the active ingredient to be delivered by the lipid particle is introduced. It also allows the hybrid lipid particles to exist (for example, in aqueous suspension or in lyophilized powder form) for extended periods as an intermediate stockpile or item of commerce for the subsequent production of the final product which may be a liposomal lipid particle.

A lyophilized powder of lipid particles of the invention may be used to produce an aqueous suspension of lipid particles of the invention.

A lyophilized powder of lipid particles of the invention and/or an aqueous suspension of lipid particles of the invention may be used to produce a pharmaceutical composition of the invention.

Hybrid lipid particles of the invention and liposomal lipid particles of the invention may optionally be prepared using a method of the invention.

It will be understood that optional features described or claimed herein as part of one aspect of the invention be understood as optional features, where appropriate or other aspects of the invention.

### EXAMPLES

Various aspects and embodiments of the invention are described below in the following non-limiting examples.

Examples may also describe subject matter which is non-necessarily within the scope of the present invention, but which is included to aid understanding of the invention or to provide a comparison with the invention.

### Methods

A process to manufacture silicon stabilised hybrid Lipid Nano Particles (sshLNP) has been developed using a combination of flow extrusion to form the sshLNP with desired particle size and surface charge properties followed by Tangential Flow Filtration (TFF) to concentrate and purify the solution. To target different tissues, two formulations of sshLNP have been developed, with and without a pegylated lipid.

This process produces an intermediate hybrid lipid particle onto which nucleic acid is loaded immediately prior to the fill-finish operations. Addition of nucleic acid at this stage minimises degradation. This also provides the opportunity for late-stage customisation of sshLNP.

A combination of flow extrusion and TFF provides a highly scalable manufacturing process capable of reliably producing sshLNP at scales of <1 to >100s L making this suitable for use with a range of products from personalised medicines to high volume products.

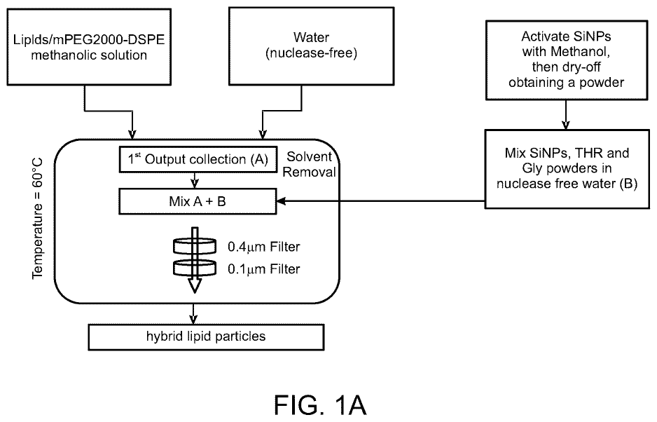

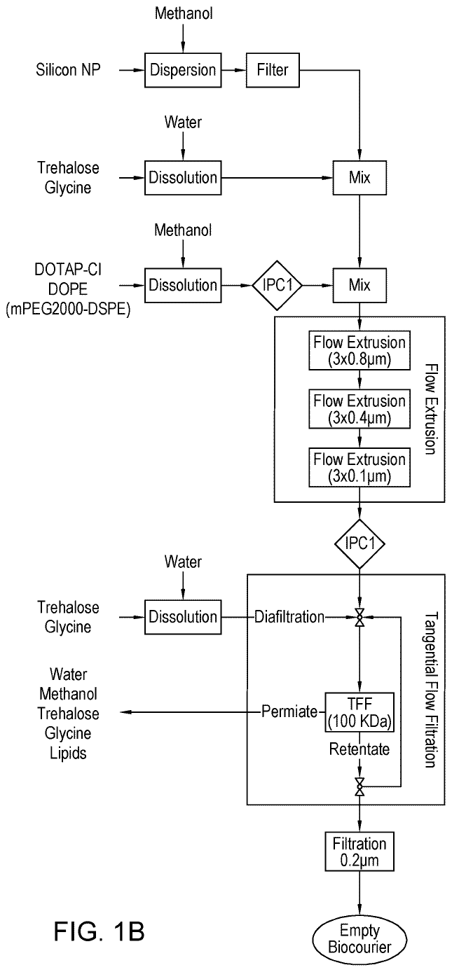

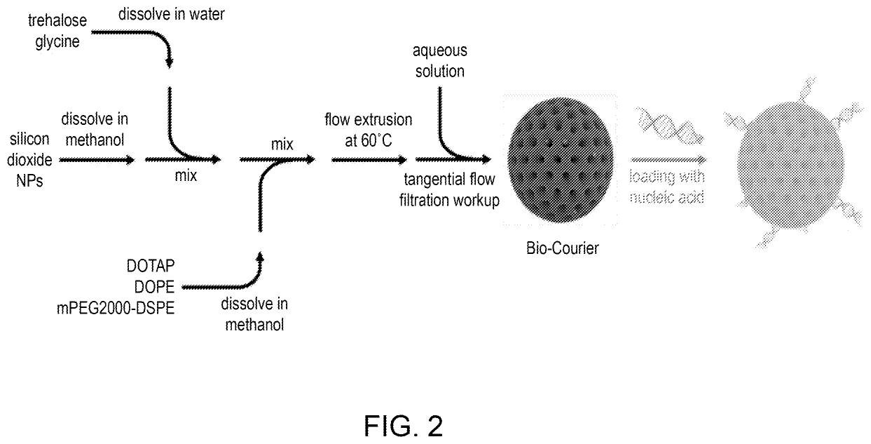

FIG. 1A illustrates an embodiment of the methods of the invention. Silicon nanoparticles (SiNPs) are active by dispersion in methanol as an activating solvent. The optional filtration step may be employed to reduce silicon nanoparticle clumping. The lipids DOTAP-cl, DOPE, and optionally mPEG2000-DSPE are mixed by dissolving them in methanol. An aqueous solution supplemented with trehalose and glycine is first mixed with the SiNP suspension. Into that is injected the dissolved lipid/methanol stream. The resultant mixture is extruded through membranes of decreasing pore size and then subjected to TFF to remove excess trehalose, glycine, methanol and lipid. IPC1 and 2 are quality controls wherein the product is checked against specification.

sshLNP Manufacture
1.1. Biocourier sshLNP Composition

| | Nominal concentration, mg/mL | | | | |
|---|---|---|---|---|---|
| | Pegylated | | Non-pegylated | | |
| | Pre- | | Pre- | | |
| Ingredient | Extrusion | Post-TFF | Extrusion | Post-TFF | Source |
| Silicon | 0.1 | 0.2 | 0.1 | 0.2 | American |
| nanoparticles | | | | | Elements |
| Trehalose | 0.1 | 0.1 | 0.1 | 0.1 | Merck |
| Glycine | 0.05 | 0.05 | 0.05 | 0.05 | Merck |
| DOTAP-C1¹ | 0.725 | 1.45 | 0.775 | 1.55 | Lipoid |
| DOPE² | 0.730 | 1.46 | 0.825 | 1.65 | Lipoid |
| mPEG2000- | 0.145 | 0.29 | Not | Not | Lipoid |
| DSPE³ | | | Present | Present | |
| Nuclease Free | QS | QS | QS | QS | SiSaf |
| Water | | | | | |

¹DOTAP-Cl 1,2-Dioleoyloxy-3-Trimethylammonium Propanchloride, (UNII: 3R78UC794Z, CAS-No. 132172-61-3) 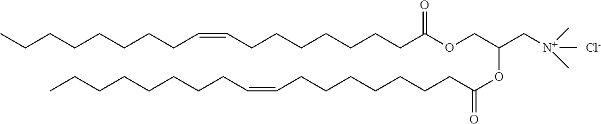 [SMILES: CCCCCCCC/C=C\CCCCCCCC(=O)OCC(C[N+](C)(C)C)OC(=O)CCCCCCC/C=C\CCCCCCCC ; [Cl-]]
²DOPE 1,2-Dioleoyl-sn-glycero-3-phosphoethanolamine, (UNII: JNP6V6A1OU, CAS-No. 4004-05-1) 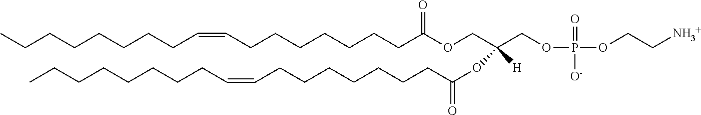 [SMILES: CCCCCCCC/C=C\CCCCCCCC(=O)OC[C@H](COP(=O)([O-])OCC[NH3+])OC(=O)CCCCCCC/C=C\CCCCCCCC]
³mPEG2000-DSPE N-(Carbonyl-methoxypolyethylenglycol-2000)-1,2-distearoyl-sn-glycero-3-phosphoethanolamine, sodium salt, (UNII: 3L6NN8ZZKU, CAS-No. 147867-65-0)

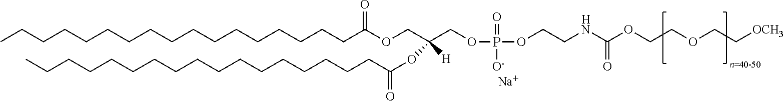

Description of Manufacturing Process of Methanol Injection Combine with TF

Silicon nanoparticles (500 mg) are dispersed in methanol (25 mL) and allowed to stand for 30 minutes with stirring to activate. The dispersion is filtered through a 0.8 μm hydrophilic polyethersulfone filter. Trehalose (400 mg) and glycine (200 mg) are dissolved in Nuclease Free Water (380 ml) and mixed with the dispersion of activated silicon nanoparticles (20 mL) by stirring for 60 min at 50° C.

Either, for Pegylated sshLNP

DOTAP-CI (3500 mg) is dissolved in methanol (350 mL) by stirring at 40° C. for 30 min. DOPE (3500 mg) is dissolved in methanol (350 mL) by stirring at 40° C. for 30 min. mPEG2000-DSPE (650 mg) is dissolved in methanol (65 mL) by stirring at 40° C. for 30 min (IPC 1).

DOTAP-Cl solution (297 mL), DOPE solution (299 mL) and mPEG2000-DSPE solution (59 mL) are mixed. The mixed lipid solution (640 mL) is slowly injected at a flow rate of 10 mL/min into the mixture of silicon nano particles, trehalose and glycine. After addition is complete nuclease free water is added to give a volume of 4 L.

Or, for Non-Pegylated sshLNP

DOTAP-Cl (3500 mg) and is dissolved in methanol (350 mL) by stirring at 40° C. for 30 min. DOPE (3500 mg) is dissolved in methanol (350 mL) by stirring at 40° C. for 30 min (IPC 1).

DOTAP-Cl solution (320 mL) and DOPE solution (340 mL) are mixed. The mixed lipid solution (640 mL) is slowly injected at a flow rate of 10 mL/min into the mixture of silicon nano particles, trehalose and glycine. After addition is complete nuclease free water is added to give a volume of 4 L.

The resulting mixture is extruded over 2 sets of 47 mm polycarbonate extrusion membranes (arranged in parallel to increase effective surface area) of decreasing pore size; 3×0.8 μm, 3×0.4 μm and 3×0.1 μm in each extrusion set. A second extrusion over 3×0.1 μm membranes may be required to obtain the desired physical characteristics (IPC 2).

The extruded mixture is ultrafiltered using Tangential Flow Filtration with 0.5 m² polyethersulfone membranes (100 KDa molecular weight cut-off). After concentration to 2 L volume the mixture is diafiltered using 10 diafiltration volumes (20 L) of a solution containing trehalose (0.1 mg/mL) and glycine (0.05 mg/ml).

After diafiltration the solution is filtered through a 0.2 μm polyethersulfone membrane.

Unlike traditional processing, where the composition of the product is directly proportional to the quantity of material introduced, flow extrusion/TFF is a dynamic process that modifies the content of materials. As developed, the process produces sshLNP with key chemical and physical attributes that result in the effective delivery of nucleic acids.

There are 4 key unit operations in the process

a. Solution Preparation and Mixing

Trehalose and glycine are dissolved in nuclease free water, and the lipids are dissolved in methanol.

Silicon nanoparticles are dispersed in methanol and activated. Activation modifies the contact angle of the surface of the silicon nanoparticles in order to increase their degree of dispersion. Activation also promotes expression of hydroxyl groups on the surface of silicon nanoparticles, thus favouring electrostatic interaction and physical absorption of other excipients onto the surface. The initial silicon nanoparticle dispersion contains 20 mg/mL silicon, using lower concentrations of silicon may affect the physical attributes of the resulting LNPs e.g. average particle size, polydispersity index and zeta potential.

This dispersion is filtered through a 0.8 μm hydrophilic polyethersulfone (PES) membrane filter to remove silicon agglomerates and oversized particles. Without this filtration there is a risk that the extrusion membranes will blind which may affect the physical attributes of the resulting LNPs e.g. average particle size, polydispersity index and zeta potential.

The solution of trehalose and glycine is mixed with the silicon dispersion and the lipid solution is then slowly added to form the lipid nanoparticles. However, at this stage the LNPs have average particle size and polydispersity index greater than the desired values.

Formation of lipid particles by flow injection is preferred over formation of a thin film by evaporation and subsequent rehydration. The thin film/rehydration process have some limitations including difficult to obtaining homogeneous dispersion. Thin film/rehydration methods may also presents challenges for manufacturability at above certain production scales.

b. Flow Extrusion

To reduce average particle size and polydispersity to the desired range the mixture is then extruded through a series of polycarbonate membranes of decreasing pore sizes; 3×0.8 μm, 3×0.4 μm and 3×0.1 μm.

Gradually decreasing pore size of the membranes reduces extrusion pressures and associated mechanical stresses during the process. Using 3 membranes at each stage gives multiple extrusion cycles. This approach of reducing pore size and multiple extrusion cycles produces LNPs with controlled and reproducible physical properties (average particle size, polydispersity index and zeta potential).

During this operation further silicon is removed and some lipid is retained on the membranes.

c. Tangential Flow Filtration (TFF)

TFF using polyethersulfone membranes (100 KDa molecular weight cut-off) performs two functions, ultra-filtration (UF) and diafiltration (DF). Initially the LNP solution is concentrated by UF to reduce volume by 50% and subsequently purified by DF, using 10× diafiltration volumes, to remove methanol and free (unbound) lipids. As trehalose and glycine have low molecular weights these molecules also permeate the membrane. To account for loss of these molecules a diafiltration solution containing both trehalose and glycine is used to maintain the concentration of these materials in the product. TFF has no significant effect on the physical properties of the LNP and does not result in removal of silicon.

d. Final Filtration

At completion of TFF the resulting solution is filtered through a 0.2 μm polyethersulfone filter to remove microbiological contamination and produce sshLNP Biocourier with low bioburden.

This product can be then complexed to mRNA at Room Temperature. In summary, the key product attributes are controlled by the following process operations.

| Key Attributes | Controlling Process Operations |
|---|---|
| Average Particle Size | Flow Extrusion |
| Polydispersity Index | Flow Extrusion |
| Zeta Potential | Flow Extrusion |
| Lipid Content | Flow Extrusion/TFF |
| DOPE | |
| DOTAP-Cl | |
| mPEG2000-DSPE | |
| Methanol | TFF |
| Silicon | Filtration of dispersion/Flow Extrusion |
| Trehalose | TFF |
| Glycine | TFF |
| Bioburden | Final Filtration |
| Bacterial Endotoxin | Input material quality |

### Methods

### Analytical Methods

### In-Flow Extrusion

In-flow extrusion was performed using HPLC pump Knauer K-501 equipped with 50 ml/min stainless steel pump head. Flow rate was set depending on the extrusion volumes in the range between 10 ml/min to 50 ml/min. 0.8 μm, 0.4 μm and 0.1 μm extrusion membranes were used at high temperature of 60° C. using a thermal bath. Holders containing membranes were immersed in water to achieve the required temperature. We investigated the extrusion process by DLS, measuring size and size distribution as well as Zeta potential analysis. The extrusion pressure on the membranes as a function of the time and extruded volume was also monitored. Other investigated parameters for the in-flow extrusion were: extrusion flow rates, number of extrusion membranes, and the use of a pre-filtration step prior to extrusion using syringe filter.

### Tangential Flow Filtration

Tangential Flow Filtration (TFF) was used for the removal of methanol and other impurities such as non-associated lipids and any potential degradation products.

TFF used a TangenX SIUS PD cassette with standard notch offset for 50 ml and 100 ml volumes. Specification of the used cassette:
Part number: XP100LP2L
Membrane type: HyStream
MWCO: 100 kD
Membrane area: (m2) 0.02 m²
Channel type: LP screen channel
Holder configuration L

The micellar lipid particles were two-fold concentrated and washed with 10 diafiltration volumes (DVs) of THR (trehalose) and GLY (glycine) solution with the goal to remove the organic solvent. Note that in this case, diafiltration solution containing THR 0.1 mg/ml and GLY 0.05 mg/ml was used to prevent the loss of these components during TFF. In-process samples were then analyzed post-TFF.

For 2 kg batches, the cassette used has the following specifications (standard notch offset):
Part number: XP100G05L
Membrane type: HyStream
MWCO: 100 kD
Membrane area: (m2) 0.5 m²
Channel type: LP screen channel
Holder configuration: L

The only difference between the two cassette is the membrane area. Larger membranes allow for filtration of larger volumes.

The surface charge, size and size distribution of the samples over all the extrusion steps and post-TFF was assessed by DLS and Zeta potential measurements. Zeta potential, Z-average diameter and PDI are reported for the described experiments. The feasibility of the currently available HPLC method for quantification of lipids after extrusion and post-TFF was investigated using the starting materials as a standard for calibration curves. The removal of methanol was investigated by ¹H-NMR analysis and headspace gas chromatography.

Manufacturing and feasibility studies of in-flow extrusion and TFF are performed under non-GMP conditions and no additional measures for low bioburden processing are employed. The TFF cassette is reused in the manufacture of the various batches after cleaning.

When using Nuclease free water, additional measures in the manufacturing process were employed, such as wearing gloves disinfected with 70% v/v ethanol, routinely cleaning workstation and apparatus using 70% v/v ethanol and the use of sterile lab equipment, sampling in the Laminar flow cabinet.

During extrusion, the solution is passed through Whatman 25 mm polycarbonate membrane filters of defined pore size of 0.4 μm and 0.8 μm. Three membranes were used in a row per each extrusion step. To accomplish extrusion and push the fluid through the membranes, a Knauer K501 HPLC pump equipped with 50 ml/min stainless steel pumphead was used together with a water bath to maintain a working temperature of 60° C. Before each extrusion step, the membranes were washed with MeOH (20 ml) and rinsed with MilliQ water (20 ml). The flow rate used for extrusion was 10 ml/min. Finally, to evaluate the feasibility of the direct addition of SiNP to the aqueous solution and of the extrusion process, and to monitor the features of the SiNP over all the experimental steps, DLS and Zeta potential analysis were performed over all the steps.

### Example 1—for Comparative Purposes, a Description of the Prior Art “Evaporative” Manufacturing Process Used to Produce Reference Values

The “evaporate” manufacturing process of micellar lipid particle production in FIG. 1 starts with dissolving the selected lipids DOTAP, DOPE and mPEG2000-DSPE in methanol. At the same time, mesoporus hydrolysable silicon nanoparticles (SiNPs) were activated by exposure to methanol. Subsequently, the methanol solvent was evaporated in a slow evaporation process to generate the activated SiNPs. This activation step is aimed at rendering the SiNPs amenable to dispersion in water. The activated SiNPs are then dispersed in nuclease-free water in the presence of Trehalose (THR) and Glycine (GLY). An appropriate volume of lipids is successively transferred to a round bottom flask and the methanol evaporated by rotary evaporation. As a result of this evaporation step, a lipid film is generated on the wall of the flask. The suspension containing the dispersed Silicon, THR and GLY is then added to the flask containing the lipid thin film for lipid rehydration.

### Example 2—Use of Membrane Extrusion

Proof of Concept that Membrane Extrusion Methods Work

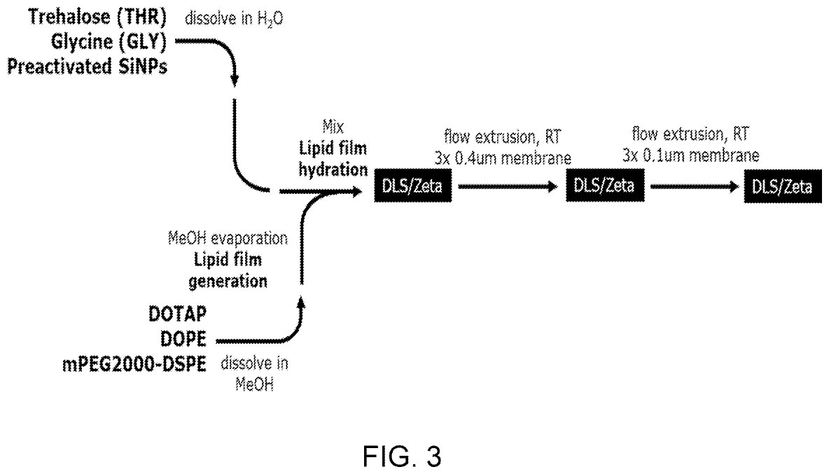

Micellar lipid particles were prepared using a general protocol. Activated SiNPs were mixed with THR and GLY in water and the obtained solution was sonicated for 60 minutes at 50° C. An appropriate volume of the aqueous solution containing NPs-THR-GLY was successively exposed to the lipid film for lipid film hydration. The lipid film is generated by mixing the different methanolic lipid solutions at concentration indicated in the general protocol and evaporating the solvent by rotary evaporation. Preactivated SiNPs were used without any further exposure to MeOH. The general procedure for sample MVI0001 is depicted in FIG. 3.

TABLE 1
Concentration of the SiNP-THR-GLY stock solution

| | |
|---|---|
| [SiNPs]_{fin} | 1 mg/ml |
| [THR]_{fin} | 1 mg/ml |
| [GLY]_{fin} | 0.5 mg/ml |

Lipid solutions of DOTAP-Cl, DOPE and mPEG2000-DSPE were prepared at a concentration of 5 mg/ml by dissolving 25 mg of each lipid in 5 ml of MeOH. The lipid solutions were then sonicated thoroughly for 30 minutes at 40° C. After sonication, the appropriate volume of each lipid solution was transferred to a 10 ml glass round-bottom flask as reported in Table 2.

TABLE 2
Volume of lipid solution to transfer for a batch size of 10 ml

| Size of the batch | 10 ml |
|---|---|
| V DOTAP-C1 | 1.44 ml |
| V DOPE | 1.46 ml |
| V mPEG2000′DSPE | 0.29 ml |

The lipid film was generated by evaporating the solvent by rotary evaporation using the following setup P=300 mbar, T=40° C., t=30 minutes. Next, 1 ml of the aqueous solution containing SiNPs-THR-GLY was exposed to the lipid film for lipid film hydration for 5 min at 60° C. MilliQ water was finally added up to 10 ml. After preparation of the crude sample, in-flow extrusion was performed as described in the first part of this section and reported in FIG. 1. Briefly, the sample was extruded at room temperature through three-in-a-row 25 mm extrusion membranes with a pore size of 0.4 μm and three-in-a-row 25 mm extrusion membranes with a pore size of 0.1 μm using a Knauer K501 HPLC pump at 10 ml/min. Membranes were washed with 20 ml MeOH and rinsed with 20 ml MilliQ water before each extrusion step.

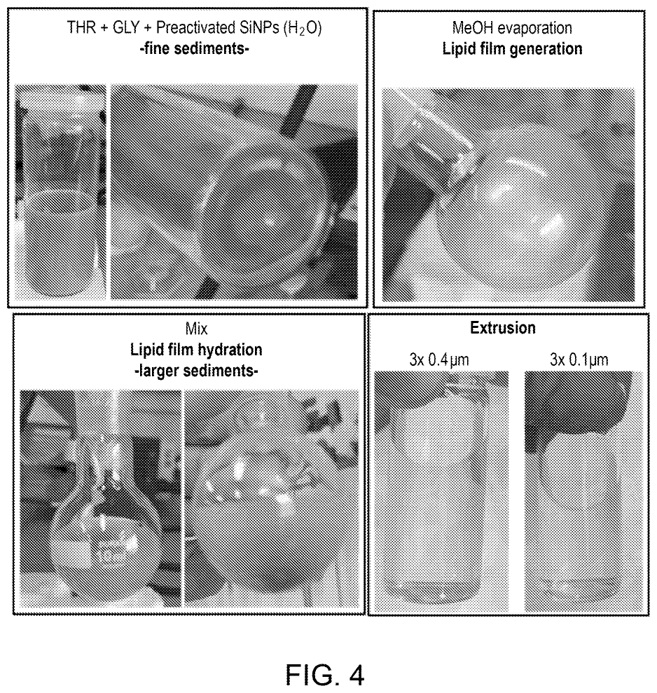

The visual investigation of the obtained solutions at each step is reported in FIG. 4.

The formation of fine sediments in the SiNPs dispersion with THR and GLY was observed. The lipid film was confirmed as correctly generated as indicated by the presence of an opaque film on the wall of the round-bottom flask. When SiNP solution is added to the lipid film, formation of larger sediments was observed. The sediments disappeared when extrusion is performed. Post extrusion, the solutions appeared clear. DLS and Zeta potential analysis of the generated MVI0001 sample were performed before extrusion and after each extrusion step. The obtained values are compared with reference values and are reported in Table 3.

TABLE 3
DLS and Zeta potential investigation of MVI0001
before extrusion and after each extrusion step.
The SiSaf reference values are also reported.

| SAMPLE | Z-Average | | Zeta Potential |
|---|---|---|---|
| MVI0001 | (nm) | PDI | (mV) |
| SiSaf ref. value | 108 +/− 0.50 | 0.089 +/− 0.01 | 39.3 +/− 0.95 |
| Before extrusion | 553.2 +/− 8.22 | 0.362 +/− 0.036 | 57.2 +/− 0.68 |
| 0.4 μm extruded | 190.6 +/− 10.2 | 0.255 +/− 0.019 | 50.4 +/− 1.91 |
| 0.1 μm extruded | 110.8 +/− 0.503 | 0.19 +/− 0.017 | 54.9 +/− 0.27 |

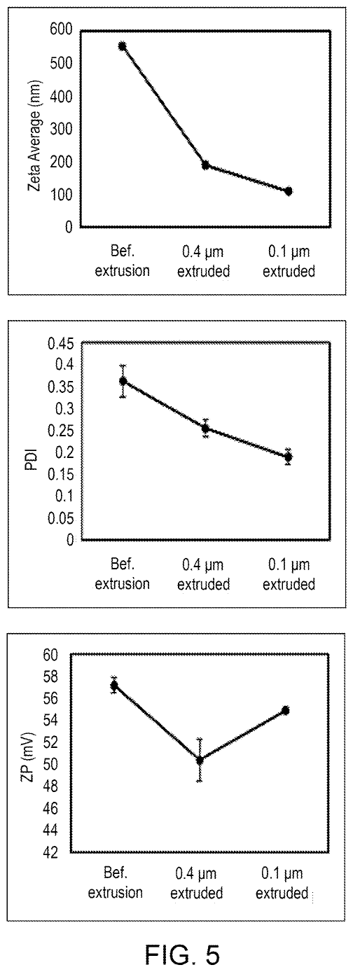

The analysis of the Z-average, PDI and Zeta potential over the extrusion steps is reported in FIG. 5 for MVI0001.

Experimental observations indicated that extrusion improves the monodispersity of the samples. Larger sediments were captured by the 0.4 μm membrane and the solution after 0.1 μm extrusion appeared completely clear. The Zeta potential was largely maintained over all the extrusion steps. Z-average, PDI and Zeta potential values resembled the reference values as indicated by SiSaf reported in Table 3.

### Example 3

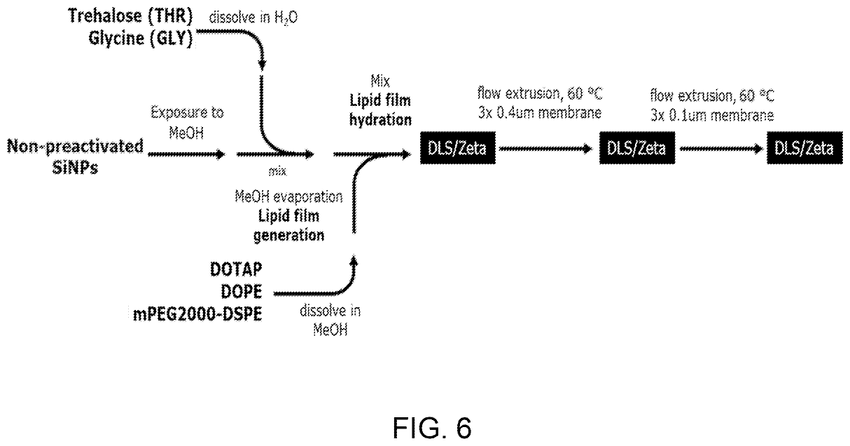

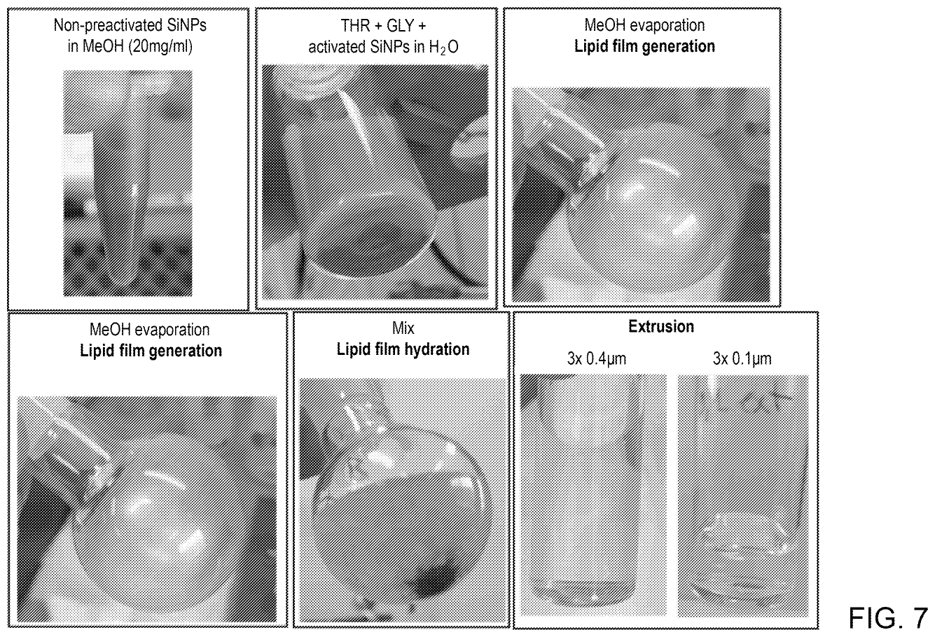

In this experiment the direct addition of non-preactivated SiNPs to the lipid film was investigated. Non-preactivated SiNP were firstly exposed to MeOH for 30 minutes for activation. After activation, SiNPs were directly added to the THR-GLY aqueous solution and sonicated for 60 minutes at 50° C. Finally, the aqueous solution containing SiNPs-THR-GLY was exposed to the lipid film generated by evaporation of MeOH from the methanolic lipid solution. The general procedure for sample MVI0002 is depicted in FIG. 6.

To prepare this sample, 20 mg of non-preactivated SiNPs were initially exposed to 1 ml of MeOH (Table 3).

TABLE 3
Concentration of the SiNP stock solution in MeOH

| | |
|---|---|
| [SiNPs] | 20 mg/ml |

Simultaneously, 20 mg of THR and 10 mg of GLY were weighted in the same tube and 19 ml of MilliQ Water was added to the tube. After 30 minutes of MeOH exposure, SiNPs were considered activated and therefore added to the THR-GLY solution and the solution sonicated 60 minutes at 50° C. Final concentrations of the SiNP-THR-GLY stock solution are the same reported in experiment MVI0001 in Table 1.

Lipid solutions of DOTAP-Cl, DOPE and mPEG2000-DSPE were prepared at a concentration of 5 mg/ml as described in MVI0001 and the appropriate volume of each lipid solution was transferred to a 10 ml glass round-bottom flask as reported in Table 2. Next, the lipid film was generated evaporating the solvent by rotary evaporation using the following setup P=300 mbar, T=40° C., t=30 minutes. Successively, 1 ml of the aqueous solution containing NPs-THR-GLY was exposed to the lipid film for lipid film hydration for 5 min at 60° C. MilliQ water was finally added up to 10 ml.

After preparation of the crude sample, in flow extrusion was performed as reported in FIG. 6 at high temperature of 60° C. over 0.4 μm and 0.1 μm extrusion membranes using Knauer K501 HPLC pump and 10 ml/min as flow rate. Membranes were washed with 20 ml MeOH and rinsed with 20 ml MilliQ water before each extrusion step.

The visual investigation of the obtained solutions at each step is reported in FIG. 7.

In experiment sample MVI0002, SiNPs were initially exposed to MeOH for activation. Large SiNP deposit at the bottom of the cuvette, as depicted in FIG. 7. The formation of sediments in the SiNPs dispersion with THR and GLY was also observed. The lipid film was correctly generated as indicated by the presence of an opaque film on the wall of the round-bottom flask. When the SiNP solution was added to the lipid film, formation of very large sediments and deposit was observed at the bottom of the round-bottom flask. The sediments disappeared when extrusion was performed. Post extrusion, the solutions appeared clear. DLS and Zeta potential analysis of the generated the MVI0002 sample was performed before extrusion and after each extrusion step. The obtained values are compared with SiSaf reference values and are reported in Table 5.

TABLE 5
DLS and Zeta potential investigation of MV10002
before extrusion and after each extrusion step.
The SiSaf reference values are also reported.

| | | | Zeta |
|---|---|---|---|
| SAMPLE | Z-Average | | Potential |
| MVI0002 | (nm) | PDI | (mV) |
| Before extrusion | 979.1 +/− 172.4 | 0.576 +/− 0.109 | 57.1 +/− 1.33 |
| 0.4 μm extruded | 241.8 +/− 1.552 | 0.288 +/− 0.028 | 57.3 +/− 0.85 |
| 0.1 μm extruded | 129 +/− 0.907 | 0.125 +/− 0.012 | 64.9 +/− 2.8 |

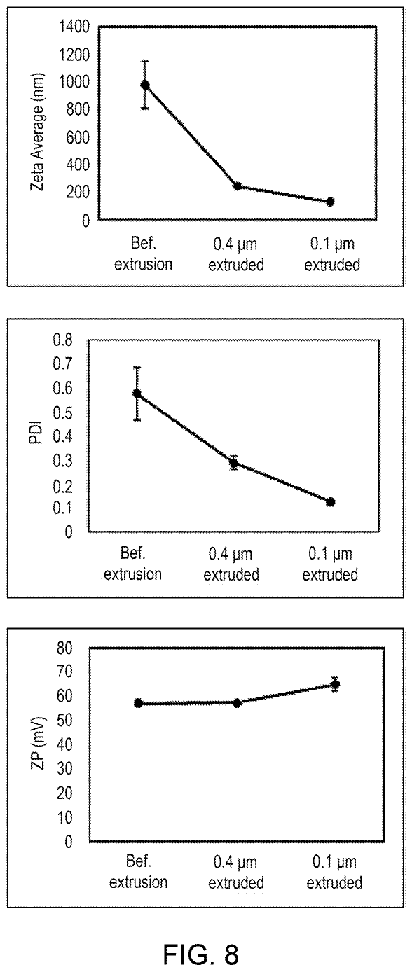

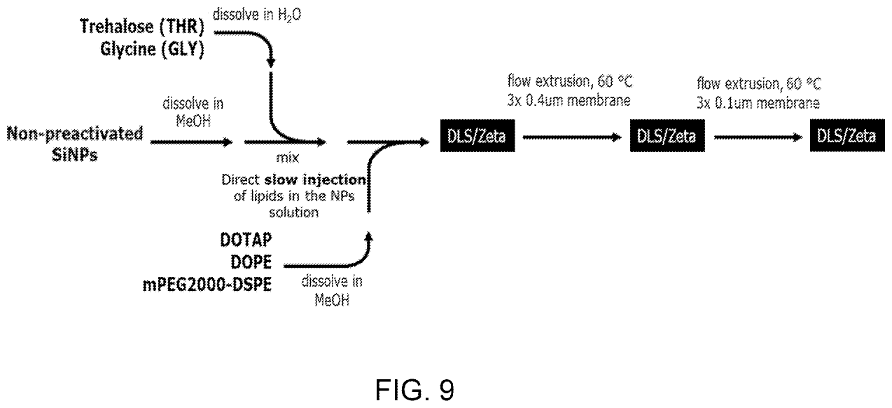

The investigation of the Z-average, PDI and Zeta potential over the extrusion steps is reported in FIG. 8 for sample MVI0002.

In line with experimental observations reported for sample MVI0001, the extrusion improved the monodispersity of the analyzed samples. Larger sediments were captured by the 0.4 μm membrane and the solution after 0.1 μm extrusion appeared completely clear. The Zeta-potential was largely maintained over all the extrusion steps. PDI and Zeta-potential values resembled reference values (Table 5), whilst Z-Average is higher than the reference value.

### Example 4—Elimination of Evaporative Step in Sample MVI0003

The direct injection of lipid solution into the aqueous solution containing SiNPs-THR-GLY was investigated. Non-activated SiNP were first exposed to MeOH for 30 minutes for activation. After activation, SiNPs were directly added to the THR-GLY aqueous solution and sonicated for 60 minutes at 50° C. Finally, the lipids solution was prepared and slowly injected into the aqueous solution containing SiNPs-THR-GLY. The general procedure for MVI0003 is depicted in FIG. 9.

To prepare this sample, SiNPs-THR-GLY solution was prepared as described for sample MVI0002. Briefly, 20 mg of SiNPs were exposed to 1 ml of MeOH for 30 minutes for activation (see Table 4). 20 mg of THR and 10 mg of GLY were weighted in the same tube and 19 ml of MilliQ water was added to the tube. After activation, SiNPs were added to the THR-GLY solution, and the solution was sonicated 60 minutes at 50° C. Final concentrations of the SiNP-THR-GLY stock solution are the same reported in experiment for samples MVI0001 and MVI0002 and in Table 1.

Lipid solutions of DOTAP-Cl, DOPE and mPEG2000-DSPE were prepared at a concentration of 5 mg/ml as described in respect of sample MVI0001 and appropriate volume of each lipid solution as reported in Table 2 was transferred to a 10 ml glass vials. Successively, 1 ml of the aqueous solution containing SiNPs-THR-GLY was transferred to a stirred glass vial. Next, the lipids were collected using a 10 ml sterile syringe and slowly injected in the SiNPs solution using a syringe pump ProSense NE1000. Syringe pump flow rate was set to 3.2 ml/min after setting the correct diameter for the used syringe. Successively, MilliQ water was added up to a final volume of 10 ml. After dilution, the solution was left stirring for 30 minutes to homogenize the solution.

After preparation of the crude sample, in flow extrusion was performed, similarly to the sample MVI0002 experiment and as reported in FIG. 9, at a temperature of 60° C. through 0.4 μm and 0.1 μm extrusion membranes using Knauer K501 HPLC pump at 10 ml/min. Also here, membranes were washed with 20 ml MeOH and rinsed with 20 ml MilliQ water before each extrusion step.

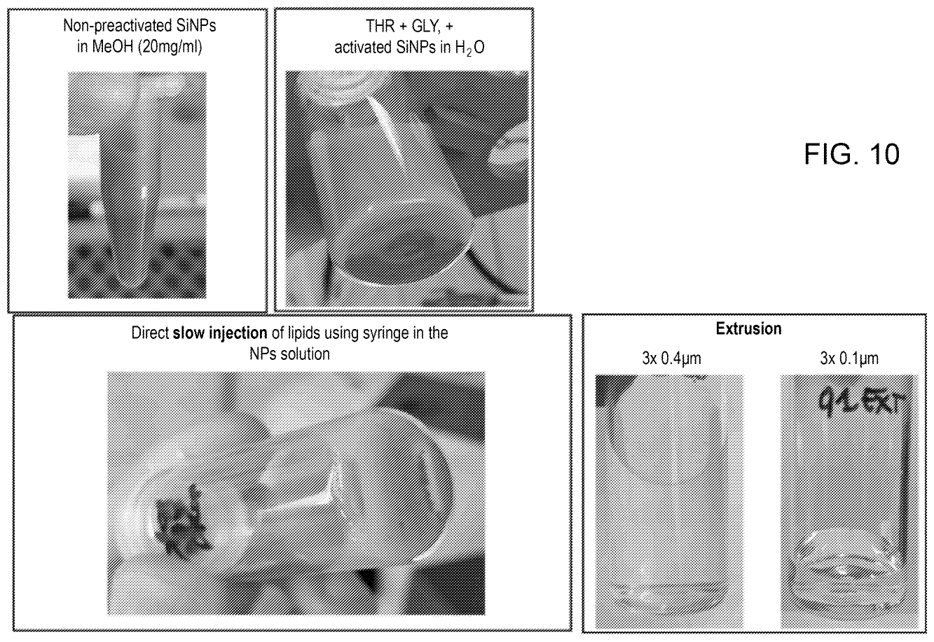

The visual investigation of the obtained solutions at each step is reported in FIG. 10.

In experiment MVI0003, similarly to experiment MVI0002, SiNPs were initially exposed to MeOH for activation. SiNP deposits at the bottom of the cuvette, as depicted in FIG. 10 were observed. The formation of large sediments in the SiNPs dispersion in the presence of THR and GLY were also observed. When lipids were added to the SiNPs solution, the formation of sediments was observed as well. However, the direct slow injection of lipids using a syringe in the SiNPs solution seems to improve SiNP solubility, and less sedimentation with respect to the sedimentation was observed compared to that of MVI0002 (FIG. 7). The sediments disappeared when extrusion was performed. Post extrusion, the solutions appeared clear. DLS and Zeta-potential analysis of the MVI0003 sample were performed before extrusion and after each extrusion step. The obtained values were compared with SiSaf reference values and are reported in Table 6.

TABLE 6
DLS and Zeta potential investigation of MVI0003 before extrusion and
after each extrusion step. The SiSaf reference are also reported.

| | | | Zeta |
|---|---|---|---|
| SAMPLE | Z-Average | | Potential |
| MVI0002 | (nm) | PDI | (mV) |
| SiSaf ref. value | 108 +/− 0.50 | 0.089 +/− 0.01 | 39.3 +/− 0.95 |
| Before extrusion | 449.3 +/− 10.62 | 0.335 +/− 0.024 | 61.4 +/− 2.17 |
| 0.4 μm extruded | 148.8 +/− 1.626 | 0139 +/− 0.017 | 66.3 +/− 2.64 |
| 0.1 μm extruded | 102.1 +/− 0.173 | 0.135 +/− 0.009 | 56 +/− 0.808 |

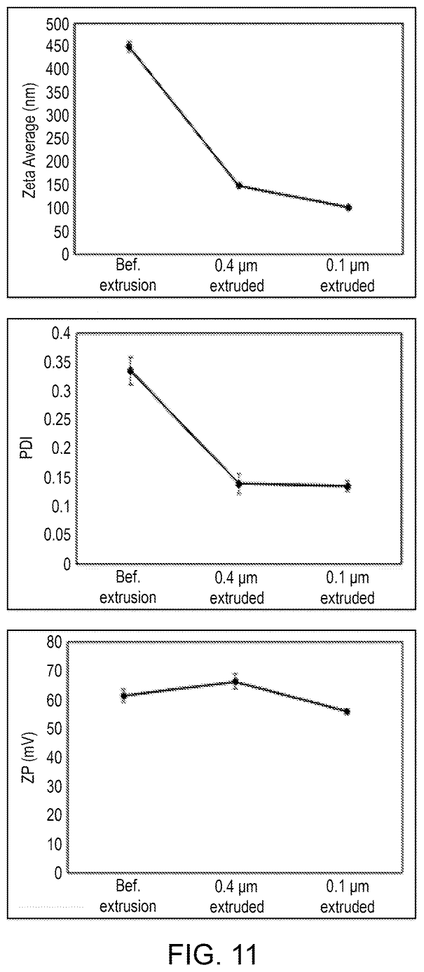

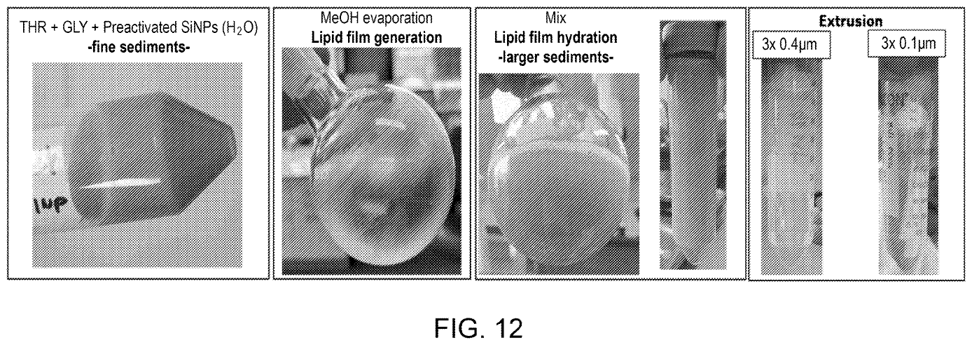

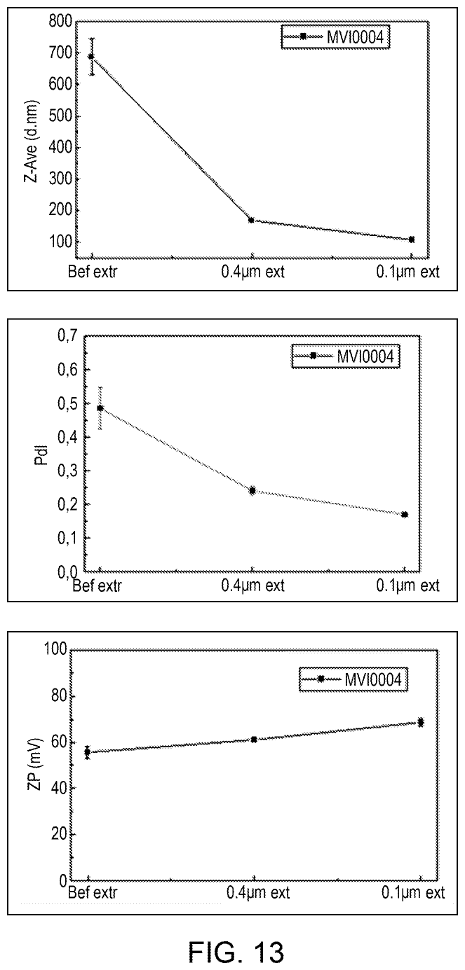

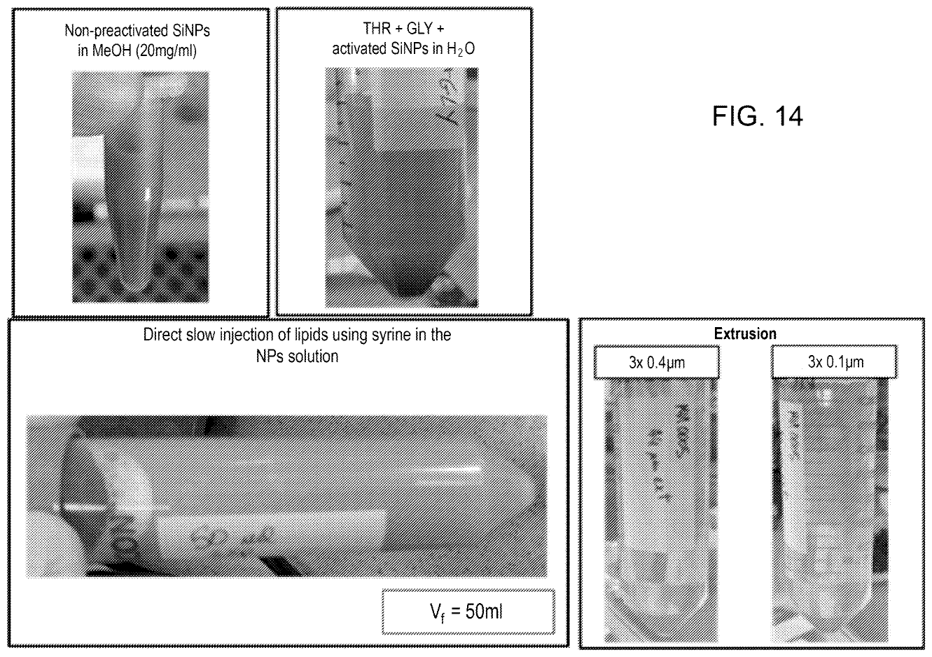

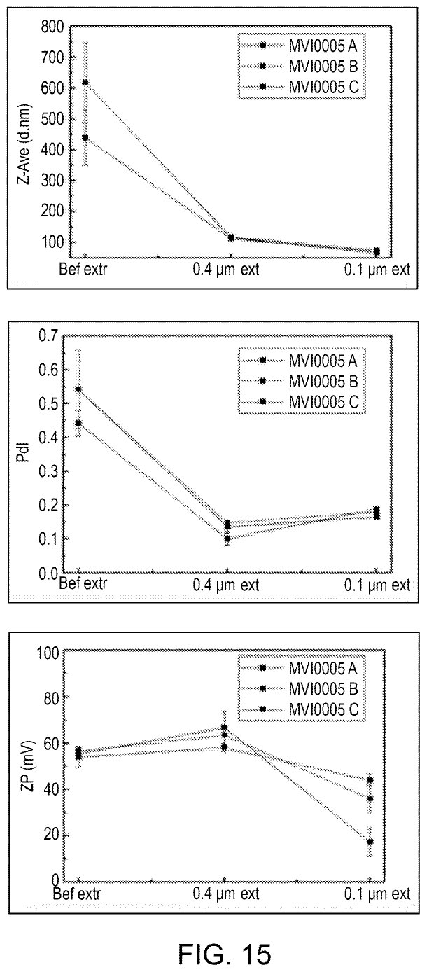

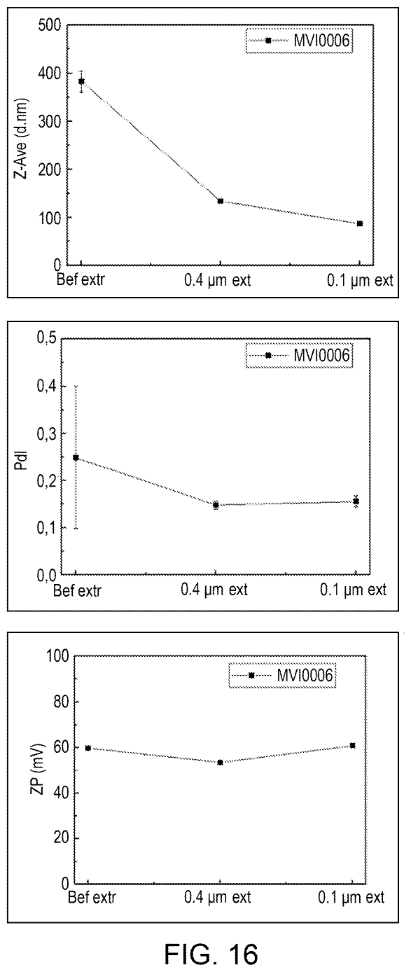

The analysis of the Z-average, PDI and Zeta-potential over the extrusion steps is reported in FIG. 11 for MVI0003.

Once again, extrusion improves monodispersity of the MVI0003 sample. Larger sediments were captured by the 0.4 μm membrane and the solution after 0.1 m extrusion appeared completely clear. The Zeta-potential was largely maintained over all the extrusion steps. Z-average, PDI and Zeta-potential values resembled reference values, as indicated in Table 6.

In Table 7, the DLS features of all the three analyzed samples after 0.1 μm extrusion are reported.

TABLE 7
Comparison of the DLS features of the three investigated experimental procedures

| | | | Z- | | ZETA |
|---|---|---|---|---|---|
| | | | AVERAGE | | POTENTIAL |
| EXPERIMENT | | VOL | (NM) | PDI | (MV) |
| Sample ref. value | | | 108 +/− 0.50 | 0.089 +/− 0.01 | 39.3 +/− 0.95 |
| 01 | prehydrated + film | 10 ml | 110 +/− 0.50 | 0.190 +/− 0.02 | 54.9 +/− 0.27 |
| | hydration | | | | |
| 02 | non-prehydrated + film | 10 ml | 129 +/− 0.91 | 0.125 +/− 0.02 | 64.9 +/− 2.80 |
| | hydration | | | | |
| 03 | non-prehydrated + | 10 ml | 102 +/− 0.17 | 0.135 +/− 0.01 | 56.0 +/− 0.81 |
| | injection | | | | |

Observations on sample MVI0001 and sample MVI0003 indicate that DLS features of the samples after extrusion are in line with the reference values. Sample MVI0001 also visually resembles the expected outcome, while sample MVI0003 is characterized by the presence of large aggregates in the SiNPs solution. These observations already suggest that the evaporative procedure has been successfully reproduced in trial MVI0001, and that the direct injection of lipid into non-preactivated SiNPs solution is feasible. It is important to note that this last experimental approach is the most suitable for scale-up of the manufacture process of the micellar lipid particles.

### Example 5—Optimization of in-Flow Extrusion

The in-flow extrusion process was further investigated by evaluating:
1. the use of an additional 0.8 μm membrane to capture large silicon aggregates (MVI0007)
2. the reproducibility of experimental results using nuclease free water (MVI0008)
3. the use of a prefiltration step prior to in-flow extrusion using 0.8 μm syringe filter (MVI0011)
4. the extrusion of a large (1 L) volume of micellar lipid particle solution—(MVI0012)
5. the extrusion pressure generated by the different experimental setups

Representative results are reported for each experiment.

### Exploring the Use of an Additional 0.8 μm Extrusion Membrane to Capture Large Aggregates (MVI0007)

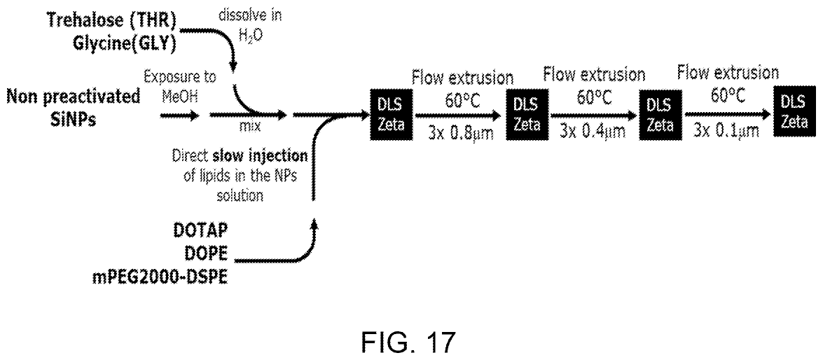

The use of an additional extrusion membrane with a pore size of 0.8 μm was investigated with the goal to remove large SiNP aggregates. Those aggregates can indeed generate the experimental variability observed in the reported experiments. The experimental setup for sample MVI0007 using the additional 0.8 μm membrane is reported in FIG. 17.

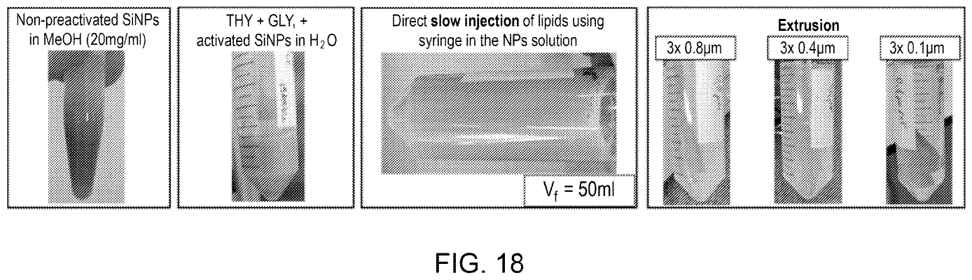

The micellar lipid particles were prepared in FIG. 17 at final volume of 50 ml. In flow extrusion through three-in-a-row 25 mm extrusion membranes with a pore size of 0.8 μm, three-in-a-row 25 mm extrusion membranes with pore size of 0.4 μm and three-in-a-row 25 mm extrusion membranes with pore size of 0.1 μm was performed at 60° C. The visual investigation of the solution obtained at each step for experiment MVI0007 is reported in FIG. 18.

In experiment MVI0007, the formation of sediments in the SiNPs dispersion with THR and GLY were observed. When lipids are added to the SiNPs solution, the formation of sediments was also observed. The sediments disappeared when extrusion was performed. All post-extrusion solutions appeared clear.

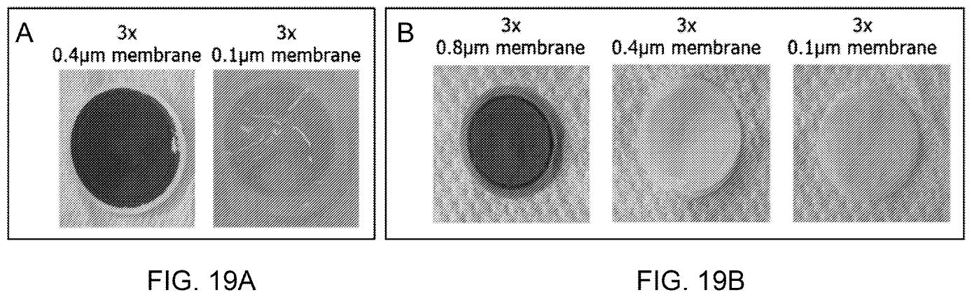

For comparison, in FIG. 19A the pictures of the 0.4 μm and 0.1 μm membranes after extrusion without the use of a 0.8 μm membrane are reported. FIG. 19B reports the pictures of the 0.8 μm, 0.4 μm and 0.1 μm membranes after extrusion.

In FIG. 19A, the 0.4 μm extrusion membrane appeared completely covered by a brown layer of SiNPs already indicating that, in the absence of the 0.8 μm membrane, that membrane captured most of the aggregates. Oppositely, as indicated in FIG. 19B, the presence of the 0.8 μm extrusion membrane captured most of the aggregates of the solution, as it appeared covered by a brown layer of SiNPs.

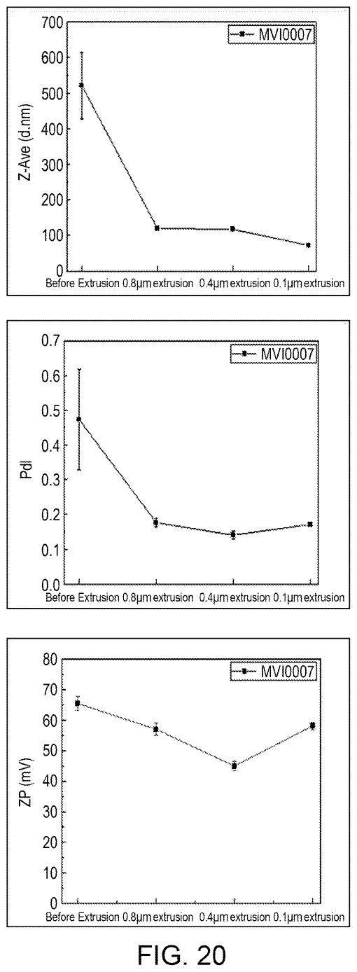

DLS and Zeta potential analysis of the MVI0007 sample were performed before extrusion and after each extrusion step. The results of the DLS investigation on the sample is reported in Table 8 and in FIG. 20.

TABLE 8
DLS and Zeta potential investigation of sample MVI0007 before extrusion
and after each extrusion step. The reference values are also reported

| | | | Zeta |
|---|---|---|---|
| SAMPLE | Z-Average | | Potential |
| MVI0002 | (nm) | PDI | (mV) |
| Before extrusion | 521.17 +/− 94.1 | 0.47 +/− 0.144 | 65.6 +/− 2.25 |
| 0.8 μm extruded | 119.9 +/− 1.8 | 0.18 +/− 0.012 | 57.13 +/− 2.06 |
| 0.4 μm extruded | 116.53 +/− 72.1 | 0.14 +/− 0.013 | 45.1 +/− 1.61 |
| 0.1 μm extruded | 72.11 +/− 0.86 | 0.17 +/− 0.002 | 58.233 +/− 1.16 |

DLS investigation confirmed that the 0.8 μm extrusion membrane removed most of the aggregates of SiNPs, as indicated by the drop of the Z-average value post 0.8 μm extrusion.

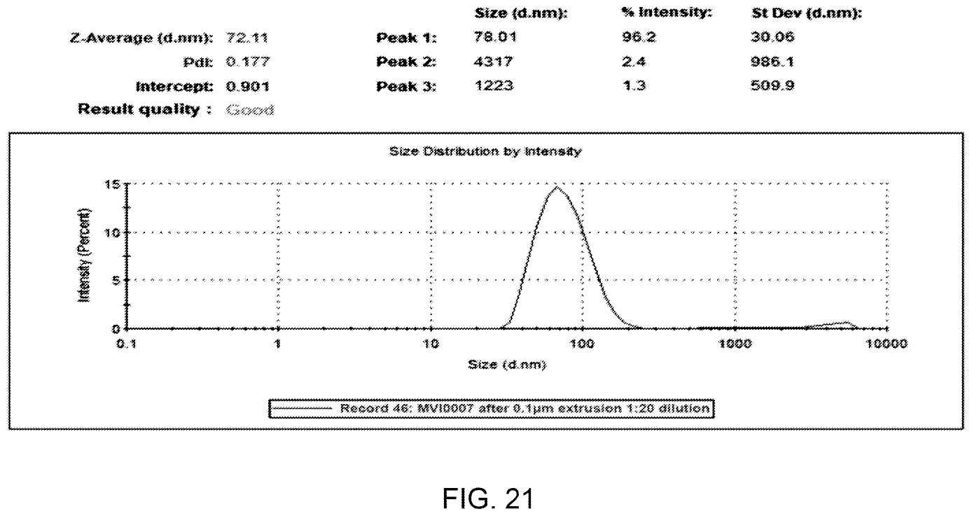

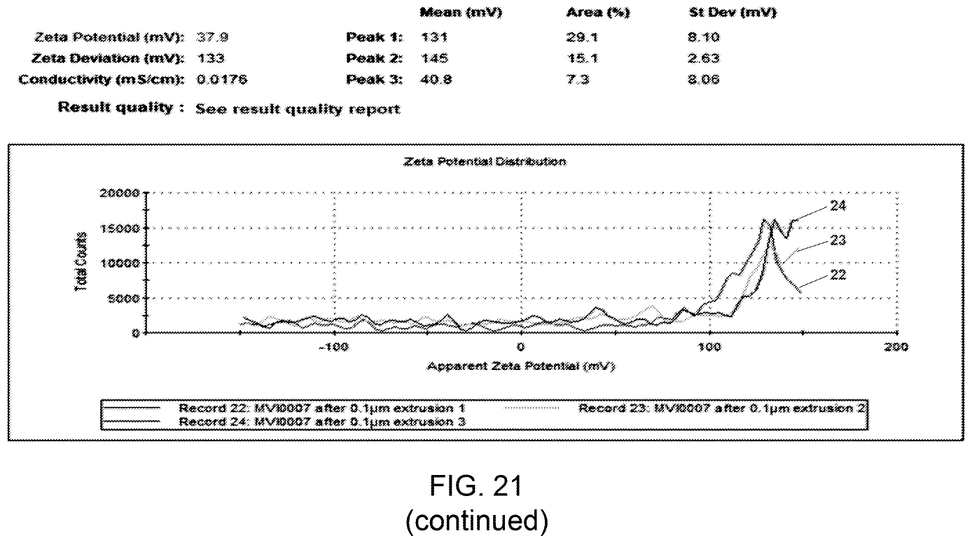

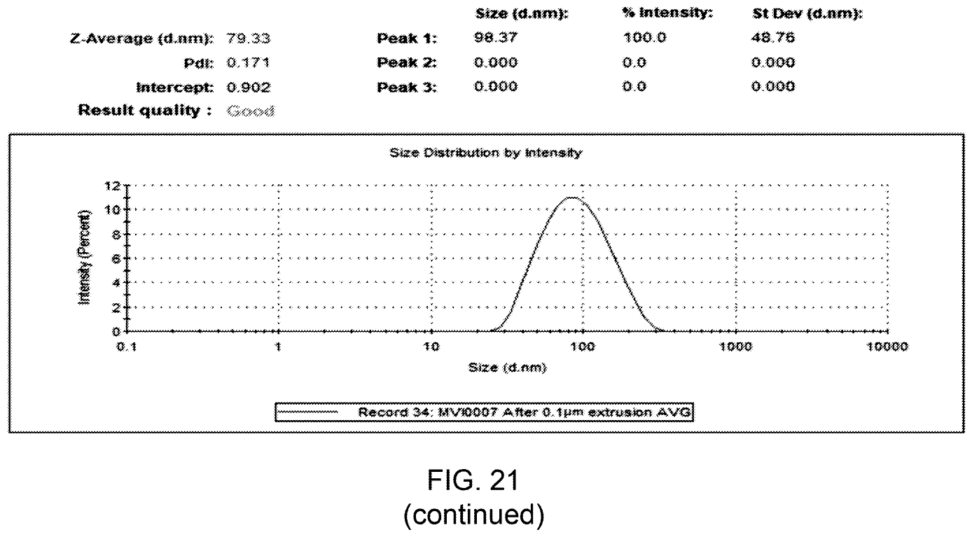

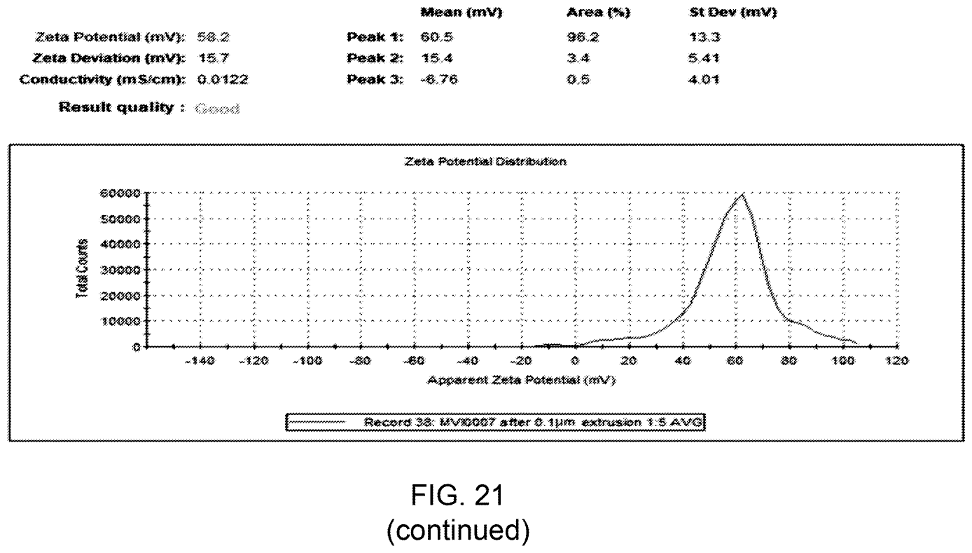

The Zeta potential measurements after 0.1 μm extrusion showed relatively low counts. For this reason, more concentrated samples were analysed to achieve good Zeta potential values. The investigated dilution factors were 20× (which is the value indicated by SiSaf) and 5×. The result of this investigation is reported in FIG. 21.

FIG. 21 shows that the use of a 5× dilution factor successfully increased the quality of the Zeta potential signal. Note that the reference dilution value is 20×. Interestingly, the Z-average diameter using 20× dilution factor is 72 nm, whilst the Z-average diameter obtained using 5× dilution factor is slightly higher (80 nm). The reference value is 108 nm.

### Example 6—Reduction in Process Steps

In this example the use of nuclease-free water for the generation of a 50 ml batch of micellar lipid particles was investigated. The experimental setup used in this experiment is the same as in FIG. 17 and used for sample MVI0007. Note that in this case the experimental procedure was slightly changed to decrease the exposure time of SiNPs to non-sterile environment and avoid unnecessary steps which can potentially lead to microbial contamination. Thus, after activation, SiNPs were diluted by direct addition of the THR-GLY aqueous solution in the same tube where the SiNPs were activated, instead of transferring SiNPs in the tube with the THR-GLY aqueous solution. In-flow extrusion through 0.8 μm, 0.4 μm and 0.1 μm membranes was performed at 60° C.

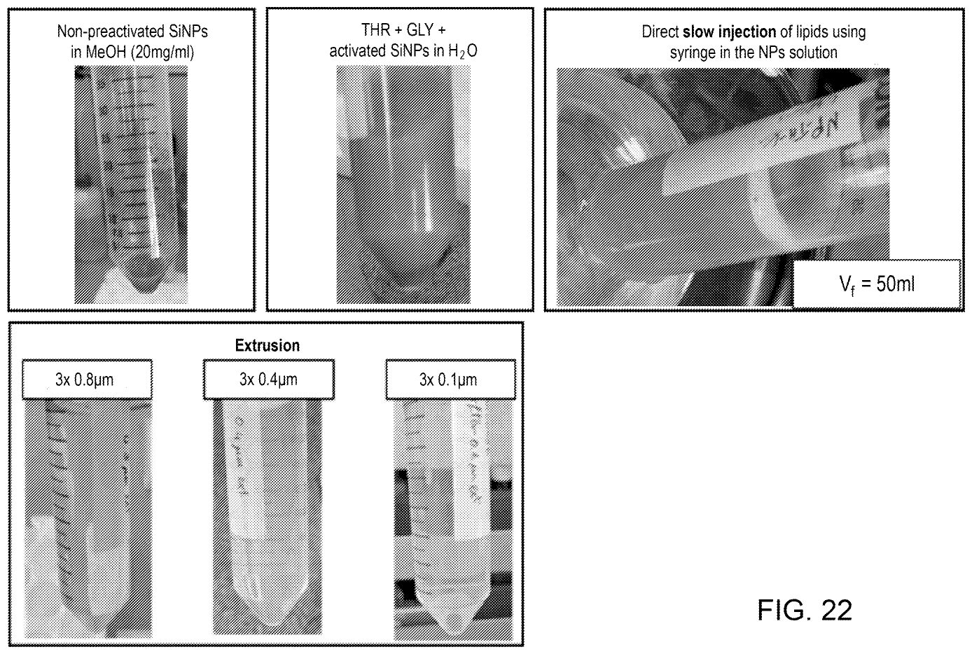

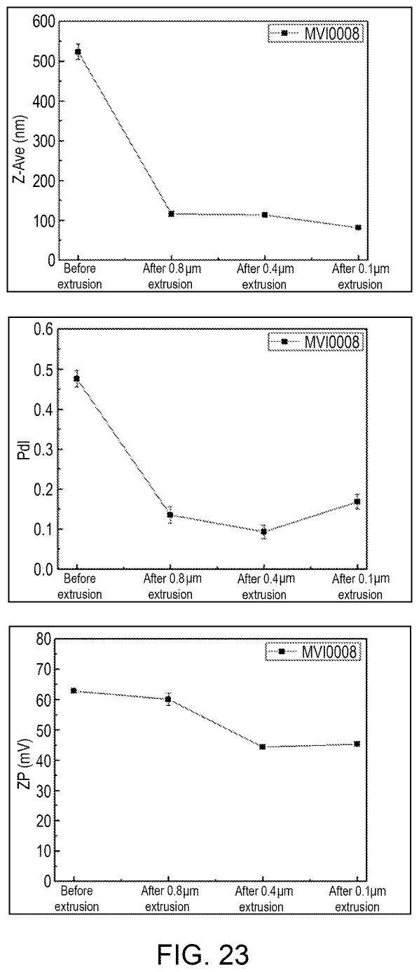

In FIG. 22 pictures of the obtained solution for labelled sample MVI0008 are reported.

The formation of substantial sediments is still observed in experiment MVI0008 before extrusion as indicated in FIG. 22. DLS analysis of the MVI0008 sample was performed before extrusion and after each extrusion step. The results of the DLS investigation on the three samples is reported in Table 9 and in FIG. 23.

TABLE 9
DLS and Zeta potential investigation of Sample
08 before extrusion and after each extrusion step.
The SiSaf reference values are also reported

| | | | Zeta |
|---|---|---|---|
| SAMPLE | Z-Average | | Potential |
| MVI0002 | (nm) | PDI | (mV) |
| Before extrusion | 523.9 +/− 20.4 | 0.476 +/− 0.02 | 62.87 +/− 0.60 |
| 0.8 μm extruded | 116.3 +/− 1.04 | 0.136 +/− 0.02 | 60.13 +/− 2.02 |
| 0.4 μm extruded | 114 +/− 2.25 | 0.093 +/− 0.017 | 44.4 +/− 0.61 |
| 0.1 μm extruded | 74.67 +/− 1.18 | 0.148 +/− 0.012 | 45.4 +/− 0.115 |

In this experiment, micellar lipid particles were prepared using sample MVI0007 as reference experiment and nuclease-free water. Experimental observations indicated that experiment MVI0008 successfully reproduced MVI0007 and therefore the expected features using nuclease-free water. Sediments were largely captured by the 0.8 μm membrane. Extrusion improved the monodispersity of all the analyzed samples. The solution after 0.1 μm extrusion appeared completely clear. Importantly, the modification of the order of addition did not affect the experimental results.

### Example 7—Use of Optional Prefiltration Step

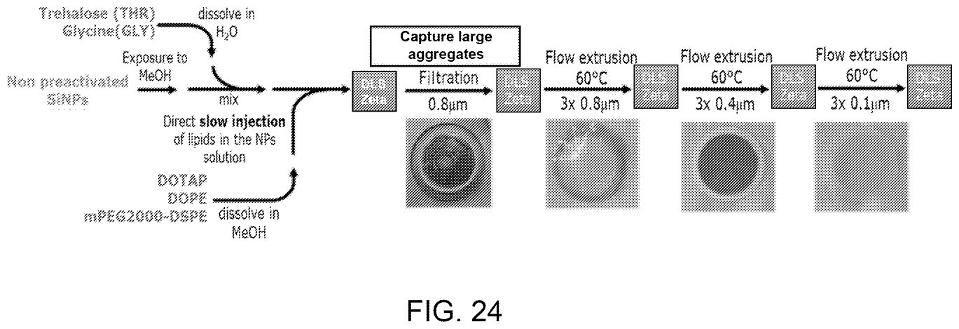

In this example, the use of a prefiltration step prior to in flow extrusion to filter out large aggregates from the SiNPs solution, was investigated. The final volume of this experiment was increased to 100 ml. Moreover, MeOH lipid stock solutions were prepared at increased concentration of 10 mg/ml (instead of 5 mg/ml) to reduce the volume of MeOH injected with the lipids in the final solution. For prefiltration, we used a 0.8 μm hydrophilic polyethersulfone syringe filter. FIG. 24 shows the strategy used in this experiment, which was named MVI0011.

Micellar lipid particles were filtered using 0.8 μm hydrophilic polyethersulfone syringe filter before the in-flow extrusion was performed. The picture of the syringe filter reported in FIG. 24 already shows that the prefiltration step captured most of the large aggregates on the SiNPs solution, as indicated by the brownish layer in the syringe filter and by the absence of the same layer on the 0.8 μm extrusion membrane. Conversely, in experiments where this prefiltration step was not performed, the 0.8 μm membrane appeared completely covered by that brownish layer of SiNPs (see FIG. 19).

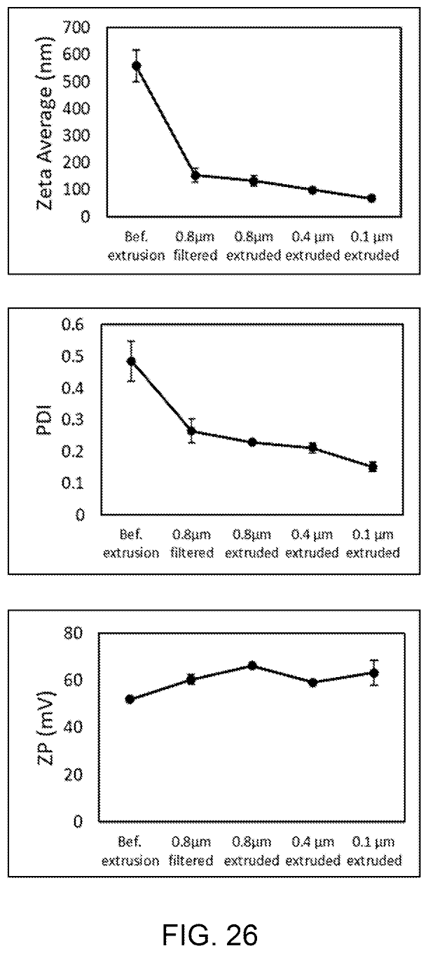

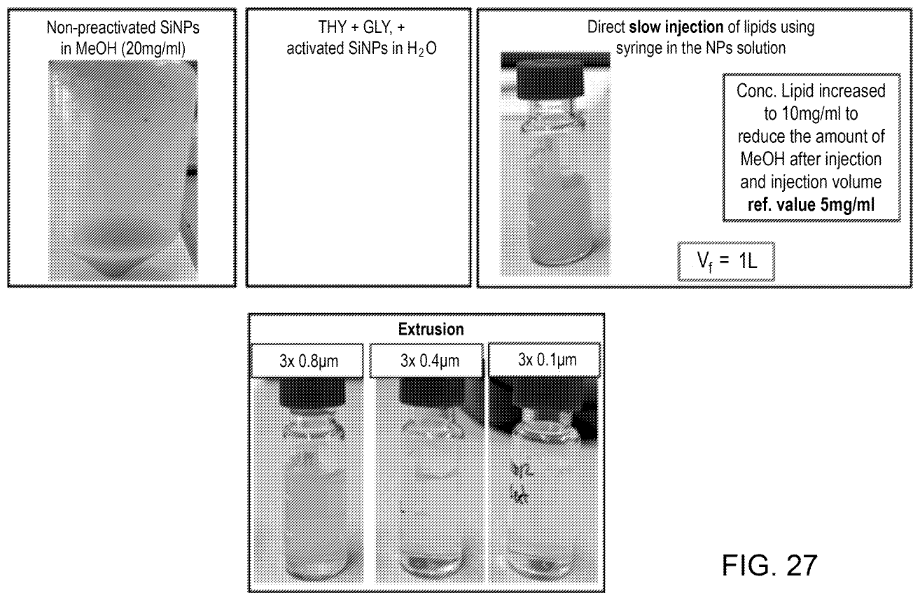

DLS analysis of the MVI0011 sample was performed after each prefiltration and extrusion step. The results of the DLS and Zeta potential investigation on the sample is reported in Table 10 and FIG. 26.

TABLE 10
DLS and Zeta potential investigation of MVI0011 before extrusion and after
each extrusion step. The SiSaf reference values are also reported

| | | | Zeta |
|---|---|---|---|
| SAMPLE | Z-Average | | Potential |
| MVI0002 | (nm) | PDI | (mV) |
| Before extrusion | 558.6 +/− 58.98 | 0.486 +/− 0.063 | 52 +/− 0.089 |
| 0.8 μm filtered | 154.6 +/− 25.5 | 0.266 +/− 0.004 | 60.4 +/− 2.11 |
| 0.8 μm extruded | 133.5 +/− 19.37 | 0.231 +/− 0.008 | 66.4 +/− 0.69 |
| 0.4 μm extruded | 100.3 +/− 3.47 | 0.213 +/− 0.016 | 59.2 +/− 0.76 |
| 0.1 μm extruded | 68.62 +/− 0.82 | 0.153 +/− 0.014 | 63.3 +/− 5.2 |

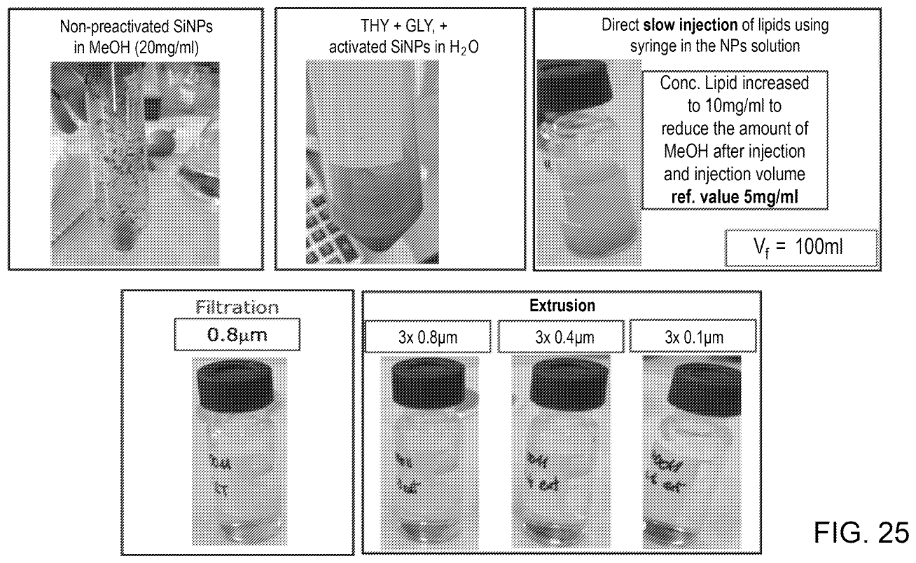

The Z-average value improved after filtration, indicating the removal of large aggregates from SiNPs solution, accordingly with visual observations reported in FIG. 25. DLS measurements in sample MVI0011 were in line with the expected values after extrusion.

Finally, the use of a more concentrated stock lipid solution has also been investigated. The increased MeOH concentration did not affect DLS and Zeta potential features of the micellar lipid particles.

### Example 8—Demonstration of Large Batch

In this example the production of micellar lipid particles at final volume of 1 L is explored, to demonstrate the feasibility of the extrusion process for large volumes batches. This is considered as an intermediate step before production. In this experiment, which is named MVI0012, the use of extrusion membranes with diameter of 47 mm was explored.

For preparation of the micellar lipid particles, the experimental setup reported in FIG. 17 was used adjusting the volumes to a final batch size of 1 L. Briefly aqueous SiNPs-THR-GLY solution was prepared using the standard procedure of SiNP activation in MeOH followed by SiNP dilution in aqueous THR-GLY solution. To do that, 100 mg of non-preactivated SiNP were weighted and transferred to a 200 ml sterile Falcon tube and 5 ml of MeOH was added for activation. THR-GLY aqueous solution was prepared by weighing 100 mg of THR and 50 mg of GLY and adding 95 ml of MilliQ water. After SiNP activation 30 minutes at room temperature, the THR-GLY solution was added to the SiNP and sonicated 60 min at 50° C.

Next, 10 mg/ml stock solution of DOTAP-Cl, DOPE and mPEG2000-DSPE were prepared by weighing 750 mg of DOTAP-Cl, 750 mg of DOPE and 200 mg of mPEG2000-DSPE and adding 75 ml of MeOH for solubilizing the DOTAP-Cl and DOPE and 20 ml of MeOH for solubilizing mPEG2000-DSPE. Lipid solutions were next sonicated 30 minutes at 40° C. 100 ml of the SiNPs-THR-GLY solution were transferred to a 1 L sterile bottle. The volume of each lipid solution to inject in the SiNPs solution at this scale is reported in Table 11. Due to high the volume of the mixed lipid solution, this was slowly injected using a HPLC pump. The flow rate used for addition was 6 ml/min. Finally, MilliQ water was added up to 1 L.

TABLE 11
Volume of lipid solution to transfer for a batch size of 1000 ml

| | | |
|---|---|---|
| Size of the batch | 1000 | ml |
| V DOTAP-C1 | 72.5 | ml |
| V DOPE | 73 | ml |
| V mPEG2000′DSPE | 14.5 | ml |

In flow extrusion through 0.8 μm, 0.4 μm and 0.1 μm membranes was performed at 60° C. In FIG. 27 pictures of the obtained solution are reported.

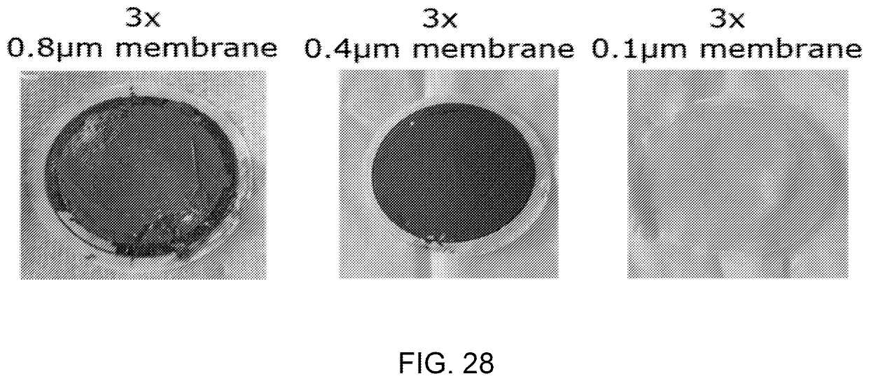

Interestingly and differently from other experiments, the solution after 0.8 μm extrusion appeared yellow, suggesting that some of the silicon passed through the membrane. This is supported by the visual investigation of the membranes after extrusion reported in FIG. 28 by the presence of the brown layer on the 0.4 μm membranes, which was not observed in other experiments.

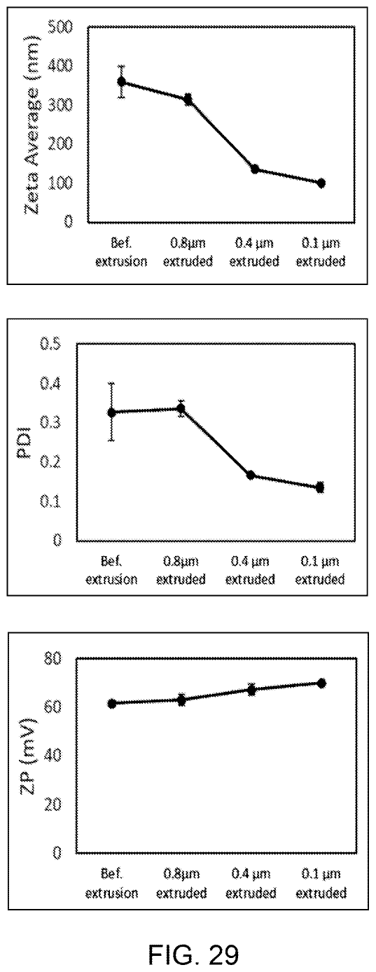

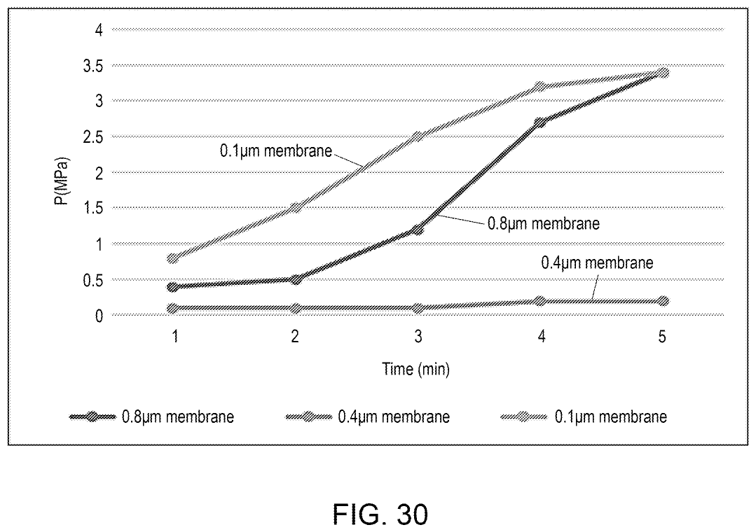

The results of the DLS investigation on the sample are reported in Table 12 and in FIG. 29.

TABLE 12
DLS and Zeta potential investigation of MVI0012

| | | | Zeta |
|---|---|---|---|
| SAMPLE | Z-Average | | Potential |
| MVI0002 | (nm) | PDI | (mV) |
| Before extrusion | 359.8 +/− 39.75 | 0.327 +/− 0.073 | 61.5 +/− 1.1 |
| 0.8 μm extruded | 314.5 +/− 14.25 | 0.336 +/− 0.02 | 63 +/− 2.15 |
| 0.4 μm extruded | 136.6 +/− 0.61 | 0.166 +/− 0.001 | 67.2 +/− 2.11 |
| 0.1 μm extruded | 100.9 +/− 1.059 | 0.135 +/− 0.012 | 69.9 +/− 0.61 |

Experimental observation on experiment MVI0012, indicated that DLS and Zeta potential measurements are in line with the reference values after extrusion. Moreover, the use of 47 mm membranes decreased the extrusion pressure, whilst increasing the lipid concentration in the initial MeOH lipid solution decreased the amount of MeOH in the final solution.

### Example 9—Analysis by HPLC

In this section the development of a method for HPLC-CAD quantification of the lipid content in the solution is investigated. To do that, we investigated the use of Waters Xbridge Phenyl column (SKU: 186003352):
Particle size: 5 μm
size: 130 Å
Inner diameter 4.6 mm
Length 150 mm

We used ammonium-acetate at concentration 40 mM vs. Methanol as elution buffers, with gradient profiles reported in Table 13.

TABLE 13
Gradient profile of the used eluents

| Time | % Ammonium-acetate | % Methanol |
|---|---|---|
| 00.0 min | 35% | 65% |
| 02.0 min | 35% | 65% |
| 10.0 min | 5% | 95% |
| 20.0 min | 5% | 95% |
| 25.0 min | 35% | 65% |
| 30.0 min | 35% | 65% |

1 mg/mL solutions of each lipid were then prepared and injected separately on the phenyl column.

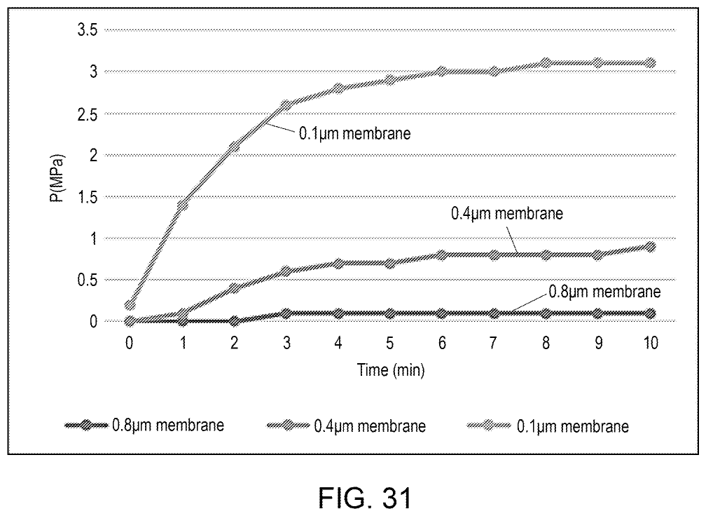

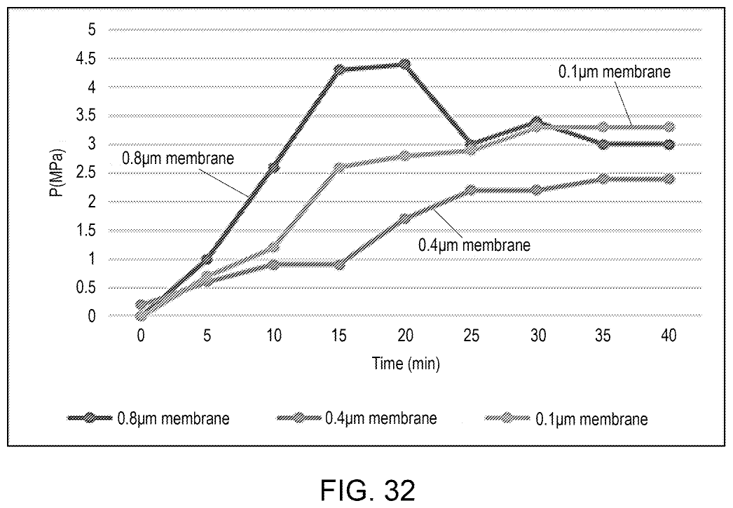

The 1:1:1 mix of all three lipid solutions was also prepared and investigated using the same method. The chromatogram of the mix solution is reported in FIG. 31.

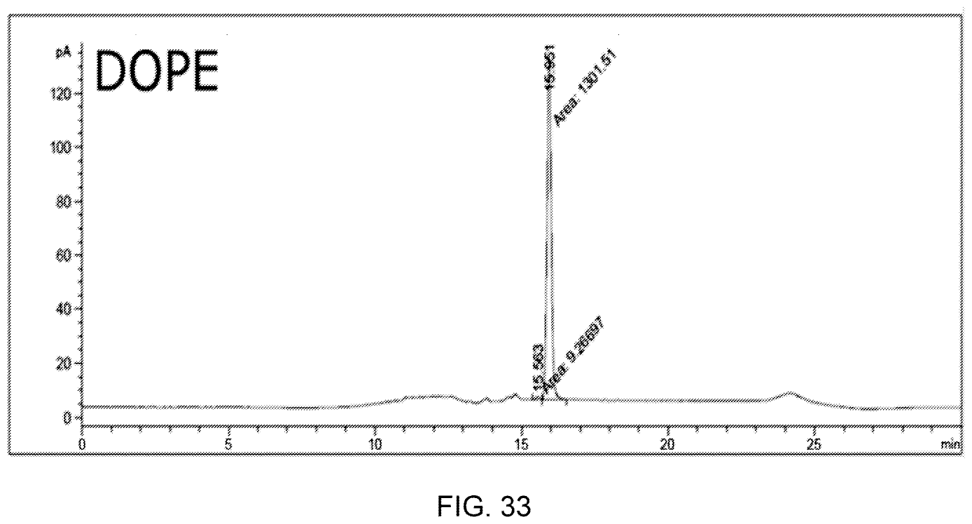

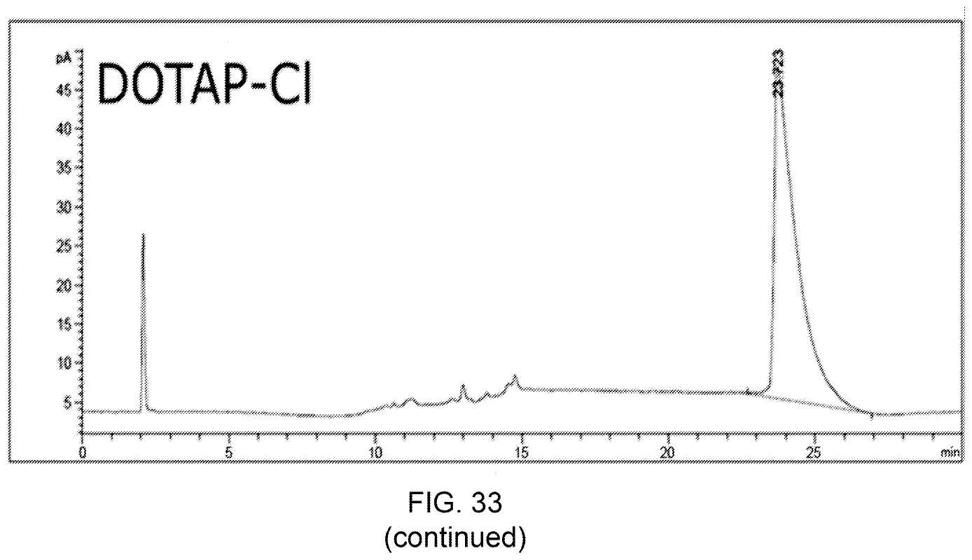

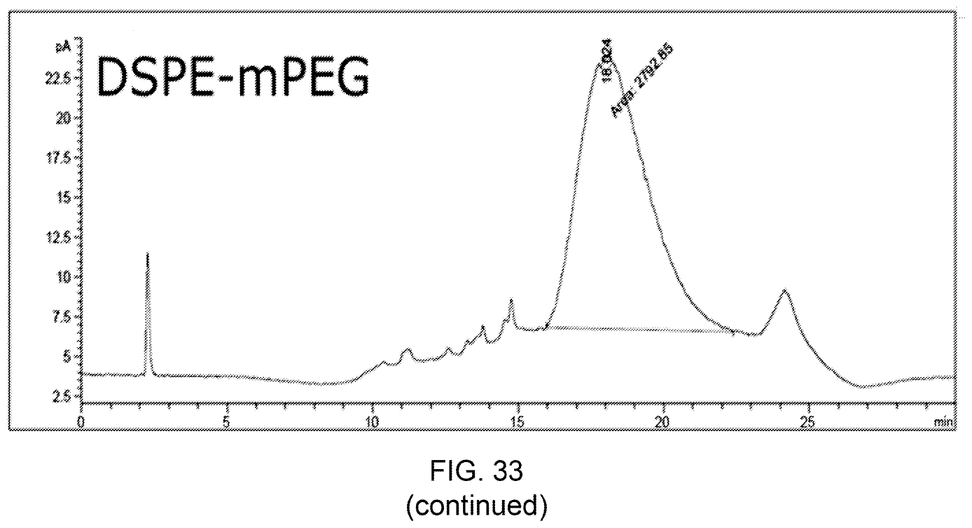

For the phenyl column, the lipid DOPE showed a peak eluting at 15.95 min. DSPE-mPEG2k showed a broad peak, mainly related to the polydispersity of the PEG chains, between 16 and 23 min. Finally, DOTAP-Cl eluted at approx. 24 min. For DOTAP-Cl we observed a slightly overlaps with a peak caused by the abrupt solvent switch at the end of the method. For this reason, the method initially used was optimized for earlier elution of lipids as reported in FIG. 33.

% Ammonium-acetate

| Time | Old gradient | New gradient |
|---|---|---|
| 00.0 min | 35% | 25% |
| 01.0 min | | 25% |
| 02.0 min | 35% | |
| 07.0 min | | 5% |
| 10.0 min | 5% | |
| 20.0 min | 5% | |
| 25.0 min | 35% | 5% |
| 25.1 min | | 25% |
| 30.0 min | 35% | 25% |

Finally, lipid mix were prepared and analyzed using the modified method at final product concentration of

| | |
|---|---|
| [DOTAP-Cl]_{fin} | 0.725 mg/ml |
| [DOPE]_{fin} | 0.73 mg/ml |
| [mPEG2000′DSPE]_{fin} | 0.145 mg/ml |

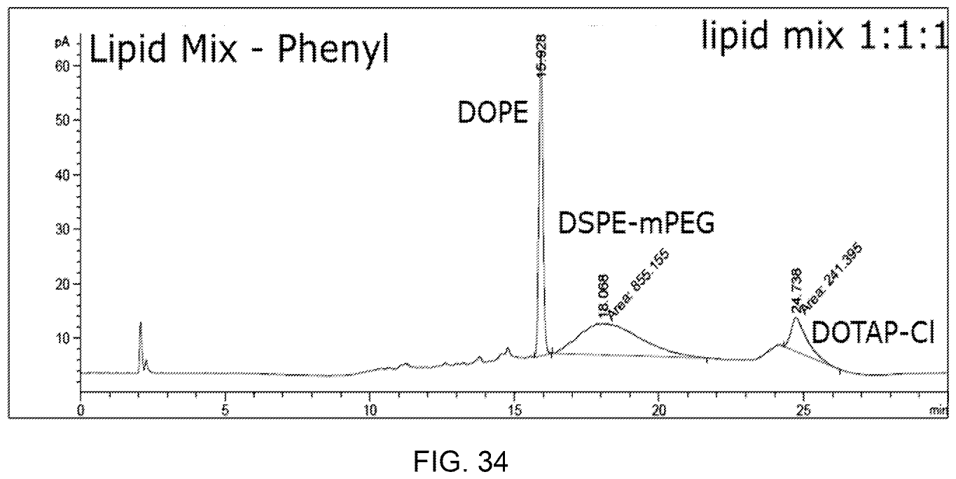

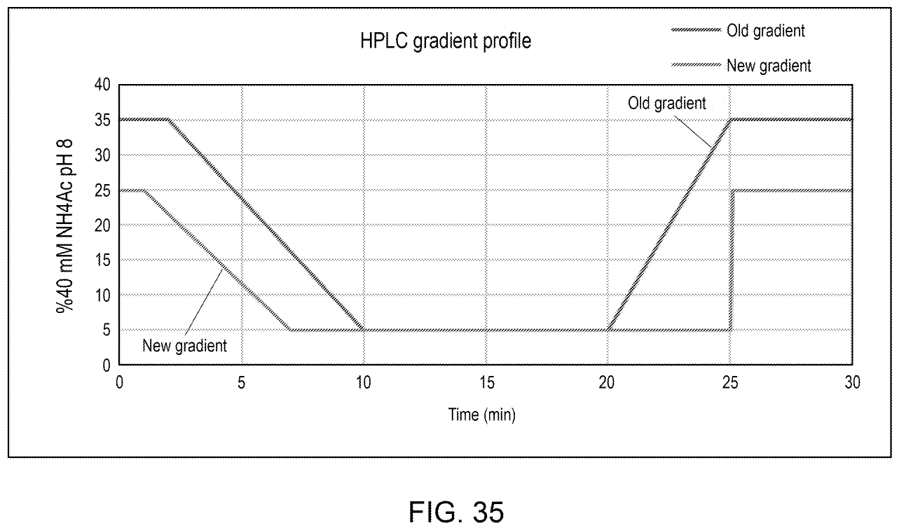

In FIG. 34 the chromatogram of the lipid mix is reported.

Experimental observations indicated good separation of the lipid peaks using the developed HPLC method. This method was therefore used to evaluate the lipid contents in the extruded solutions for all the samples.

The determination of the lipid content in the extruded sample is based on the preparation of a calibration curve for the three lipids followed by HPLC sample analysis using the method described above. As a representative experiment, the detailed procedure and the results obtained for experiment MVI0010 are reported here. A table containing the lipid recovery for all the analyzed samples is also reported at the end of the examples.

For lipid quantification in the extruded sample, standard lipids solutions were prepared using the prepared lipid stock solution as starting material. The standards used to construct the calibration curve are reported in Table 14.

TABLE 14
Standard solution used for lipid quantification

| | DOTAP-C1 | DOPE | DSPE-mPEG2000 |
|---|---|---|---|
| Std | (mg/ml) | (mg/ml) | (mg/ml) |
| 1 | 0.1 | 0.1 | 0.01 |
| 2 | 0.2 | 0.2 | 0.05 |
| 3 | 0.5 | 0.5 | 0.15 |
| 4 | 0.7 | 0.7 | 0.2 |
| 5 | 0.9 | 0.9 | 0.3 |

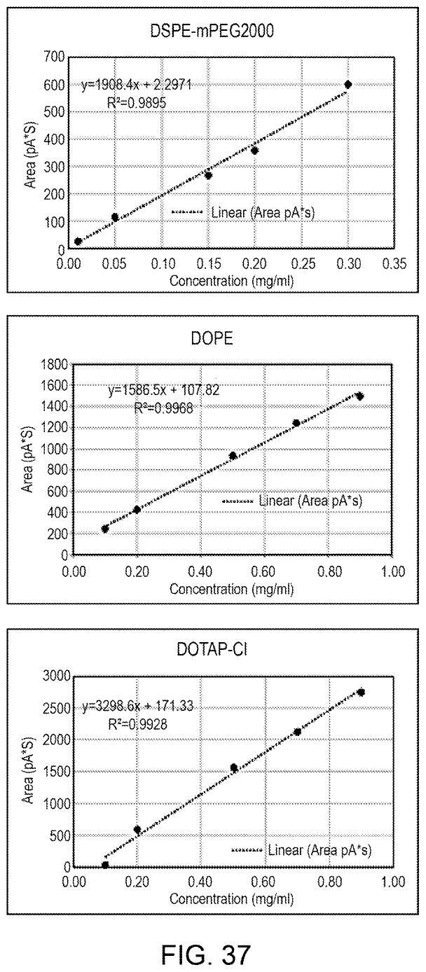

The chromatogram of each standard solution was determined and analyzed, and the calibration curve was constructed by reporting the area of the peaks vs. lipid concentration, for each lipid. FIG. 37 shows the calibration curve for each lipid obtained for sample MVI0010 using the standards reported in Table 14.

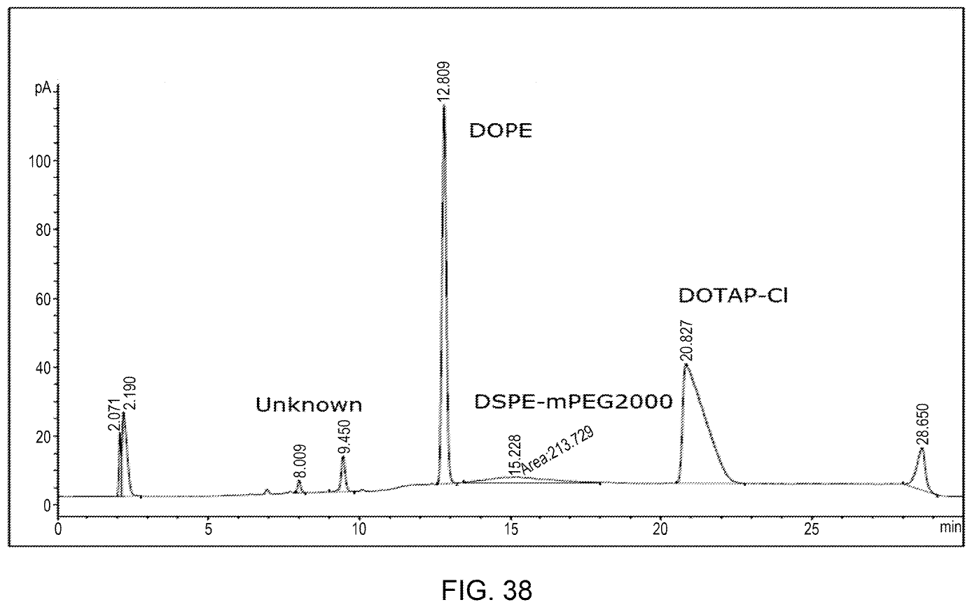

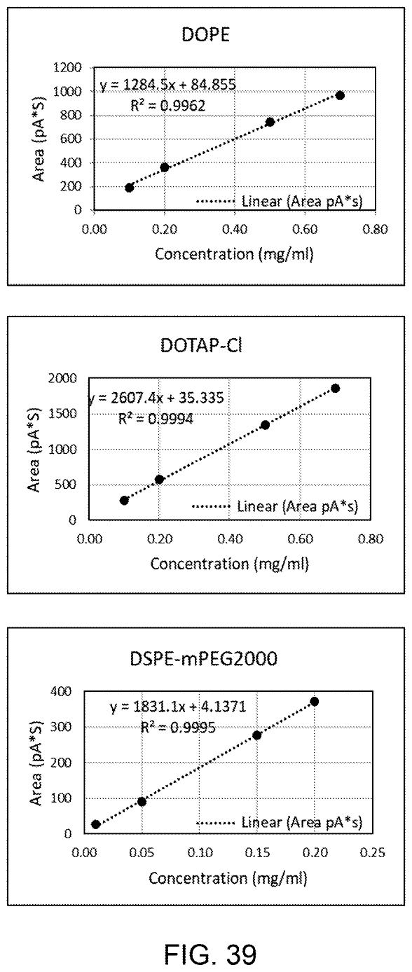

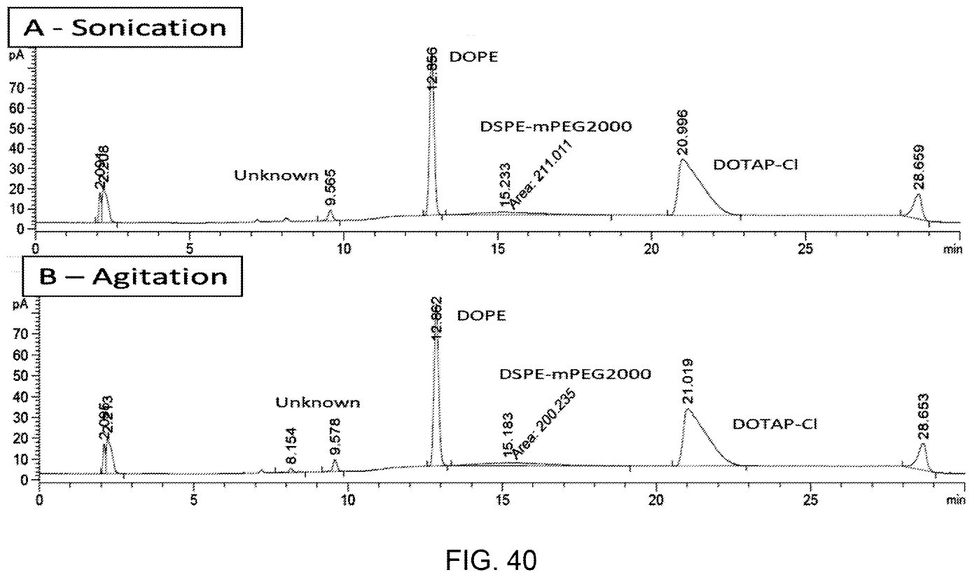

The calibration curves of the three lipids were used to determine the lipid recovery after extrusion. Sample 10 was generated following general experimental procedure of MeOH exposure of SiNP for activation and injection of lipid into the SiNPs solution (see FIG. 17). The solution was then extruded as previously described through 0.8 μm, 0.4 μm and 0.1 μm membranes at 60° C. The sample was next analyzed by HPLC, and the chromatogram is reported in FIG. 38.

The area of each peak was used to determine the concentration of the lipids in the sample post extrusion. Table 15 shows the recovery of lipids post-extrusion for experiment MVI0010.

TABLE 15
recovery of lipids post-extrusion for experiment MVI0010

| | Expected | | Recovery over |
|---|---|---|---|
| | concentration | Concentration | the expected |
| Component | (mg/ml) | (mg/ml) | concentration |
| DOPE | 0.730 | 0.628 | 86% |
| DSPE-mPEG2000 | 0.145 | 0.111 | 76% |
| DOTAP-C1 | 0.725 | 0.586 | 81% |

Finally, Table 16 summarizes the HPLC content for all the analyzed samples after extrusion using the described procedure

TABLE 16
HPLC content of the analyzed samples

| | | | | LIPID CONTENT | | | | | |
|---|---|---|---|---|---|---|---|---|---|
| | | | | | | DSPE- | | | |
| | | | | DOPE | | mPEG2000 | | DOTAP-Cl | |
| | | | | Conc. | | Conc. | | Conc. | |
| EXPERIMENT | | | | mg/ml | Recovery | mg/ml | Recovery | mg/ml | Recovery |
| SiSaf ref. Values | | | | 0.725 | 100% | 0.145 | 100% | 0.730 | 100% |
| MVI0008 | non-prehydrated + | 50 | ml | 0.495 | 68% | 0.113 | 78% | 0.408 | 56% |
| | injection | | | | | | | | |
| MVI0009 | non-prehydrated + | 50 | ml | 0.580 | 80% | 0.120 | 82% | 0.510 | 70% |
| | injection | | | | | | | | |
| MVI0010 | non-prehydrated + | 100 | ml | 0.628 | 86% | 0.111 | 76% | 0.586 | 81% |
| | injection | | | | | | | | |
| MVI0011 | non-prehydrated + | 100 | ml | 0.554 | 76% | 0.103 | 71% | 0.503 | 69% |
| | injection | | | | | | | | |
| MVI0012 | non-prehydrated + | 1 | L | 0.625 | 86% | 0.110 | 76% | 0.553 | 76% |
| | injection | | | | | | | | |

Experimental results indicated an average recovery post extrusion of:
DOPE: 79.2%
DSPE-mPE: 76.6%
DOTAP-CI: 70.4%

### Example 10—Stability Over Time

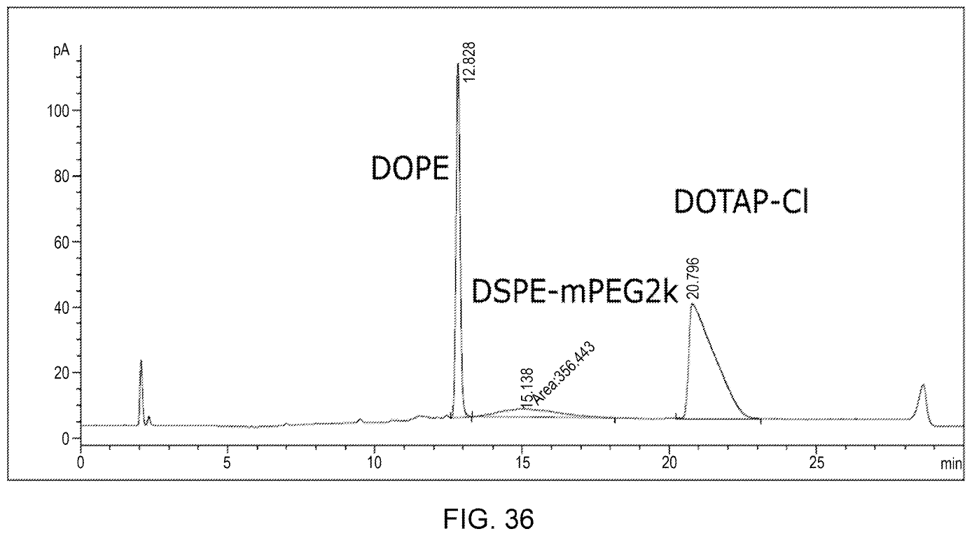

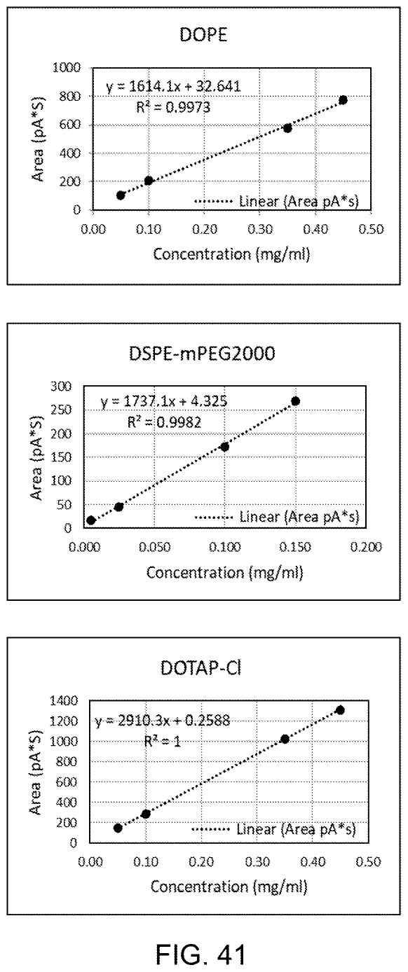

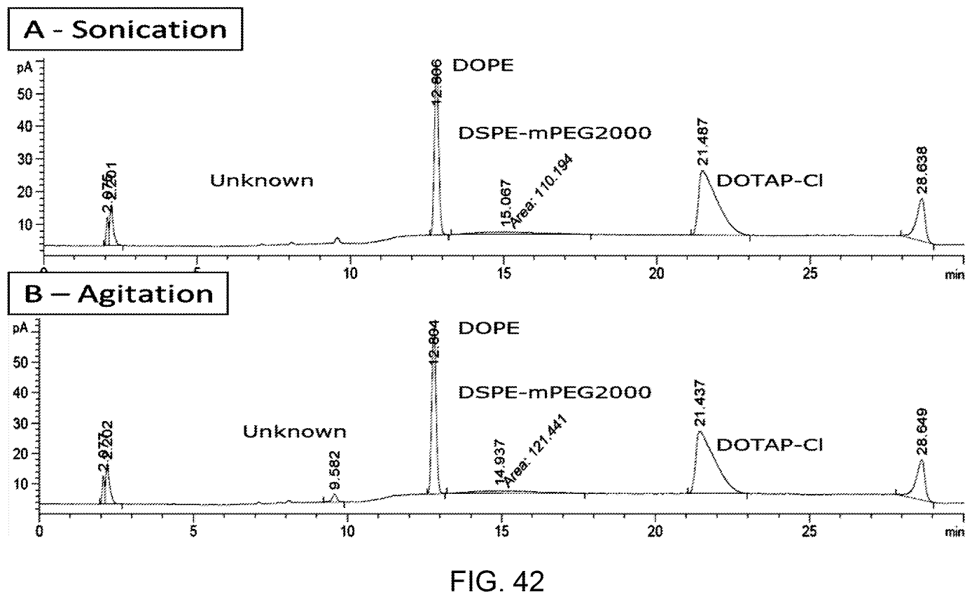

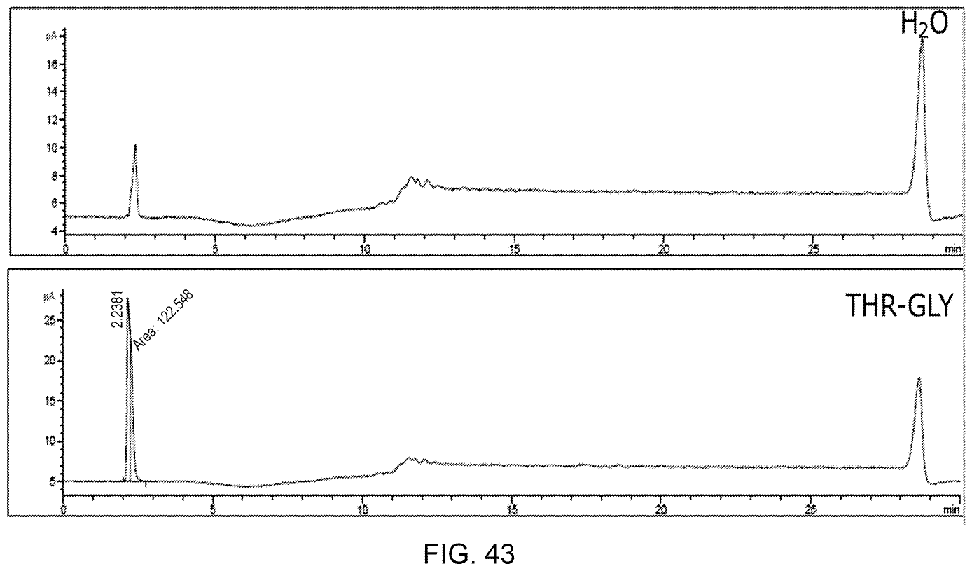

In the chromatograms of the samples post extrusion we noted the presence of unknown peaks at 7 minutes, 8 minutes and 9.5 minutes (see FIG. 36 for representative example). In order to investigate potential source of contamination, firstly the sample chromatograms of nuclease free water and THR-GLY solutions were analyzed, both reported in FIG. 41.

As indicated in FIG. 41, GLY and THR eluted directly at injection. Consequently, the peaks at 7 minutes, 8 minutes and 9.5 minutes do not seem to be related to GLY and THR.

To evaluate the evolution of the unknown peaks, HPLC and DLS features of the samples were analyzed over time. Samples were stored at room temperature (RT) and 4° C. and HPLC content and DLS features were measured at time 0 (as produced), after 1 week and after 2 weeks.

### (i) Investigation of the Samples Stored at Room Temperature

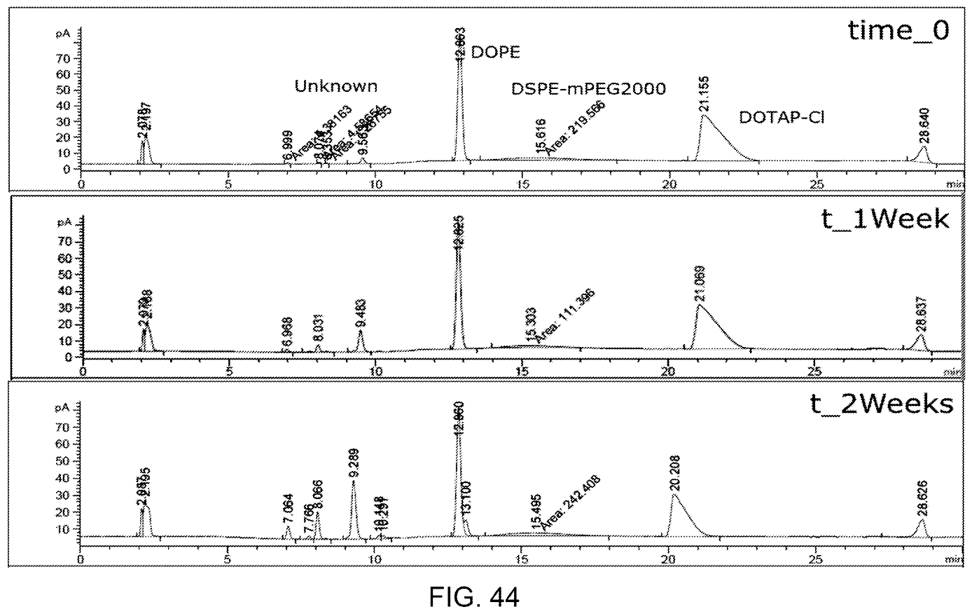

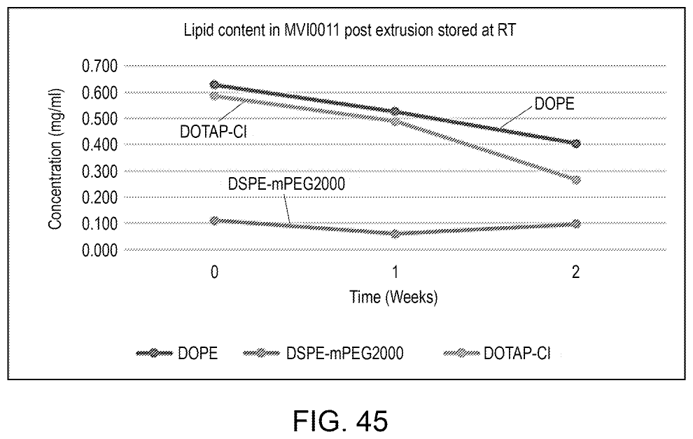

The evolution of the lipid content in samples stored at RT was determined at different time point, specifically at time 0, after 1 week and after 2 weeks. To do that, a calibration curve was constructed for the different time point. The variation of the lipid concentration was then determined by comparing the lipid concentration measured at time 0 and the lipid concentration measured at the different time points, for each lipid. In FIG. 44 the chromatograms for sample stored at RT up to 2 weeks are reported.

To further investigate the stability of the samples over time, DLS features were measured, and the results are reported in Table 17.

TABLE 17
DLS features of the analyzed sample at RT over time

| Week | Z-Average (nm) | PDI | Z-Potential (mV) |
|---|---|---|---|
| 0 | 68.62 +/− 0.82 | 0.153 +/− 0.014 | 63.3 +/− 5.20 |
| 1 | 71.59 +/− 0.96 | 0.158 +/− 00.17 | 60.3 +/− 1.02 |
| 2 | 72.02 +/− 0.26 | 0.143 +/− 0.875 | 65.1 +/− 0.86 |

Table 17 shows that the DLS features for samples stored at RT are largely maintained over time.

### (II) Investigation of the Samples Stored at 4° C.

The evolution of the lipid content was determined at different time for sample stored at 4° C. using same procedure indicated for samples stored at RT. Therefore, a calibration curve was constructed for each time point and lipid concentration determined by comparison between the concentration measured at time 0 and over time. In FIG. 44 the chromatograms at different time point for the analyzed sample at 4° C. are reported.

DLS and Zeta potential features were measured over time and the results are reported in Table 18.

TABLE 18
features of the analyzed sample at RT over time

| Week | Z-Average (nm) | PDI | Z-Potential (mV) |
|---|---|---|---|
| 0 | 68.62 +/− 0.82 | 0.153 +/− 0.014 | 63.3 +/− 5.20 |
| 1 | 69.51 +/− 0.83 | 0.168 +/− 0.015 | 66.4 +/− 2.46 |
| 2 | 73.32 +/− 1.49 | 0.148 +/− 0.008 | 62.6 +/− 0.86 |

As reported in Table 18, the DLS features are largely maintained over time also for samples stored at 4° C.

Experimental observations on the investigated samples suggested a decrease in concentration of lipids in samples stored at 4° C. and RT over time. This decrease can also be imputed to an instrumental variation because of the low measured concentration for each lipid: low peak area can lead to large experimental variation. Interestingly, increasing injection volume led to increased recovery of DSPE-mPEG lipid (114%). This observation also indicates an instrumental variation on the obtained results.

### Example 11—Assessment of Tangential Flow Flow Rate

Tangential flow filtration (TFF) is a rapid and efficient method for separation and purification of biomolecules. Within the context of this work, TFF is used to purify sample via diafiltration and eventually concentrate it to match the desired concentration range of the components. Through diafiltration, smaller molecules are washed through a membrane with defined pore size while larger molecules are retained in the retentate. For this reason, diafiltration was used to remove MeOH. In this work, we performed diafiltration by adding the diafiltration solution via a second pump to the sample feed reservoir at the same rate as filtrate is generated. In this way, the volume in the sample reservoir remains constant, but the MeOH freely permeate through the membrane and washed away. Due to their small molecular weight, THR and GLY can be removed during TFF. For this reason, we used a THR-GLY solution at same final concentration (0.1 mg/ml of THR and 0.05 mg/ml of GLY) as a diafiltration solution.

Adding a volume of diafiltration solution to the feed reservoir equal to the volume of product in the system, then concentrating back to the starting volume constitutes one diafiltration volume. Thus, for 100 mL of starting sample, 1 DV=100 mL. It has been previously observed that using 10 DV, the MeOH content is drastically reduced to values in the 100 ppm range values with continuous diafiltration (European standard for accepted MeOH content <3000 ppm).

For feasibility studies of TFF we used a membrane with the following characteristic:
Surface 0.02 m²
MWCO 100 kDa
Membrane HyStream. HyStream membrane is extremely hydrophilic in nature, resisting fouling from hydrophobic species such as, lipids. The membrane has good chemical resistance.
Architecture LP-screen. This type of channel is best suited for clarified feed streams over a wide range of viscosities.

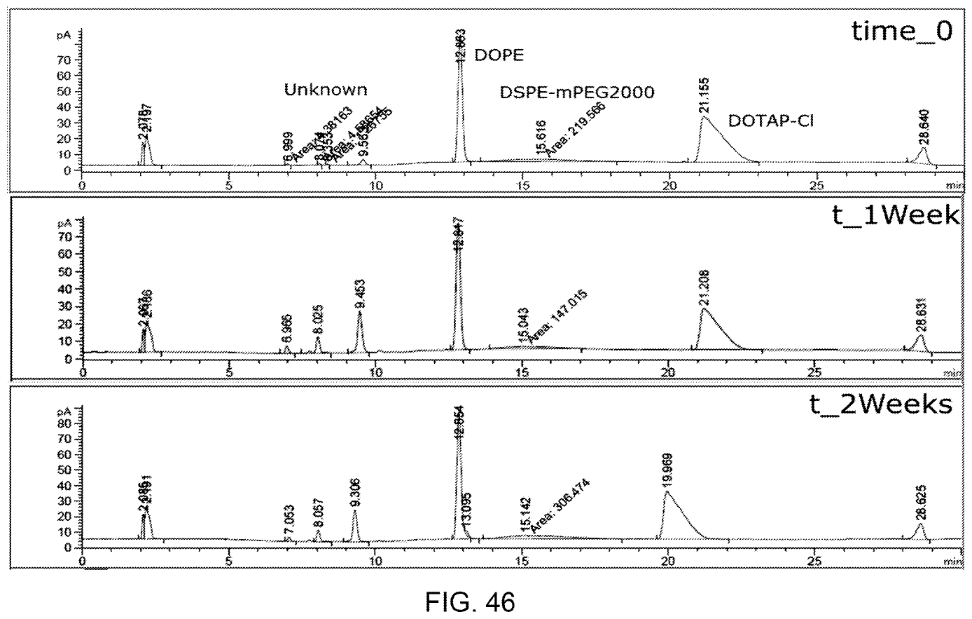

The general TFF setup used in this work is reported in FIG. 46.

The samples were generated by following the approach of NPs exposure to MeOH for activation and subsequent dilution in a THR-GLY aqueous solution. After that, mixed lipid solution was properly prepared and injected in the NP-THR-GLY dispersion. The solution was then firstly extruded through 0.8 μm, 0.4 μm and 0.1 μm membranes at 60° C. and subsequently purified by TFF using the experimental setup described in FIG. 46. To study the impact of the TFF on the system, DLS features, HPLC lipid content and MeOH content must be evaluated pre-TFF and post-TFF. DLS features and HPLC content analysis were performed as described in the previous sections. MeOH content investigation was performed by ¹H-NMR.

The general approach for determination of the MeOH content in the sample is based on the preparation of a calibration curve for MeOH. As a representative experiment, reported here are the detailed procedure and the results obtained for experiment MVI0010. At the end of the examples a table containing the determination of MeOH content for all the analyzed samples, is reported.

MeOH standard solutions with final volume of 1 ml were prepared at different MeOH concentration (Table 21).

TABLE 19
Concentration data used for construction
of the calibration curve for MeOH

| | [MeOH] | [MeOH] |
|---|---|---|
| Std | (ppm by volume) | Volume % |
| 1 | 50 | 0.005% |
| 2 | 100 | 0.01% |
| 3 | 300 | 0.03% |
| 4 | 500 | 0.05% |

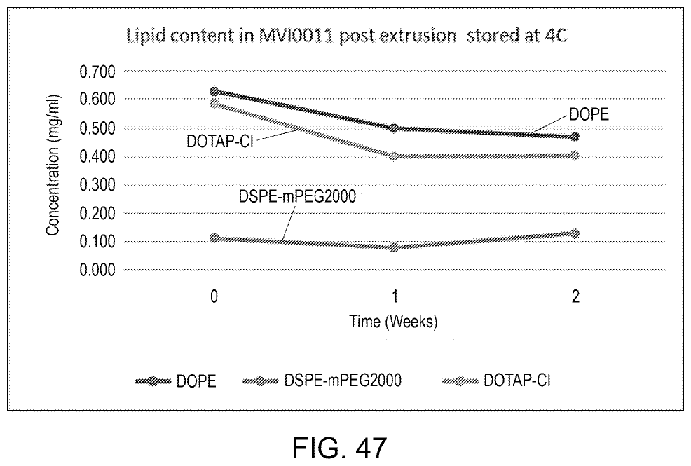

10% in volume of D₂O was added to the standards (100 μL D₂O). 600 μL of each standard were loaded in the NMR cuvette and all the standards were analyzed using ¹H-NMR. The calibration curve reported in FIG. 47. was constructed by reporting the area of the peaks related to MeOH signal at different MeOH concentration.

After preparing the standard curve, samples were analyzed to determine MeOH content. Pre TFF sample was prepared by proper dilution of the original sample to match the range of the calibration curve. For Sample 11, 1000× dilution was employed since the expected methanol content in the sample is about the 30% of the sample volume (see Table 2 as an example). Post TFF sample was analyzed concentrated. The final volume for each sample was 1000 μL. 10% in volume of D₂O has been therefore added to the samples (100 μL D₂O). 600 μL of each sample were loaded in the NMR cuvette and analyzed using 1H-NMR. The area of MeOH peak in the NMR spectra was then used to determine the concentration of the MeOH in the sample pre TFF and post TFF.

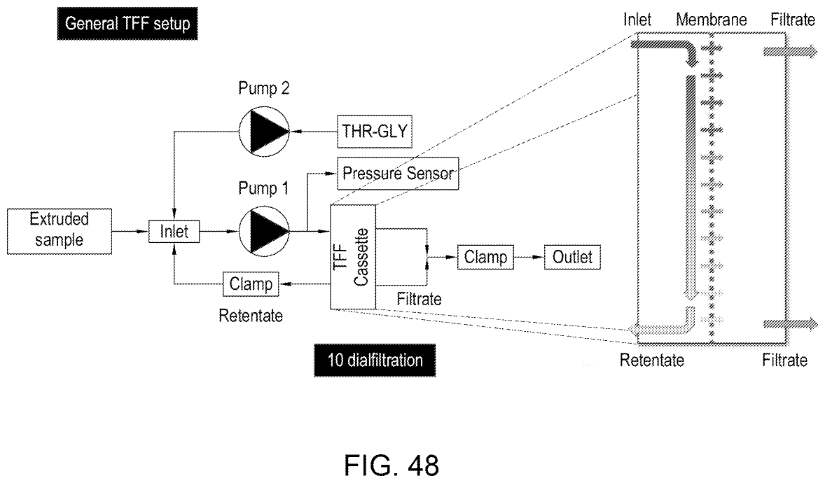

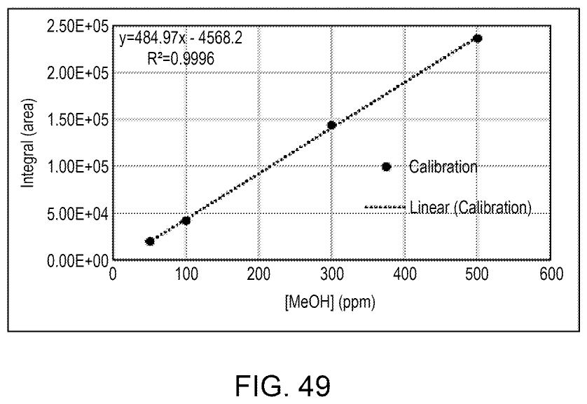

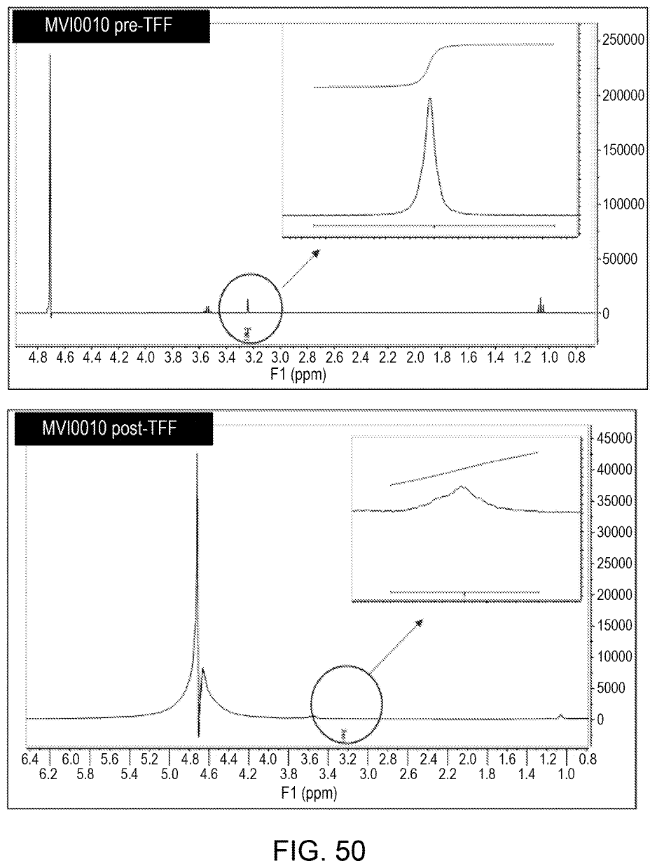

FIG. 48 shows the NMR signal for Sample 10 pre TFF and post TFF as representative result.

Table 20 reports the MeOH quantification in Sample 10 pre TFF and post TFF.

TABLE 20
MeOH quantification in sample MVI0010 pre and post-TFF

| | |
|---|---|
| Sample | [MeOH] |

| MVI0010 | ppm by volume | ppm by weight* |
|---|---|---|
| pre-TFF | 2.46 × 10⁵ | 2.43 × 10⁵ |
| post-TFF | 455.1 | 450.3 |

The estimation of MeOH content was performed considering the density of the MeOH—H₂O mixture 0.988 kg/L [2].

Using similar procedure described for MeOH determination in Sample 10, we also determined the MeOH content for the other analyzed samples, namely Sample 08 and Sample 09. In Table 21 the MeOH content pre and post TFF is reported for all the analyzed samples.

TABLE 21
MeOH quantification in the ananlyzed sample pre- and post-TFF

| | | | | MeOH Content | |
|---|---|---|---|---|---|
| EXPERIMENT | | | Vol | ppm by volume | ppm by weight |
| MVI0008 | | non-prehydrated + | 50 ml | 2.22 × 10⁵ | 2.19 × 10⁵ |
| | | injection | | | |
| | Pre-TFF | non-prehydrated + | 50 ml | 20.7 × 10⁵ | 2.05 × 10⁵ |
| | | injection | | | |
| MVI0009 | Post-TFF | non-prehydrated + | 50 ml | 209.29 | 207.2 |
| | | Injection | | | |
| | Pre-TFF | non-prehydrated + | 100 ml | 2.46 × 10⁵ | 2.43 × 10⁵ |
| | | Injection | | | |
| MVI0010 | Post-TFF | non-prehydrated + | 50 ml | 455.1 | 450.3 |
| | | Injection | | | |

Experimental observations indicate that the MeOH concentration decrease by three orders of magnitude as a results of TFF from 10⁵ to 10² for all the analyzed sample.

As already mentioned, HPLC and DLS features of the samples must be evaluated post-TFF to determine the feasibility of this process. These data are reported in Table 35 and Table 36 for all the analyzed samples pre TFF and post TFF.

TABLE 35
DLS features of the analyzed samples pre and post TFF

| | | | | Z- | | Z- |
|---|---|---|---|---|---|---|
| | | | | AVERAGE | | POTENTIAL |
| EXPERIMENT | VOL | | | (NM) | PDI | (MV) |
| MVI0009 | Pre-TFF | non- | 50 ml | 69.97 +/− 4.2 | 0.153 +/− 0.004 | 52.7 +/− 2.06 |
| | | prehydrated + | | | | |
| | | injection | | | | |
| | Post-TFF | non- | 50 ml | 79.06 +/− 0.4 | 0.173 +/− 0.026 | 38.6 +/− 1.88 |
| | | prehydrated + | | | | |
| | | injection | | | | |
| MVI0010 | Pre-TFF | non- | 100 ml | 84.64 +/− 1.64 | 0.221 +/− 0.015 | 48.3 +/− 1.31 |
| | | prehydrated + | | | | |
| | | injection | | | | |
| | Post-TFF | non- | 50 ml | 76.59 +/− 1.13 | 0.177 +/− 0.005 | 50.0 +/− 0.75 |
| | | prehydrated + | | | | |
| | | injection | | | | |

TABLE 24
HPLC content of the samples pre and post TFF

| | | | | LIPID CONTENT | | | | | |
|---|---|---|---|---|---|---|---|---|---|
| | | | | | | DSPE- | | | |
| | | | | DOPE | | mPEG2000 | | DOTAP-Cl | |
| EXPERIMENT | | | | Conc. | | Conc. | | Conc. | |
| VOL | | | | mg/ml | Recovery | mg/ml | Recovery | mg/ml | Recovery |
| MVI0009 | Pre- | non- | 50 ml | 0.580 | 80% | 0.120 | 82% | 0.510 | 70% |
| | TFF | prehydrated + | | | | | | | |
| | Post- | injection | 50 ml | 0.502 | 69% | 0.104 | 72% | 0.433 | 60% |
| | TFF | | | | | | | | |
| MVI0010 | Pre- | non- | 100 ml | 0.628 | 86% | 0.111 | 76% | 0.586 | 81% |
| | TFF | prehydrated + | | | | | | | |
| | Post- | injection | 50 ml | 1.242 | 85% | 0.212 | 73% | 1.134 | 78% |
| | TFF | | | | | | | | |

Experimental observations indicate that the DLS features are largely maintained post-TFF. Finally, the lipid recovery is also maintained after TFF.

Note that the sample MVI0010 was 2× concentrated through TFF. This was necessary to increase the Silicon content to match the desired silicon concentration in the final product.

### Example 12—Further Optimization and Reduction in Silicon

In this example, further optimization of the process for the development of micellar lipid particle is reported. We investigated three different experimental setups which are listed below:
i. preparation of micellar lipid particle batch filtering the SiNPs solution after activation.
ii. preparation of micellar lipid particle batch using ⅕ of the amount of Si normally used (4 mg).
iii. preparation of micellar lipid particle batch without the presence of DSPE-mPEG2000.
   Preparation of Micellar Lipid Particle Batch Filtering the SiNPs Solution after Activation (MVI0013)

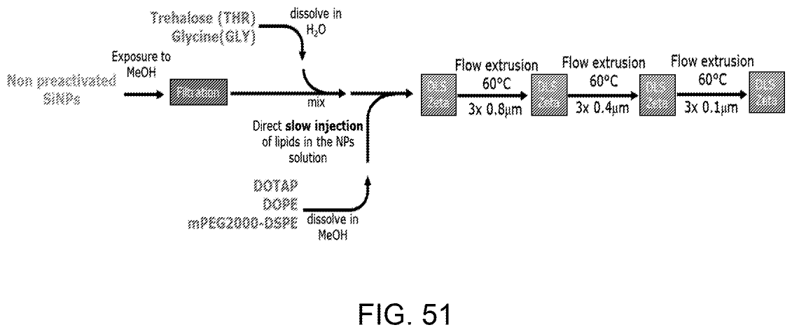

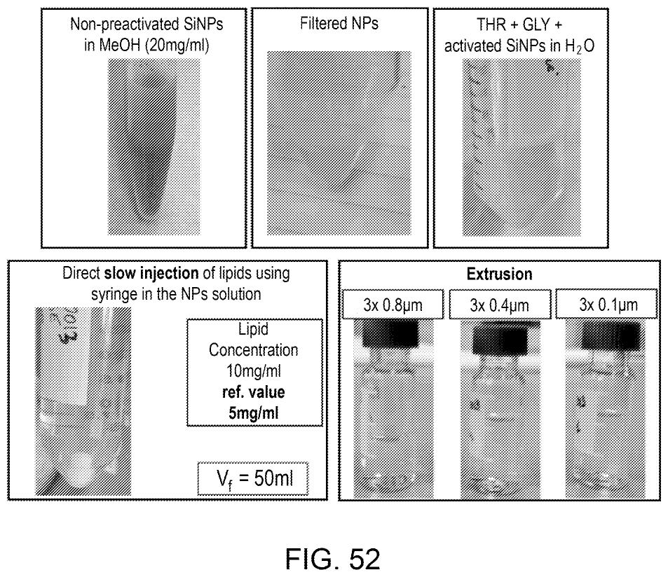

The experimental procedure for the preparation of micellar lipid particle batch filtering the SiNPs solution after activation is reported in FIG. 51. This experiment was named MVI0013.

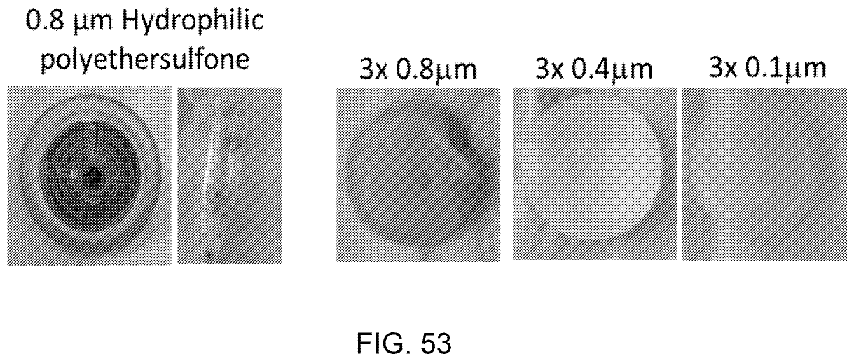

The micellar lipid particles were prepared as reported in FIG. 52 at final volume of 50 ml. The SiNP solution was manually filtered after MeOH activation using 0.8 μm hydrophilic polyethersulfone syringe filter. In flow extrusion through 25 mm extrusion membranes with pore size of 0.8 μm, 0.4 μm and 0.1 μm was performed at 60° C. The visual investigation of the solution obtained at each step for experiment MVI0007 is reported in FIG. 52. Pictures of the 0.8 μm hydrophilic polyethersulfone syringe filter and of the extrusion membranes after respectively filtration and extrusion steps are reported in FIG. 53.

In FIG. 52, the filtered solution appears yellowish, without the presence of large aggregates, already suggesting the removal of most of the aggregated SiNPs in the solution. FIG. 53 also confirms this observation as indicated by the presence of the brown layer of NPs on the 0.8 μm syringe filter shown in FIG. 53 A. Moreover, the 0.8 μm and 0.4 μm extrusion membranes are completely clean, as indicated by the absence of any brownish-colored area, whilst the 0.1 μm membrane still captured some of the smaller aggregates of SiNPs.

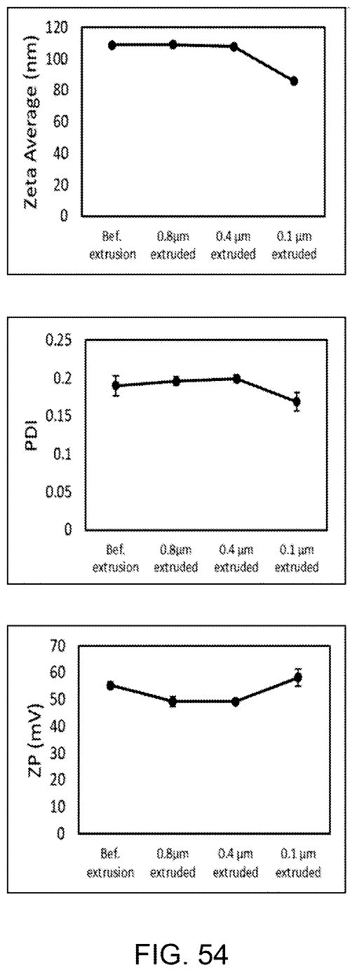

DLS and Zeta potential analysis of the MVI0013 sample were performed before extrusion and after each extrusion step. The obtained values are compared with SiSaf reference values. The results of the DLS investigation on the sample is reported in Table 37 and in FIG. 54.

TABLE 37
DLS and Zeta potential investigation of MVI0013
before extrusion and after each extrusion step.
The SiSaf reference values are also reported

| SAMPLE | Z-Average | | Zeta Potential |
|---|---|---|---|
| MVI0007 | (nm) | PDI | (mV) |
| SiSaf ref. value | 108 +/− 0.50 | 0.089 +/− 0.01 | 39.3 +/− 0.95 |
| Before extrusion | 108.9 +/− 1.48 | 0.190 +/− 0.013 | 55.3 +/− 1.26 |
| 0.8 μm extruded | 109.1 +/− 0.91 | 0.196 +/− 0.006 | 49.2 +/− 1.74 |
| 0.4 μm extruded | 107.7 +/− 0.81 | 0.199 +/− 0.005 | 49.3 +/− 0.5 |
| 0.1 μm extruded | 85.81 +/− 1.027 | 0.169 +/− 0.012 | 58.2 +/− 3.08 |

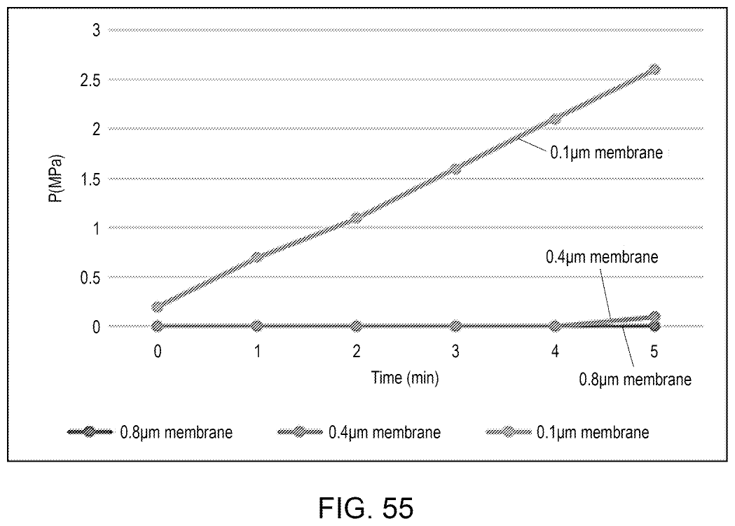

DLS investigation indicates that the 0.8 μm syringe filter is removing the majority of the aggregates of SiNPs, as indicated by the initial value of the Z-average (value pre-extrusion). The Z-average value is largely maintained up to the 0.4 μm extrusion step also confirming aggregates removal by the syringe filter. Extrusion pressure is also reported in FIG. 55 and resemble similar indication: the pressure on 0.8 μm and 0.4 μm membranes is almost zero, confirming the absence of large aggregates in the extruded solution, whilst on membrane 0.1 μm pore size some pressure has been reported. Finally, values of PDI and Zeta potential are largely maintained aver all the steps.

TABLE 38
HPLC content investigation on MVI0013

| | EXPECTED | | RECOVERY |
|---|---|---|---|
| | CONCEN- | CONCEN- | OVER THE |
| | TRATION | TRATION | EXP. CONCEN- |
| COMPONENT | (MG/ML) | (MG/ML) | TRATION |
| DOPE | 0.730 | 0.717 | 98% |
| DSPE- | 0.145 | 0.130 | 90% |
| mPEG2000 | | | |
| DOTAP-Cl | 0.725 | 0.700 | 96% |

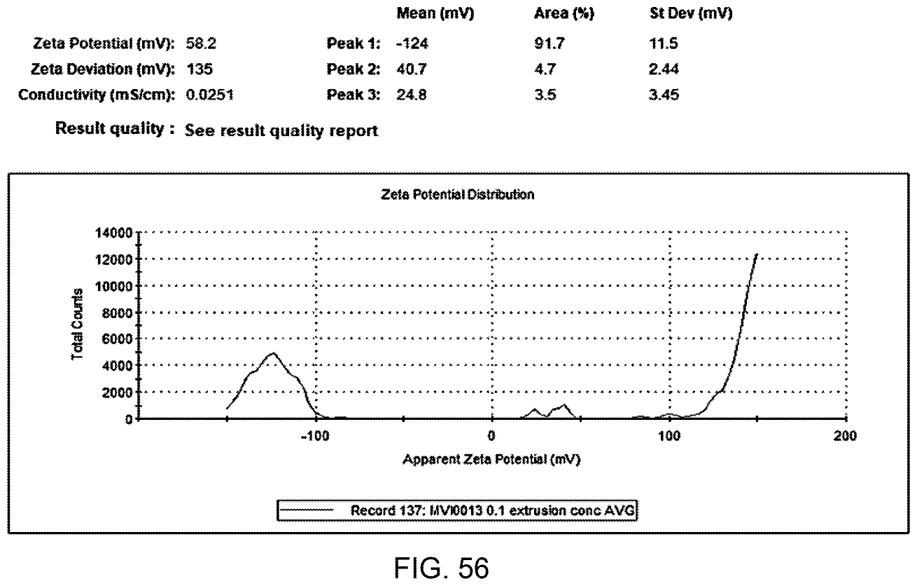

Experimental observation in experiment MVI0013 indicate that DLS features in line with expected values. However, we noted the absence of a well-defined peak in the Zeta Potential analysis (FIG. 56). Finally, HPLC quantification shows 90% of lipid recovery.

### (ii) Preparation of Micellar Lipid Particle Batch Using ⅕ of the Amount of Si (MVI0014).

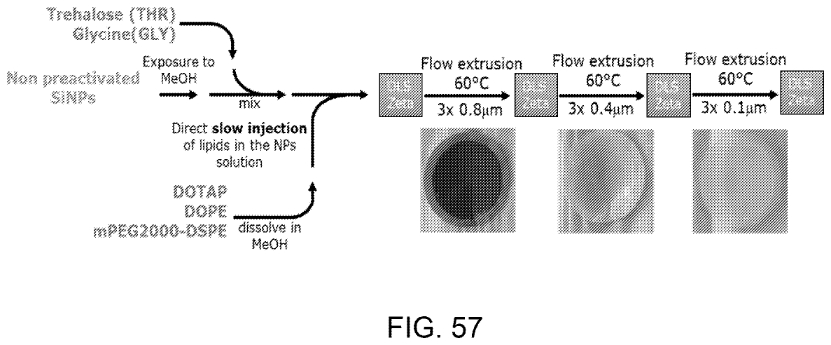

The experimental procedure for preparation of the micellar lipid particle batch filtering the SiNPs solution after activation is reported in FIG. 57. This experiment was named MVI0014.

The micellar lipid particles were prepared as reported in FIG. 57 at final volume of 50 ml. The SiNP solution was generated by reducing the amount of silicon normally used in all the other experiments as indicated in Table 39.

TABLE 39
Theoretical final concentration of each component for preparation
of the MVI0014 micellar lipid particles batch.

| | | |
|---|---|---|
| [SiNPs]_{fin} | 0.02 | mg/ml |
| [THR]_{fin} | 0.1 | mg/ml |
| [GLY]_{fin} | 0.05 | mg/ml |
| [DOTAP-Cl]_{fin} | 0.725 | mg/ml |
| [DOPE]_{fin} | 0.73 | mg/ml |
| [mPEG2000′DSPE]_{fin} | 0.145 | mg/ml |

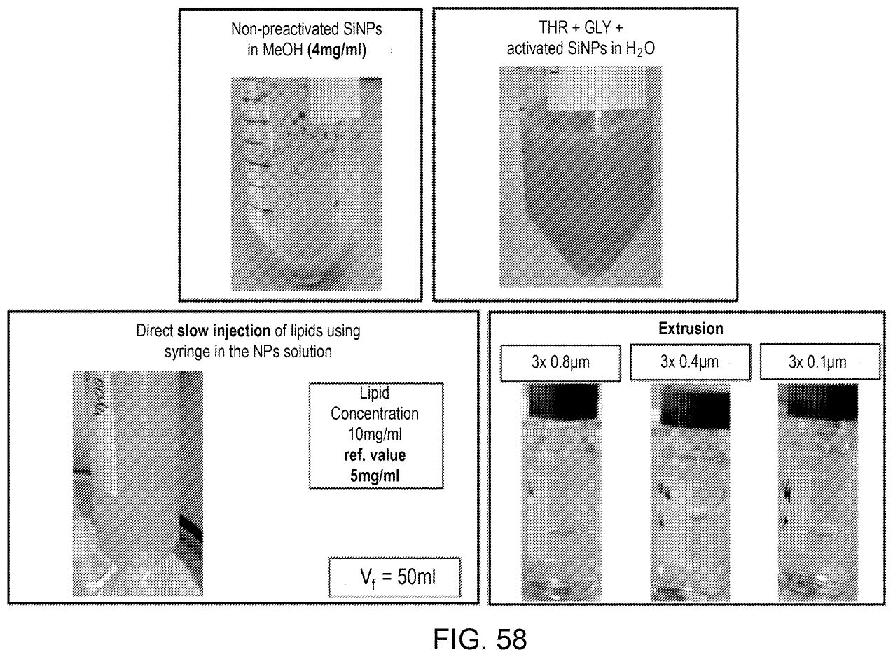

To do that, the concentration of the SiNPs solution was reduced to 4 mg/ml, by weighing 4 mg of non-activated SiNPs and exposing them to 1 ml of MeOH. Visual investigation of the obtained solution is reported in FIG. 58.

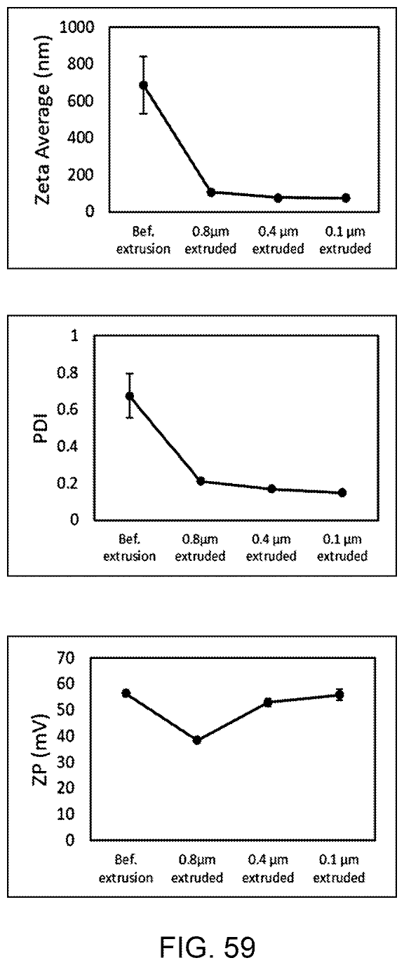

DLS and Zeta potential analysis of the MVI0014 sample was performed before extrusion and after each extrusion step. The obtained values were compared with SiSaf reference values. The results of the DLS investigation on the sample is reported in Table 40 and in FIG. 59.

TABLE 40
DLS and Zeta potential investigation of MVI0014 before extrusion and after
each extrusion step. The SiSaf reference values are also reported

| SAMPLE | Z-Average | | Zeta Potential |
|---|---|---|---|
| MVI0014 | (nm) | PDI | (mV) |
| SiSaf ref. value | 108 +/− 0.50 | 0.089 +/− 0.01 | 39.3 +/− 0.95 |
| Before extrusion | 685.7 +/− 155 | 0.675 +/− 0.12 | 56.4 +/− 0.229 |
| 0.8 μm extruded | 107.7 +/− 2.066 | 0.211 +/− 0.009 | 38.4 +/− 0.352 |
| 0.4 μm extruded | 75.99 +/− 0.95 | 0.169 +/− 0.002 | 52.9 +/− 1.5 |
| 0.1 μm extruded | 74.95 +/− 0.644 | 0.148 +/− 0.009 | 55.9 +/− 2.17 |

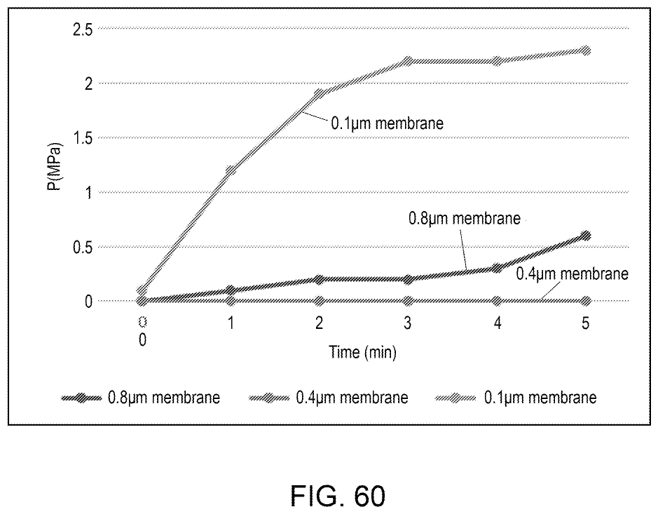

FIG. 60 reports the investigation of the extrusion pressure on MVI0013. As indicated in FIG. 60, values of extrusion pressure were relatively low, and this could be related to the lower amount of silicon used in this experiment.

Finally, HPLC content was investigated, and the results are reported in Table 41.

TABLE 41
HPLC content investigation on MVI0014

| | EXPECTED | | RECOVERY |
|---|---|---|---|
| | CONCEN- | CONCEN- | OVER THE |
| | TRATION | TRATION | EXP. CONCEN- |
| COMPONENT | (MG/ML) | (MG/ML) | TRATION |
| DOPE | 0.730 | 0.614 | 84% |
| DSPE- | 0.145 | 0.118 | 82% |
| mPEG2000 | | | |
| DOTAP-Cl | 0.725 | 0.579 | 80% |

Experimental observation in experiment MVI0014 indicated that DLS features are in line with expected values. HPLC quantification shows 80% of lipid recovery.

(iii) Preparation of Micellar Lipid Particle Batch without the Presence of DSPE-mPEG2000 (MVI0015)

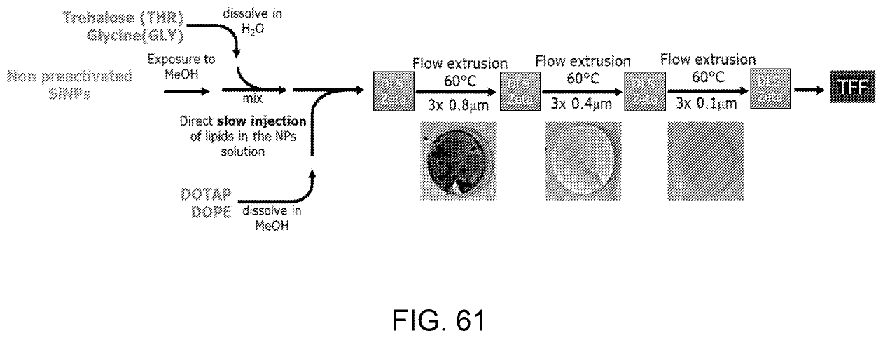

The experimental procedure for preparation of micellar lipid particle batch filtering the SiNPs solution after activation is reported in FIG. 61. This experiment was named MVI0015. Note that after extrusion, TFF was performed to remove MeOH and concentrate the sample by a factor of two.

The micellar lipid particles were prepared as reported in FIG. 61 at final volume of 200 ml (pre-TFF). The concentration of the lipid in the final solution was modified as reported in Table 42.

TABLE 42
Theoretical final concentration of each component for preparation
of the MVI0015 micellar lipid particles batch.

| | | |
|---|---|---|
| [SiNPs]_{fin} | 0.1 | mg/ml |
| [THR]_{fin} | 0.1 | mg/ml |
| [GLY]_{fin} | 0.05 | mg/ml |
| [DOTAP-Cl]_{fin} | 0.775 | mg/ml |
| [DOPE]_{fin} | 0.825 | mg/ml |
| [mPEG2000′DSPE]_{fin} | 0 | mg/ml |

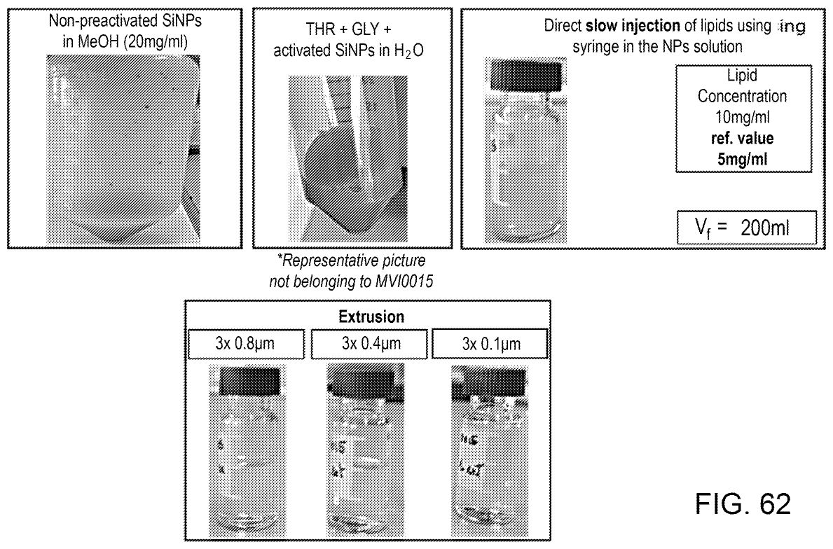

Visual investigation of the obtained solution is reported in FIG. 62.

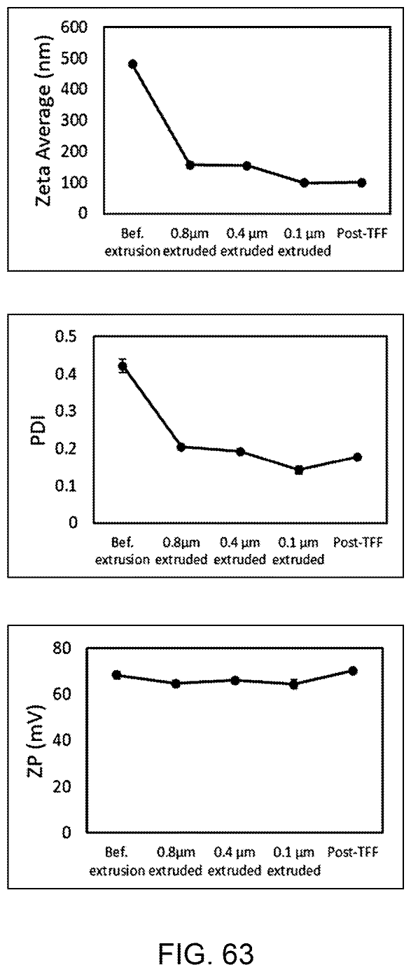

After extrusion, TFF was performed as indicated in section 2.6 Feasibility of the Tangential Flow Filtration and the MVI0015 sample purified and 2× concentrated. DLS and Zeta potential features were investigated, and results are reported in Table 43 and FIG. 63.

TABLE 43
DLS and Zeta potential investigation of MVI0015
before extrusion and after each extrusion step.
The SiSaf reference values are also reported

| SAMPLE | Z-Average | | Zeta Potential |
|---|---|---|---|
| MVI0014 | (nm) | PDI | (mV) |
| SiSaf ref. value | 108 +/− 0.50 | 0.089 +/− 0.01 | 39.3 +/− 0.95 |
| Before extrusion | 480.8 +/− 0.421 | 0.421 +/− 0.018 | 68.3 +/− 1.63 |
| 0.8 μm extruded | 157.7 +/− 2.043 | 0.205 +/− 0.003 | 64.6 +/− 1.54 |
| 0.4 μm extruded | 154.4 +/− 2.055 | 0.193 +/− 0.007 | 66.1 +/− 1.02 |
| 0.1 μm extruded | 99.41 +/− 1.04 | 0.142 +/− 0.011 | 64.4 +/− 2.01 |
| Post-TFF | 100.5 +/− 0.81 | 0.178 +/− 0.005 | 70.3 +/− 1.15 |

HPLC content was also investigated, and the results reported in Table 44.

TABLE 44
HPLC content pre TFF and post TFF for MVI0015

| | | CONCENTRATION | | RECOVERY OVER | |
|---|---|---|---|---|---|
| | | (MG/ML) | | THE EXPECTED | |
| | EXPECTED | Pre-TFF | Post-TFF | CONCENTRATION | |
| | CONC | (approx. | (approx. | | Post-TFF |
| COMPONENT | (MG/ML) | 180 ml) | 100 ml) | Pre-TFF | (2x dil) |
| DOPE | 0.825 | 0.778 | 1.062 | 94% | 64% |
| DOTAP-Cl | 0.775 | 0.703 | 0.916 | 91% | 59% |

Finally, the MeOH content was investigated by ¹H-NMR and the results are reported in Table 45.

TABLE 45
Methanol content in MVI0015

[MeOH]

| Sample | | ppm by volume | ppm by weight* |
|---|---|---|---|
| MVI00015 | pre-TFF | 7.21 × 10⁴ | 7.14 × 10⁴ |
| | post-TFF | 19.47 | 19.27 |

The MeOH content measured in MVI0015 post-TFF showed a value which fall below the lowest concentration point in the calibration curve, which was 50 ppm. This value may not be accurate. However, it indicated almost complete removal of MeOH from the sample.

Experimental observations in experiment MVI0015 (absence of DSPE-mPEG2000) indicate that the DLS features are in line with reference values and that the DLS features are largely maintained post-TFF. The HPLC quantification shows 90% of lipid recovery pre-TFF, however we observed a decrease in lipid recovery post-TFF (90% to 60%).

### Example 13—Determination of Silicon Content

The silicon content was determined by ICP-OES at SiSaf by investigating samples prepared with the different experimental approach described in previous sections. In detail, the silicon content in sample prepared using the original SiSaf experimental procedure via exposure of preactivated SiNPs to the lipid film (experiment MVI0004) was determined and compared to the silicon content measured using the modified experimental procedure of lipid injection into the non-preactivated SiNP solution (non-prehydrated SiNP+lipid injection). Table 46 reports a brief description of the analyzed samples for convenience.

TABLE 46
Brief description of the samples analyzed
for Silicon content determination

| | |
|---|---|
| MVI0004 | prehydrated + film hydration |
| MVI0005C | non-prehydrated + injection |
| MVI0009 | non-prehydrated + injection + TFF |
| MVI0010 | non-prehydrated + injection + TFF 2x conc. |
| MVI0011 | non-prehydrated + injection + 0.8 μm filtration of the |
| | SNiP-lipid solution pre-extrusion |
| MVI0013 | non-prehydrated + injection + 0.8 μm NP filtration after |
| | activation |
| MVI0014 | non-prehydrated + injection using ⅕ of the amount of Si |
| MVI0015 | non-prehydrated + injection without the presence of DSPE- |
| | mPEG2000 + TFF 2x conc. |
| MVI0022 | non-prehydrated + injection + TFF 2x conc. − 2 Kg |
| | batch size |

Table 47 summarizes the results of the ICP-OES investigation for all the analyzed samples.

TABLE 47
Investigation of the Silicon content using different
experimental approach for all the analyzed samples.

| | SILICON CONTENT |
|---|---|
| SAMPLE | (MG/L) |
| SiSaf ref. value | 2.10 |
| MilliQ water | 0.0076 |
| Nuclease free water | 0.102 |
| MVI0004 | 2 |
| MVI0005C | 1.2 |
| MVI0009 | 1.19 |
| MVI0010 | 2.18 |
| MVI0011 | 4.88 |
| MVI0013 | 3.1 |
| MVI0014 | 3.18 |
| MVI0015 | 3.33 |
| MVI0022 | 2.9 |

The reference value for silicon content in the final product is 2.1 mg/L. This value was also found in experiment MVI0004 replicating the original SiSaf procedure. On the other hand, using the experimental procedure reported in MVI0005C and MVI0009 (non-prehydrated SNiP+lipid injection), the silicon content was found to be 1.2 mg/L. By the 2× concentration approach via TFF applied to MVI0010 reported in Section 2.6 Feasibility of the Tangential Flow Filtration, we recovered the silicon content, which was found to be 2.18 mg/L in the sample post-TFF. Interestingly, a certain variability in the results have been observed in samples MVI0011 to MVI0015, where experimental procedure was modified as indicated in Table 46.

### Example 14—Manufacture of a 2 kg Batch

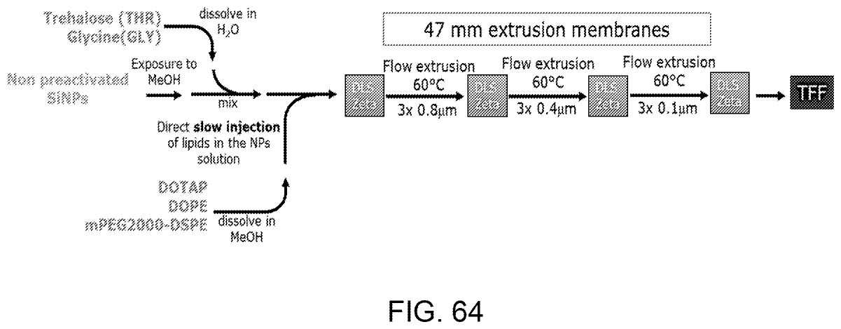

In this example, the results of the production of the 2 kg micellar lipid particles batch are reported. Experimental procedure for the generation of the 2 kg batch is depicted in FIG. 64. This experiment was named MVI0022.

The obtained batch was characterized by DLS and Zeta potential, HPLC to determine lipid content and ¹H-NMR for the MeOH quantification.

For the calculation, the density of the SiNPs—lipid dispersion was assumed as 1 g/cm³. Consequently, 4 L of the SNiP-lipid solution which was 2× concentrated by TFF. The final volume was 2 L, equal to 2 kg. Table 48 report the theoretical pre and post concentration for all the components. Note that concentration of THR and GLY is maintained post TFF since we used a THR-GLY solution at same final concentration (0.1 mg/ml of THR and 0.05 mg/ml of GLY) as a diafiltration solution.

TABLE 48
Theoretical pre-TFF and post-TFF concentrations

| Theoretical pre-TFF Solution | | | | Theoretical post-TFF Solution | | |
|---|---|---|---|---|---|---|
| Pre-TFF Volume | 4000 | ml | [symbol] | Post-TFF Volume | 2000 | ml |
| [SiNPs]_{fin} | 0.1 | mg/ml | | [SiNPs]_{fin} | 0.2 | mg/ml |
| [THR]_{fin} | 0.1 | mg/ml | | [THR]_{fin} | 0.1 | mg/ml |
| [GLY]_{fin} | 0.05 | mg/ml | | [GLY]_{fin} | 0.05 | mg/ml |
| [DOTAP-Cl]_{fin} | 0.725 | mg/ml | | [DOTAP-Cl]_{fin} | 1.45 | mg/ml |
| [DOPE]_{fin} | 0.73 | mg/ml | | [DOPE]_{fin} | 1.46 | mg/ml |
| [mPEG2000′DSPE]_{fin} | 0.145 | mg/ml | | [mPEG2000′DSPE]_{fin} | 0.29 | mg/ml |

For preparation of the 2 kg micellar lipid particles batch, aqueous SiNPs-THR-GLY solution was prepared using standard procedure of SiNP activation in MeOH (MeOH dispersion+sonication) followed by SiNP dilution in aqueous THR-GLY solution.

To do that, 400 mg of non-preactivated SiNP were weighted and transferred to a 50 ml sterile Falcon tube and 20 ml of MeOH was added for activation. THR-GLY aqueous solution was prepared by dissolving 400 mg of THR and 200 mg of GLY in 380 ml of nuclease free water (Nalgene sterile bottle). After SiNP activation 30 minutes at RT, the SiNPs were added to the THR-GLY solution and the obtained dispersion sonicated 60 min at 50° C. 10 mg/ml stock solution of DOTAP-Cl, DOPE and mPEG2000-DSPE were prepared by dissolving 3000 mg of DOTAP-Cl in 300 ml MeOH, 3000 mg of DOPE in 300 ml MeOH and 600 mg of mPEG2000-DSPE in 60 ml MeOH. Lipid solutions were next sonicated 30 minutes at 40° C. 400 ml of the SiNPs-THR-GLY solution were transferred to a 5 L sterile bottle. Lipids were slowly injected using Knauer K501 HPLC pump. The flow rate used for addition was 6 ml/min. After lipid addition, nuclease free water was added up to 4 L and the batch was left under stirring for homogenization. Table 49 reports the prepared stock solutions and volume used for the generation of the 4 L solution.

TABLE 49
Stock solutions and volume used for
the generation of the 4 L solution
Stock Solution

| | | |
|---|---|---|
| Volume of the SiNPs-THR-GLY H₂O solution | 400 | ml |
| [SiNPs] | 1 | mg/ml |
| [THR] | 1 | mg/ml |
| [GLY] | 0.5 | mg/ml |
| Volume of the Lipid stock solution (MeOH) | 300 | ml |
| [DOTAP-Cl]stock | 10 | mg/ml |
| [DOPE]stock | 10 | mg/ml |
| Volume of the PEG-Lipid stock solution (MeOH) | 60 | ml |
| [mPEG2000′DSPE]stock | 10 | mg/ml |

Volumes needed for generation of biocourier

| | | |
|---|---|---|
| SiNPs-THR-GLY solution | 400 | ml |
| DOTAP-Cl | 290 | ml |
| DOPE | 292 | ml |
| mPEG2000′DSPE | 58 | ml |
| Mixed lipid solution | 640 | ml |
| H₂O to add up | 2960 | ml |

| | |
|---|---|
| Final Volume | 4000 ml (15% |
| | in vol MeOH) |

The 4 L crude was prepared without any specific complication and stored overnight at 4° C. As usual, the presence of large aggregates of SiNPs was observed.

In flow extrusion over three-in-a-row 47 mm extrusion membranes with a pore size of 0.8 μm, 0.4 μm and 0.1 μm was then performed at 60° C. Knauer K501 pump with pressure sensor and 50 ml/min stainless steel pump head was used for extrusion. Flow rate was set to 50 ml/min

The visual investigation for the MVI0022 is reported in Table 50.

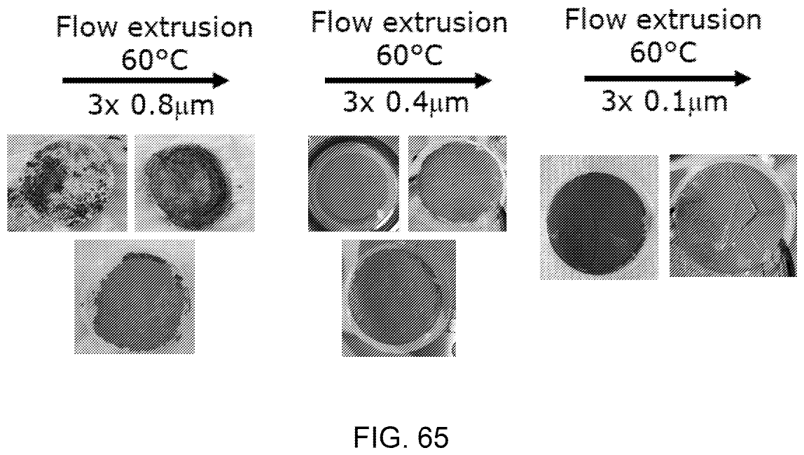

Notably, the SiNPs aggregates saturated the membranes as reported in FIG. 65. Extrusion membranes were changed during extrusion due to pressure fluctuations.

TFF was performed according to the MVI0010 setup. For MVI0022 we increased the surface area of the TFF cassette to 0.5 m² TFF membranes, maintaining the other features of the cassette used for extrusion of small size batches (HyStream, MWCO 100 kD, LP screen channel). Importantly, before TFF the system was depyrogenated by flushing the entire system with 0.2 M NaOH solution for 1 h. This approach was introduced at this stage for the production of the 2 kg batch. Depyrogenation is widely used to remove pyrogens from contact materials.

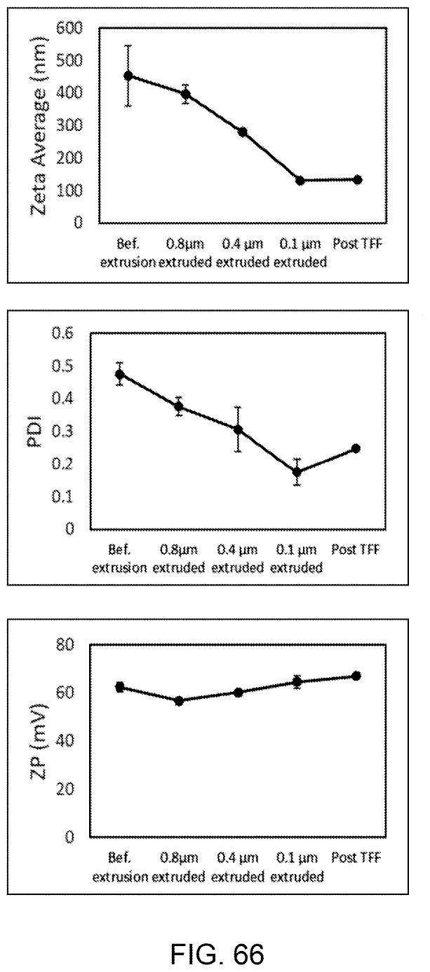

The result of the DLS investigation on MVI0022 is reported in Table 51 and FIG. 66.

TABLE 51
DLS and Zeta potential investigation of MVI0022

| SAMPLE | Z-Average | | Zeta Potential |
|---|---|---|---|
| MVI0022 | (nm) | PDI | (mV) |
| SiSaf ref. value | 108 +/− 0.50 | 0.089 +/− 0.01 | 39.3 +/− 0.95 |
| Before extrusion | 454.2 +/− 92.8 | 0.475 +/− 0.034 | 62.4 +/− 2.01 |
| 0.8 μm extruded | 396.9 +/− 27.76 | 0.376 +/− 0.027 | 56.6 +/− 1.41 |
| 0.4 μm extruded | 279.7 +/− 10.04 | 0.306 +/− 0.067 | 60.2 +/− 0.929 |
| 0.1 μm extruded | 132 +/− 2.27 | 0.175 +/− 0.041 | 64.5 +/− 2.57 |
| Post-TFF | 134.1 +/− 1.88 | 0.248 +/− 0.03 | 66.9 +/− 0.982 |

The result of the HPLC investigation on MVI0022 is reported in Table 52. Note that sample were diluted in MeOH as presented in section 2.4.3 pg. 32: pre-TFF sample was 2× diluted, whilst post TFF sample was 4× diluted in order to match the range of the calibration curve.

TABLE 52
HPLC investigation of MVI0022

| | | | LIPID CONTENT | | | | | |
|---|---|---|---|---|---|---|---|---|
| | | | | | DSPE- | | | |
| | | | DOPE | | mPEG2000 | | DOTAP-Cl | |
| | | | Conc. | | Conc. | | Conc. | |
| EXPERIMENT | | | mg/ml | Recovey | mg/ml | Recovey | mg/ml | Recovey |
| SiSaf ref. value | | | 0.725 | 100% | 0.145 | 100% | 0.730 | 100% |
| MVI0022 | Pre-TFF | 4 L | 0.698 | 96% | 0.132 | 91% | 0.688 | 95% |
| | Post-TFF | 2 L | 1.632 | 112% | 0.252 | 86% | 1.584 | 110% |

The result of the 1H-NMR investigation for MeOH quantification in MVI0022 is reported in Table 53.

TABLE 53
1H-NMR investigation on MVI0022

| | | | MeOH Content | |
|---|---|---|---|---|
| EXPERIMENT | | | ppm by volume | ppm by weight |
| MVI0022 | Pre-Filtr. | 4 L | 7.99 × 10⁴ | 7.91 × 10⁵ |
| | Post-Filtr. | 2 L | No peak detected | No peak detected |

Finally, the silicon recovery for the post TFF sample determined by ICP-OES was 2.9 mg/L.

Experimental observation on experiment MVI0022, indicates the following:

### DLS Measurements

Zeta average is slightly higher than what expected
   132 nm MVI0022 vs 108 nm ref value vs 78 nm average of small-scale batch
   Zeta average is largely maintained post TFF
PDI is in line with expected value pre TFF (0.175) with a slight increase post TFF (0.248)
Zeta potential is in line with the expected value pre and post TFF (64.5 mV)

### HPLC Measurements

Pre-TFF analysis showed high lipid recovery (95%) over the expected concentration
Post-TFF analysis showed high lipid recovery (100%) over the expected concentration

### 1H-NMR Measurements

No MeOH detected in the sample post TFF

### ICP-OES Measurements

Silicon recovery in line with the expected value

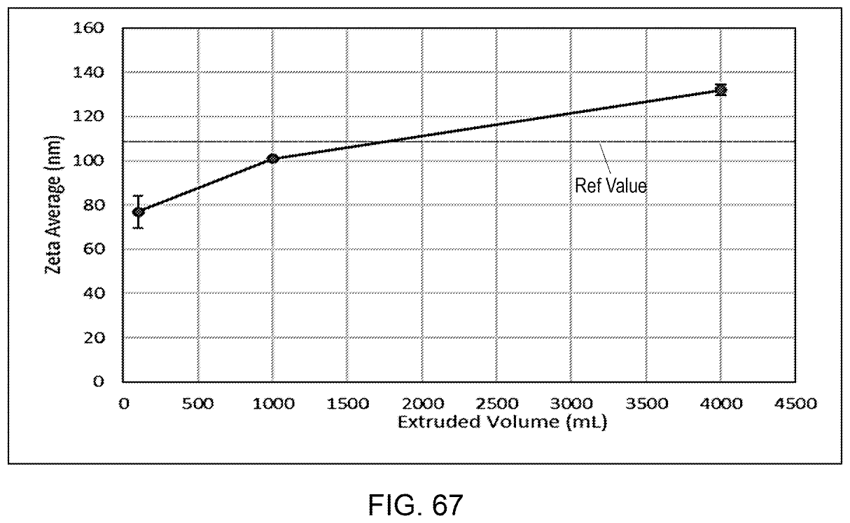

Notably, we observed a general trend of size increasing with high extrusion volumes, as reported in FIG. 67.

The extrusion membranes clogged during in flow extrusion due to presence of SiNPs aggregate, and this can be the source of the experimental variation for the Z-average. A prefiltration step right after SiNPs activation (0.8 μm filter or larger) can be used to avoid filter clotting.

Extrusion membranes were changed during extrusion due to pressure fluctuations. In flow extrusion through two parallel extrusion holder containing extrusion membranes of the same pore size can be considered for the next experiments to increase extrusion surface.

Time is a critical parameter for the extrusion process. Increasing the flow rate of the extrusion by changing pump/pump head will have a positive effect on the extrusion time (the one we are currently using has an intrinsic limit of 30 ml/min which decrease over time due to membrane clotting).

Finally, based on this experimental observation, the 0.5 m² TFF membranes can be used for 5 kg batch size.

### Summary of Examples

These examples investigate the feasibility of a scalable process for the manufacture of the micellar lipid particles of the invention. The feasibility of direct addition of activated SiNPs from methanol to an aqueous solution and the feasibility of direct addition of lipids into SiNPs, followed by in-flow extrusion and tangential flow filtration workup were investigated to generate a scalable process for the manufacture of the micellar lipid particles. Experimental results demonstrated the feasibility of these steps. DLS and Zeta potential features are in line with the expected features. The lipid recovery is 70% to 80% of the initial lipid concentration post extrusion. (DOPE: 79.2%, DSPE-mPEg: 76.6%, DOTAP-Cl: 70.4%). All these features are largely maintained after TFF. Additionally, TFF showed the successful removal of MeOH from all the investigated samples and the feasibility to concentrate the solution 2× to match the optimal concentration of components in the final product has also been demonstrated.

HPLC investigation of the lipid content revealed the presence of unknown peaks. The HPLC investigation over time of the samples indicated a decrease in concentration of lipids. However, the decrease in concentration shown by the HPLC measurements can be imputed to an instrumental variation due to the low measured concentration. Supporting this hypothesis increasing injection volume led to increased recovery of DSPE-mPEG lipid. Further investigation of this features by LC-MS for instance can be performed. silicon recovery showed large variability. Concentration via TFF work up is currently necessary to increase to match the ref. value for the silicon recovery.

Table 54, Table 55, summarizing the features of all analyzed sample are reported on the following pages.

TABLE 54
DLS and Zeta potential features for all the analyzed samples

| EXPERIMENT | | | | | Z-Average | | Z-Potential |
|---|---|---|---|---|---|---|---|
| Vol. | | | | | (nm) | PDI | (mV) |
| SiSaf ref. value | | | | | 108 +/− 0.50 | 0.089 +/− 0.01 | 39.3 +/− 0.95 |
| MVI0001 | | prehydrated + film | 10 | ml | 110 +/− 0.50 | 0.190 +/− 0.02 | 54.9 +/− 0.27 |
| | | hydration | | | | | |
| MVI0002 | | non-prehydrated + | 10 | ml | 129 +/− 0.91 | 0.125 +/− 0.02 | 64.9 +/− 2.80 |
| | | film hydration | | | | | |
| MVI0003 | | non-prehydrated + | 10 | ml | 102 +/− 0.17 | 0.135 +/− 0.01 | 56.0 +/− 0.81 |
| | | injection | | | | | |
| MVI0004 | | prehydrated + film | 50 | ml | 107.1 +/− 0.45 | 0.169 +/− 0.01 | 68.67 +/− 1.25 |
| | | hydration | | | | | |
| MVI0005 | A | non-prehydrated + | 50 | ml | 73.88 +/− 0.98 | 0.165 +/− 0.002 | 17.3 +/− 5.95 |
| | B | injection | | | 65.40 +/− 0.89 | 0.180 +/− 0.013 | 36 +/− 5.90 |
| | C | | | | 69.64 +/− 1.32 | 0.187 +/− 0.007 | 43.9 +/− 2.70 |
| MVI0006 | | non-prehydrated + | 10 | ml | 85.57 +/ 1.52 | 0.155 +/− 0.01 | 60.77 +/− 0.72 |
| | | injection | | | | | |
| MVI0007 | | non-prehydrated + | 50 | ml | 72.11 +/− 0.86 | 0.177 +/− 0.002 | 58.23 +/− 1.16 |
| | | injection | | | | | |
| MVI0008 | | non-prehydrated + | 50 | ml | 74.67 +/− 1.19 | 0.148 +/− 0.012 | 45.4 +/− 0.115 |
| | | injection | | | | | |
| MVI0009 | Pre- | non-prehydrated + | 50 | ml | 69.97 +/− 4.2 | 0.153 +/− 0.004 | 52.7 +/− 2.06 |
| | TFF | injection | | | | | |
| | Post- | | 50 | ml | 79.06 +/− 0.4 | 0.173 +/− 0.026 | 38.6 +/− 1.88 |
| | TFF | | | | | | |
| MVI0010 | Pre- | non-prehydrated + | 100 | ml | 84.64 +/− 1.64 | 0.221 +/− 0.015 | 48.3 +/− 1.31 |
| | TFF | injection | | | | | |
| | Post- | | 50 | ml | 76.59 +/− 1.13 | 0.177 +/− 0.005 | 50.0 +/− 0.75 |
| | TFF | | | | | | |
| MVI0011 | | non-prehydrated + | 100 | ml | 68.62 +/− 0.82 | 0.153 +/− 0.014 | 63.3 +/− 5.20 |
| | | injection + | | | | | |
| | | 0.8 μm filtration | | | | | |
| | | of the NP-Lipid | | | | | |
| | | solution pre- | | | | | |
| | | extrusion | | | | | |
| MVI0012 | | non-prehydrated + | 1 | L | 100.9 +/− 1.06 | 0.135 +/− 0.012 | 69.9 +/− 0.61 |
| | | injection | | | | | |
| MVI0013 | | non-prehydrated + | 100 | ml | 85.81 +/− 1.03 | 0.169 +/− 0.012 | 58.2 +/− 3.08 |
| | | injection + | | | | | |
| | | 0.8 μm NP | | | | | |
| | | filtration after | | | | | |
| | | activation | | | | | |
| MVI0014 | | non-prehydrated + | 100 | ml | 74.95 +/− 0.64 | 0.148 +/− 0.01 | 55.9 +/− 2.17 |
| | | injection using ⅕ | | | | | |
| | | of the amount of Si | | | | | |
| MVI0015 | Pre- | non-prehydrated + | 200 | ml | 99.41 +/− 1.04 | 0.142 +/− 0.01 | 64.4 +/− 2.01 |
| | TFF | injection without | | | | | |
| | Post- | the presence of | 100 | ml | 100.5 +/− 0.81 | 0.178 +/− 0.005 | 70.3 +/− 1.15 |
| | TFF | DSPE-mPEG2000 | | | | | |
| MVI0022 | Pre- | non-prehydrated + | 4 | L | 132 +/− 2.1 | 0.175 +/− 0.041 | 64.5 +/− 2.57 |
| | TFF | injection (2 Kg | | | | | |
| | Post- | Batch) | 2 | L | 134.1 +/− 1.88 | 0.248 +/− 0.03 | 66.9 +/− 0.98 |
| | TFF | | | | | | |

TABLE 55
HPLC content and lipid recovery for all the analyzed samples

| | | | | | LIPID CONTENT | | | | | |
|---|---|---|---|---|---|---|---|---|---|---|
| | | | | | | | DSPE- | | | |
| | | | | | DOPE | | mPEG2000 | | DOTAP-Cl | |
| | | | | | Conc. | | Conc. | | Conc. | |
| | | | | | mg/ml | Recovery | mg/ml | Recovery | mg/ml | Recovery |
| SiSaf ref. Values | | | | | 0.725 | 100% | 0.145 | 100% | 0.730 | 100% |
| MVI0008 | | non-prehydrated + | 50 | ml | 0.495 | 68% | 0.113 | 78% | 0.408 | 56% |
| | | injection | | | | | | | | |
| MVI0009 | Pre TFF | non-prehydrated + | 50 | ml | 0.580 | 80% | 0.120 | 82% | 0.510 | 70% |
| | Post TFF | injection | 50 | ml | 0.502 | 69% | 0.104 | 72% | 0.533 | 60% |
| MVI0010 | Pre TFF | non-prehydrated + | 100 | ml | 0.628 | 86% | 0.111 | 76% | 0.586 | 81% |
| | Post TFF | injection | 50 | ml | 1.242 | 85% | 0.212 | 73% | 1.134 | 78% |
| MVI0011 | | non-prehydrated + | 100 | ml | 0.554 | 76% | 0.103 | 71% | 0.503 | 69% |
| | | injection + | | | | | | | | |
| | | 0.8 μm filtration | | | | | | | | |
| | | of the NP-Lipid | | | | | | | | |
| | | solution pre- | | | | | | | | |
| | | extrusion | | | | | | | | |
| MVI0012 | | non-prehydrated + | 1 | L | 0.625 | 86% | 0.110 | 76% | 0.553 | 76% |
| | | injection | | | | | | | | |
| MVI0013 | | non-prehydrated + | 50 | ml | 0.717 | 98% | 0.130 | 90% | 0.700 | 96% |
| | | injection + | | | | | | | | |
| | | 0.8 μm NP | | | | | | | | |
| | | filtration after | | | | | | | | |
| | | activation | | | | | | | | |
| MVI0014 | | non-prehydrated + | 50 | ml | 0.614 | 84% | 0.118 | 82% | 0.579 | 80% |
| | | injection using ⅕ | | | | | | | | |
| | | of the amount of Si | | | | | | | | |
| MVI0022 | Pre TFF | non-prehydrated + | 4 | L | 0.698 | 96% | 0.132 | 91% | 0.688 | 95% |
| | Post TFF | injection (2 kg | 2 | L | 1.632 | 112% | 0.252 | 86% | 1.584 | 110% |
| | | batch) | | | | | | | | |
| SiSaf ref. Values for MVI0015 | | | | | 0.825 | 100% | 0 | 0 | 0.775 | 100% |
| MVI0015 | Pre TFF | non-prehydrated + | 200 | ml | 0.778 | 94% | 0 | 0 | 0.703 | 91% |
| | Post TFF | injection without the | 100 | ml | 1.062 | 64% | 0 | 0 | 0.916 | 59% |
| | | presence of DSPE- | | | | | | | | |
| | | mPEG2000 | | | | | | | | |

### Example 15—Storage Stability

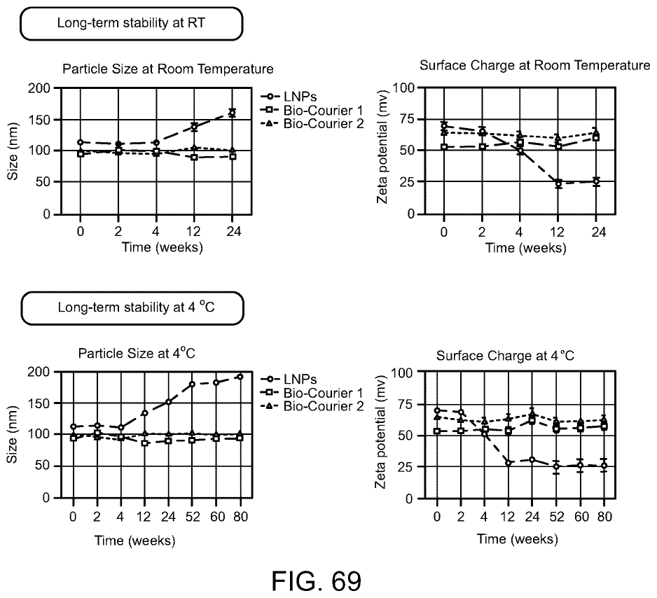

A study was carried out to compare the storage stability of hybrid lipid particles of the invention formulated with silicon, with lipid nanoparticles formulated without silicon or other particles of inorganic material. Particles were prepared according to methods described above for Biocourier MVI0012 and MVI0012. Prior art comparative particles (LNPs) were also made by the same method and with the same ingredience as MVI0012, but with particles of silicon or other particles of inorganic material. Particles were then stored at room temperature of 4° C. for many weeks. The size of the particles and their surface charge was determined at intervals and results are shown in FIG. 69. It can be seen that the positive charged surface of the particles of the invention endured for at least 24 weeks at room temperature and at least 80 weeks at 4° C. It is hypothesised that this positive surface charge (zeta potential of >30 mV) inhibited particle coalescence and it can be seen from FIG. 69 that in contrast to the LNP control, particle size remained constant thought the study period.

### Example 16—Size Stability Following Loading with RNA

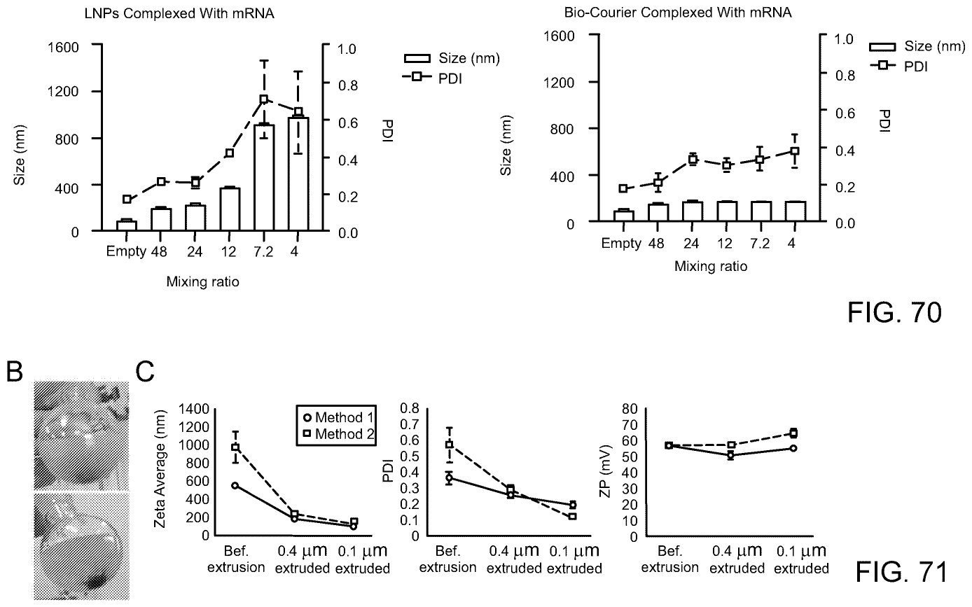

The particles of Example 15 were prepared without the an active API in order to demonstrate the stability of “empty” hybrid lipid particles of the invention. This example demonstrates that the addition of mRNA to the particles of the invention does not disrupt their stability. Empty biocourier MVI0012 and LNP particles were mixed with mRNA at various ratios and their size and monodispersity of size (PDI) was measured. The results are shown in FIG. 70 and show that unlike the situation with prior art LNP particles, particles of the invention retain their small sizer when complexed with mRNA at all ratios tested allowing higher loading with mRNA to be achieved without disrupting the particles.

### Materials and Methods for Examples 17 and 18

Silicon nanoparticles were sourced commercially as electrochemically etched powder (>98% purity; from American Elements, Inc., Los Angeles, CA; or The Porous Silicon Company, Salzburg, Austria). Trehalose and glycine (USP/PhEur specification) were purchased from Merck. The following lipids were supplied by Lipoid GmbH, Ludwigshafen, Germany: 1,2-dioleoyl-3-trimethylammonium propane chloride (DOTAP chloride; CAS No. 132172-61-3); 1,2-dioleoyl-sn-glycero-3-phosphoethanolamine (DOPE; CAS No. 4004-05-1); and N-(carbonyl-methoxypolyethylenglycol-2000)-1,2-distearoyl-sn-glycero-3-phosphoethanolamine, sodium salt (mPEG2000 DSPE; CAS No. 147867-65-0). All procedures used nuclease free water that was produced in house. Measurements of mean hydrodynamic particle size, PDI, and zeta potential were made using a Zetasizer Nano (Malvern Instruments, UK) as reported in previous studies (Baran-Rachwalska et al., 2020; Maurizi et al., 2023).

Formation of sshLNPs through lipid thin film hydration (FIG. 71). In Method 1, silicon nanoparticles were activated by suspending them in MeOH (20 mg/mL) and stirring for 0.5 h followed by slow evaporation, as previously described (Baran-Rachwalska et al., 2020). They were then added at 1 mg/mL into an aqueous solution of trehalose (1 mg/mL) and glycine (0.5 mg/mL), and the mixture was sonicated for 1 h at 50° C. Separately, lipid solutions of DOTAP chloride, DOPE, and if needed mPEG2000-DSPE were prepared at 5 mg/mL in MeOH and sonicated for 0.5 h at 40° C. Aliquots of these solutions (1.44 mL, 1.46 mL, and 0.29 mL, respectively) were transferred to a 10 mL round-bottomed flask and mixed. The solvent was removed by rotary evaporation for 0.5 h at 40° C. to generate the lipid thin film that was subsequently hydrated by adding 1 mL of the aqueous solution described above and incubating for 5 min at 60° C. Water was added up to a final volume of 10 mL (dehydration-rehydration method), then the mixture was extruded through 5×0.4 μm and 5×0.1 μm polycarbonate hydrophilic membranes (Whatman Nucleopore) arranged in series, using an Avanti Polar Lipid Extruder in combination with manual injection via a gastight Hamilton syringe. The membranes were prewashed with MeOH and water (20 mL each) prior to use. In Method 2, the MeOH was not evaporated from activated silicon nanoparticles before further use. Instead, an aliquot of the activated suspension was added directly into an aqueous trehalose/glycine solution to achieve the same final concentrations as above: 1 mg/mL SiNPs, 1 mg/mL trehalose, and 0.5 mg/mL glycine. Formation and extrusion of hybrid lipid particles were then carried out as above.

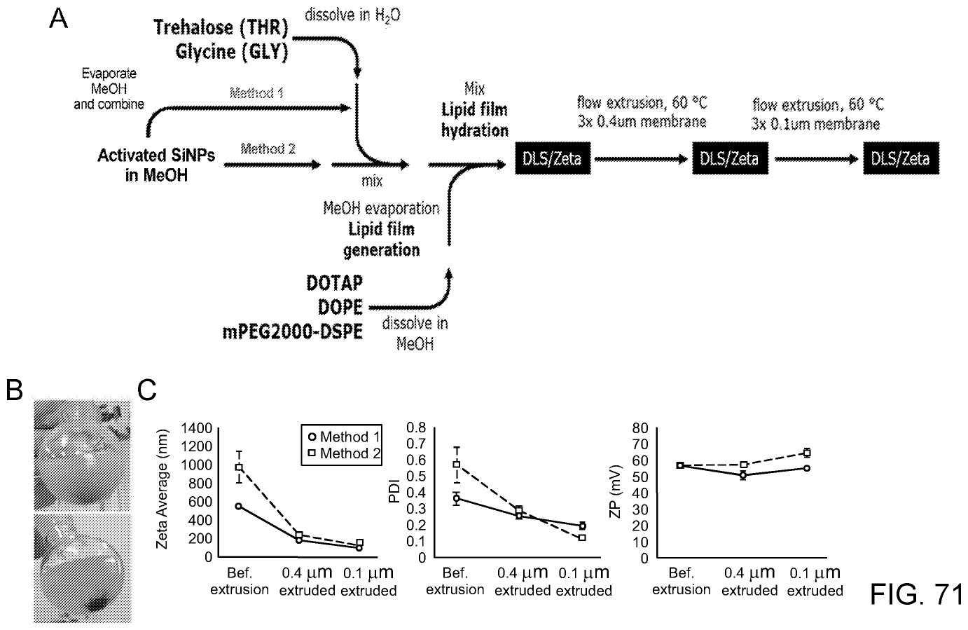

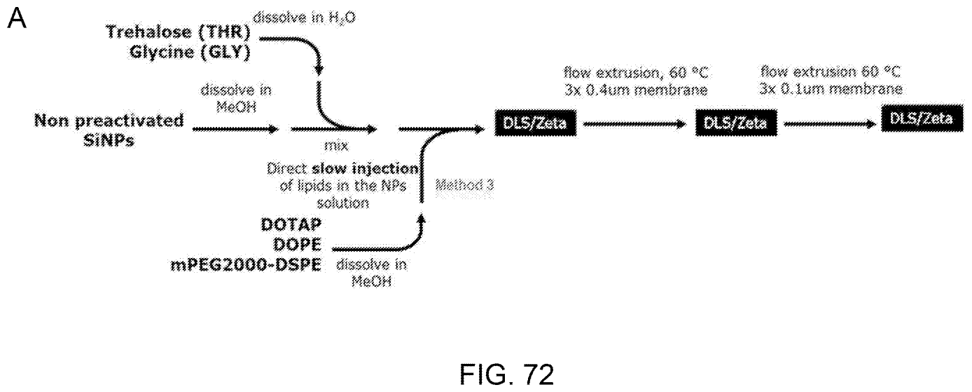

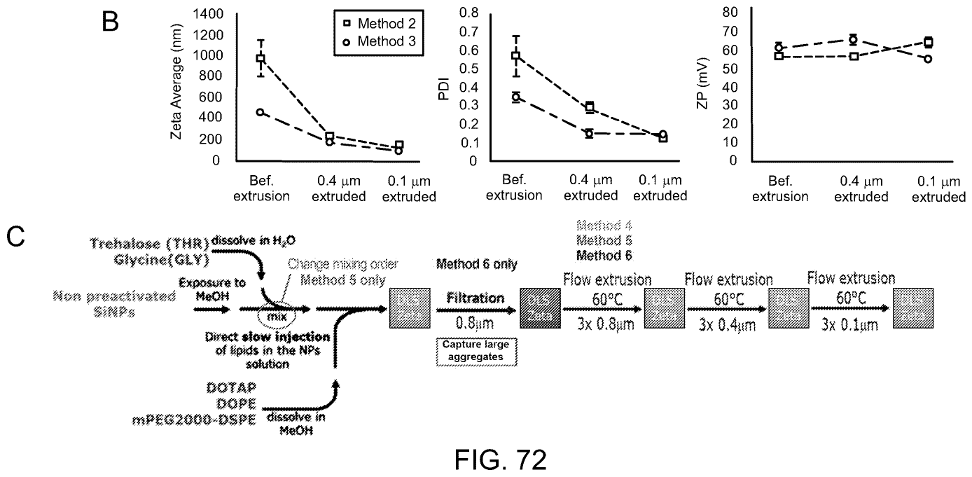

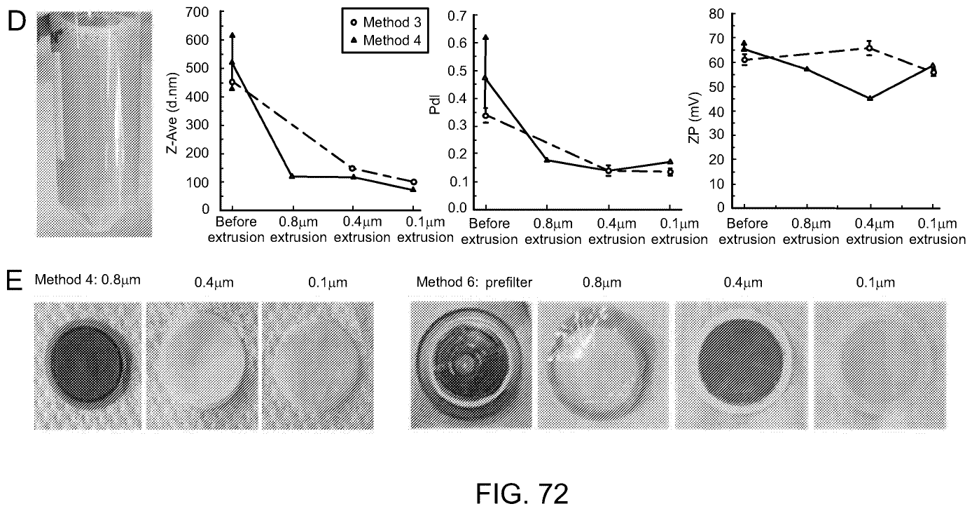

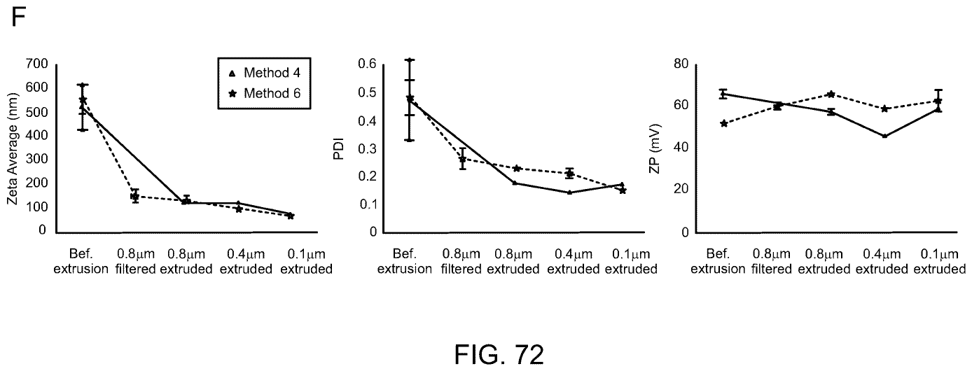

Formation of shybrid lipid particles through direct slow injection of lipids (FIG. 72). In Method 3, SiNPs (20 mg) were activated in MeOH (1 mL) as above then combined with a solution of trehalose (20 mg) and glycine (10 mg) in water (19 mL). The mixture was sonicated for 1 h at 50° C. Separately, lipid stock solutions were prepared and combined as in Method 1. The combined lipid solution was added to 1 mL of the aforementioned aqueous solution at 3.2 mL/min using a PSNE100 syringe pump (ProSense, Munich, Germany). The volume was made up to 10 mL with water and the solution was stirred for 0.5 h prior to extrusion, which as carried out as above.

Modified method with added 0.8 μm extrusion step (FIG. 72D). In method 4, an identical procedure was followed as for Method 3, except that it was performed at 5× the scale (i.e., final volume 50 mL) and also incorporated 3×0.8 μm polycarbonate hydrophilic membranes (Whatman Nucleopore) in series before the 3×0.4 μm and 3×0.1 μm membranes during the extrusion step.

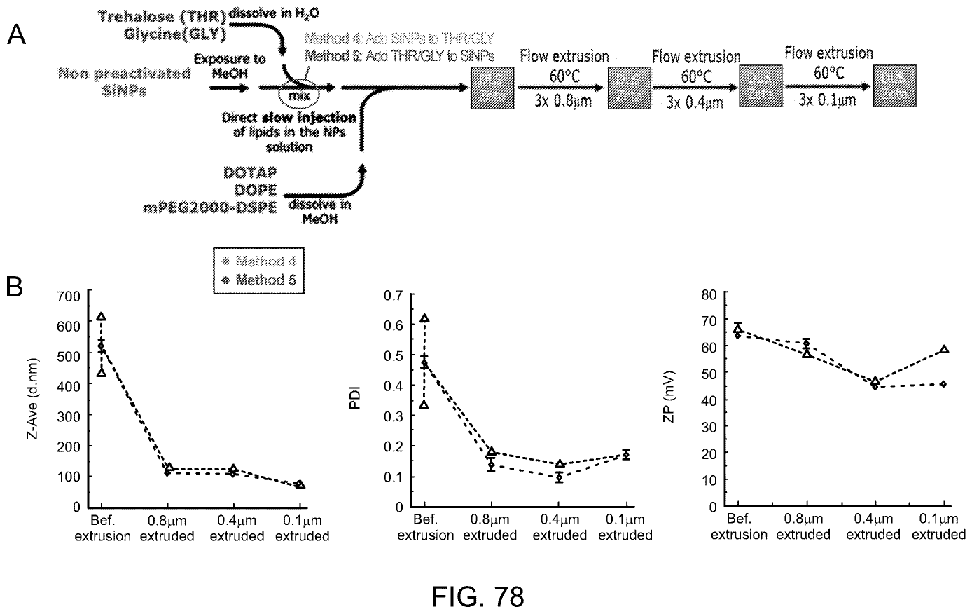

Other refinements to the small-scale protocol (FIGS. 72E, 78). In Method 5, the addition order used to prepare the aqueous solution was varied. The preprepared aqueous solution of trehalose/glycine was added directly to the activated methanolic suspension of SiNPs, rather than the other way round as before (in Method 4). The final concentrations of silicon nanoparticles (1 mg/mL), trehalose (1 mg/mL) and glycine (0.5 mg/mL) in the intermediate aqueous solution were unchanged, and the rest of the procedure was performed as before. Method 6 was carried out at double the scale of Method 4 (i.e., final volume 100 mL) and introduced two new variations. First, the lipid stock solutions were prepared at 10 mg/mL rather than 5 mg/mL, then aliquots of the DOTAP chloride (7.20 mL), DOPE (7.30 mL), and mPEG2000-DSPE (1.45 mL) were combined and added to the preprepared aqueous solution as above. After adjusting the volume to 100 mL with water, the mixture was prefiltered through a 0.8 μm hydrophilic polyethersulfone (PES) syringe filter and extruded as in Method 4.

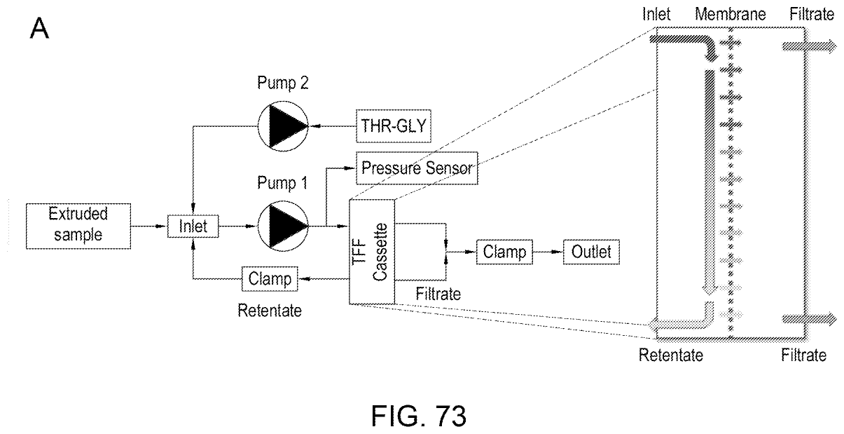

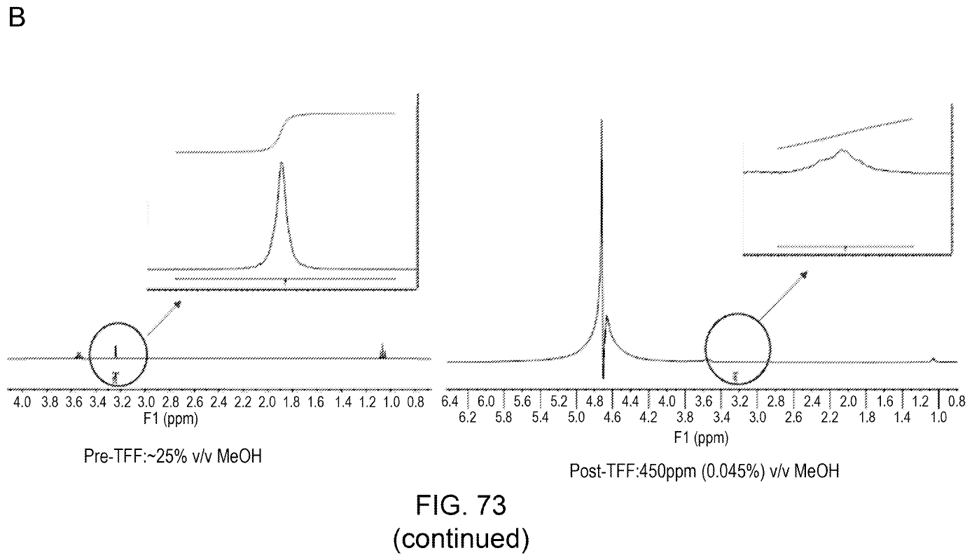

Diafiltration using a TFF setup (FIG. 73). A SIUS PD 0.02 m² (LP) HyS 100 kD cassette (XP100LP2L; Repligen, Waltham, MA) was used for volumes up to 100 mL, and a SIUS 0.5 m² 100 kD TFF cassette (NC1095082; Fisher Scientific, Waltham, MA) was employed for larger volumes. After an initial ultrafiltration step where indicated (typically to 0.5× input volume), samples were diafiltered against 10 volumes of an aqueous solution containing 0.1 mg/mL trehalose and 0.05 mg/mL glycine, to preserve the concentration of these two excipients while removing MeOH and unbound lipids. This was achieved by using a second pump to introduce the trehalose/glycine solution into the flow path at the same flow rate as the main system pump (FIG. 73A). FIGS. 73C and 73D show results for repeat runs of Method 5 followed by TFF with diafiltration only, or TFF with ultrafiltration (to half of the initial volume) followed by diafiltration.

Methanol content analysis. A calibration curve was constructed by measuring ¹H NMR peak area (δ=3.34 ppm; Gottlieb et al., 1997) in standard solutions of known MeOH concentration in water containing 10% D₂O: 50, 100, 300, and 500 ppm (v/v; FIG. 73B, inset). Pre-TFF and post-TFF samples were analyzed in H₂O/D₂O (90:10) and the MeOH content estimated by reference to the calibration curve. Due to their higher MeOH content, the pre-TFF samples were diluted by a factor of 1:1,000 before acquiring NMR spectra.

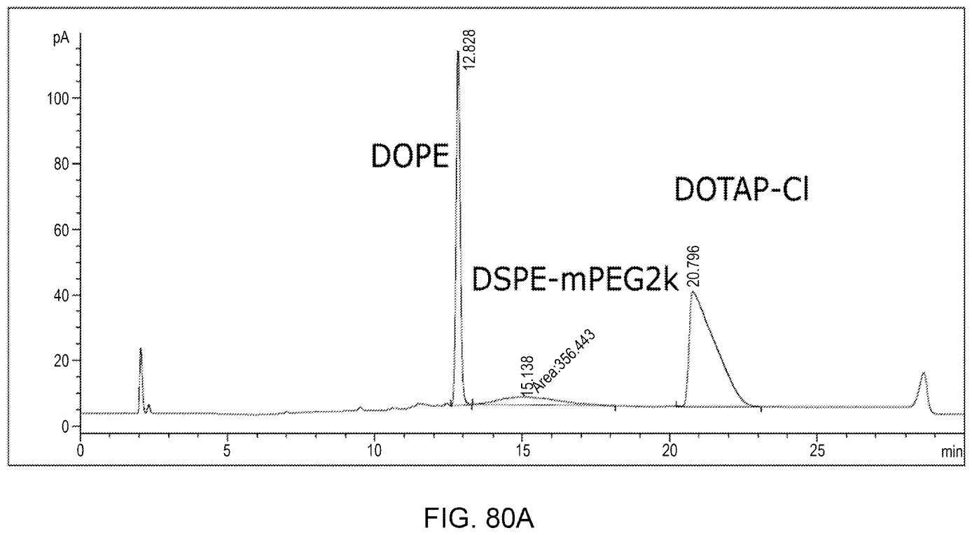

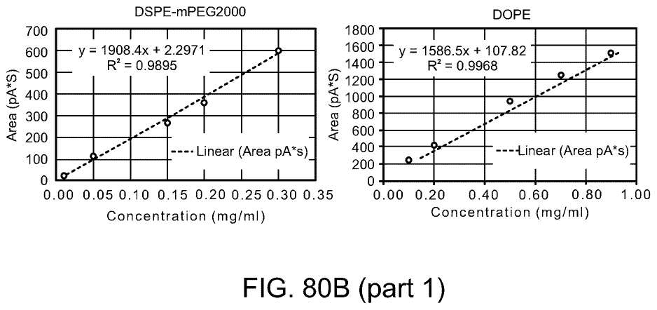

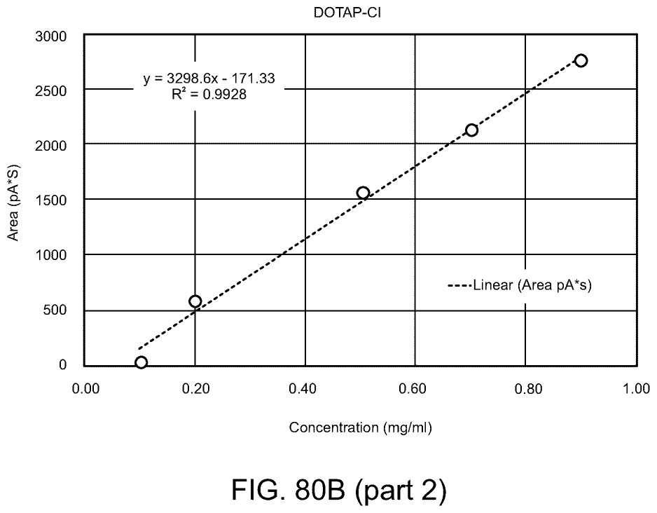

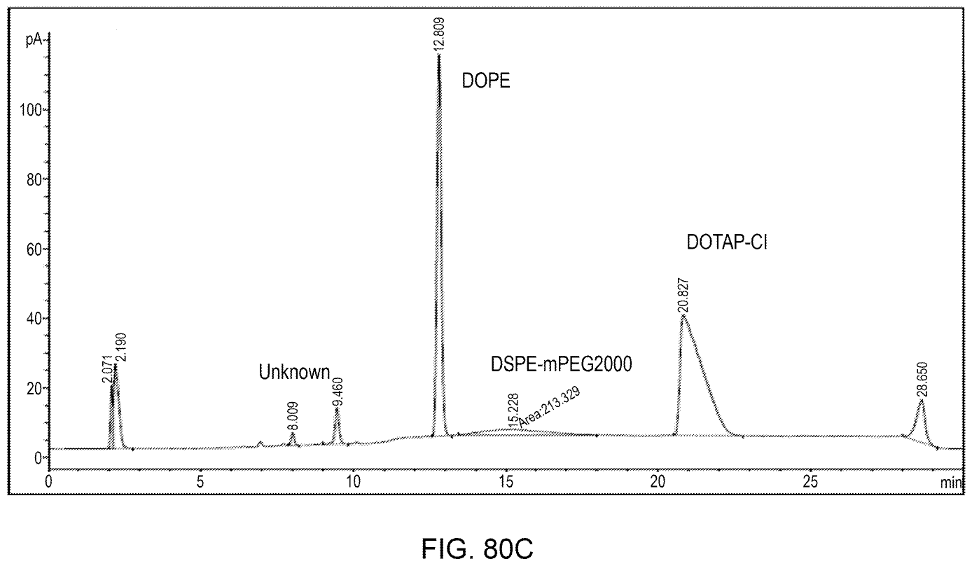

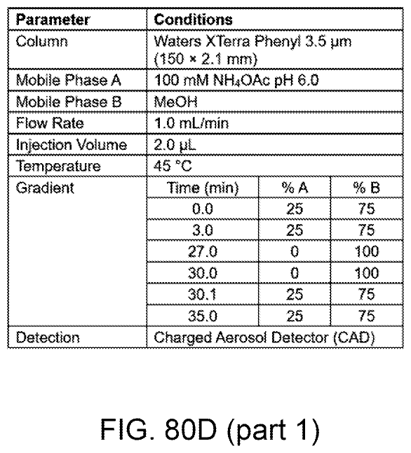

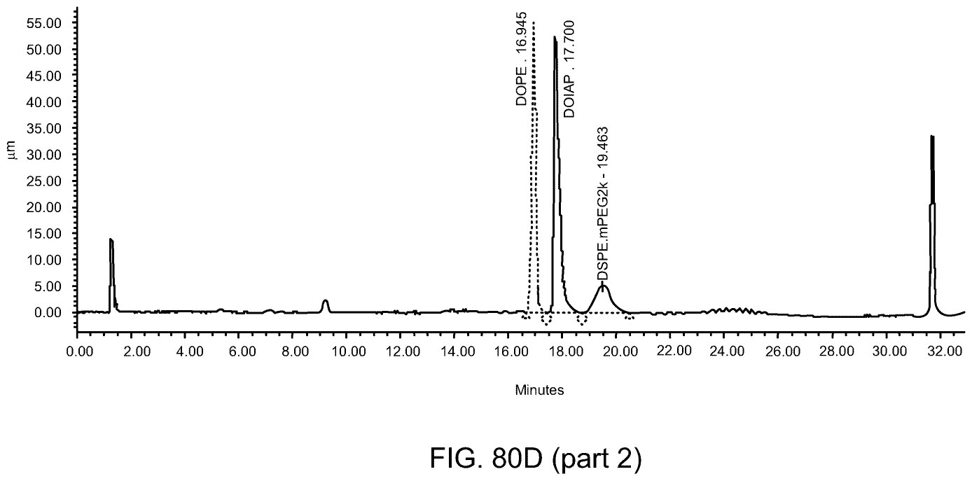

Lipid recovery analysis. Quantification of lipids relied on HPLC analysis using a Waters XBridge BEH Phenyl Column (130 Å, 5 μm, 4.6×150 mm) in conjunction with a charged aerosol detector (CAD). Buffer A was 40 mM NH₄OAc and Buffer B was 100% MeOH, and the following elution gradient was used: 25% A for 1 min, ramp to 5% A over 6 min, hold at 5% A for 18 min, ramp to 25% A over 0.1 min, then hold at 25% A for 4.9 min (flow rate 1 mL/min). To enable quantification, calibration curves were constructed for DOTAP chloride, DOPE, and mPEG2000-DSPE (which exhibited retention times of 12.8 min, 15.1 min, and 20.8 min, respectively; see FIG. 80). For DOTAP chloride and DOPE, standard solutions were prepared at 0.1, 0.2, 0.5, 0.7, and 0.9 mg/mL; and for mPEG2000-DSPE, the reference solutions had concentrations of 0.01, 0.05, 0.15, 0.2, and 0.3 mg/mL. Peak area was plotted against concentration to enable determination of lipid concentrations in test samples. Lipid recovery was calculated as the ratio of the observed concentration to the theoretical concentration if all lipid had been incorporated into the sshLNPs, expressed as a percentage.

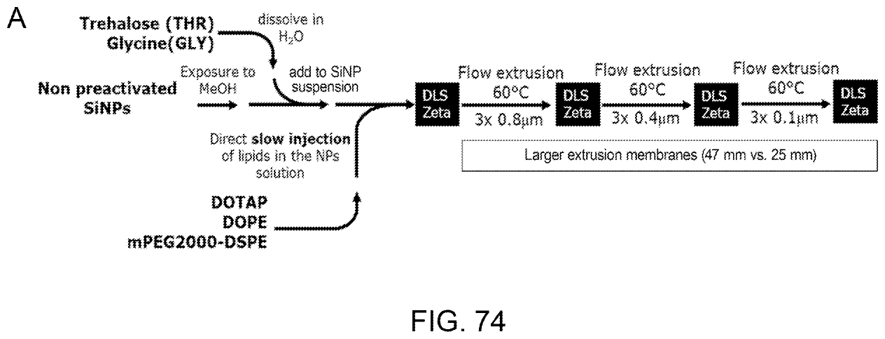

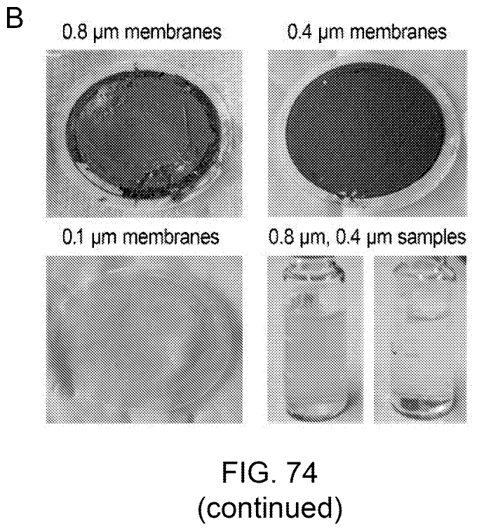

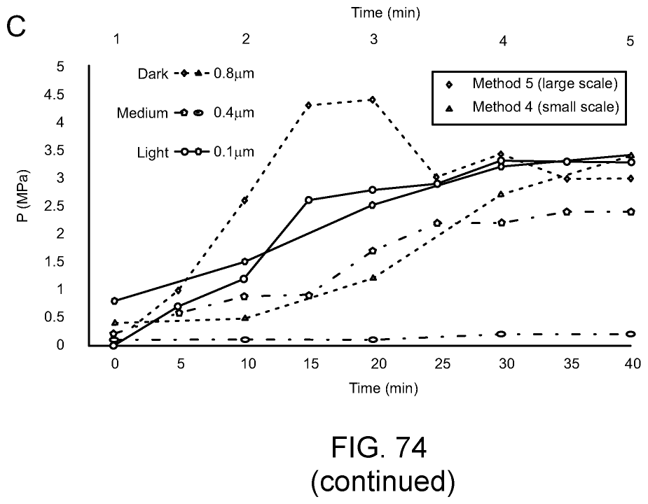

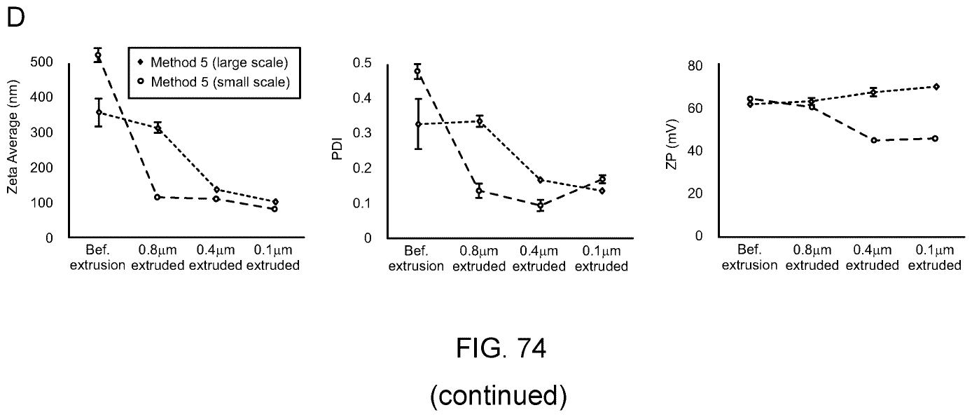

First large-scale test run to produce sshLNPs (1 L scale, FIG. 74). Silicon nanoparticles (100 mg) were suspended in MeOH (5 mL) in a sterile 50 mL Falcon tube and activated by incubating for 0.5 h at room temperature. Then, a solution of trehalose (100 mg) and glycine (50 mg) in water (95 mL) was added directly to the silicon nanoparticle suspension and the mixture was sonicated for 1 h at 50° C. Lipid stock solutions were prepared by dissolving DOTAP chloride (750 mg), DOPE (750 mg), and mPEG2000-DSPE (200 mg) in MeOH at 10 mg/mL and sonicating the resultant mixtures for 0.5 h at 40° C. Then, aliquots of the DOTAP chloride (72.5 mL), DOPE (73.0 mL), and mPEG2000-DSPE (14.5 mL) solutions were combined (i.e., to achieve final concentrations of 0.725, 0.730, and 0.145 mg/mL, respectively). The preprepared aqueous solution (100 mL) was transferred to a sterile 1 L bottle and the methanolic mixed lipid solution (160 mL) was injected at 6 mL/min using a Knauer K501 HPLC pump. After addition was complete, the volume was made up to 1 L with water and in-flow extrusion was performed using 3×0.8 μm, 3×0.4 μm, and 3×0.1 μm polycarbonate hydrophilic membranes (47 mm diameter, Whatman Nucleopore) arranged in series as above, at 60° C. and using a flow rate of 85 mL/min.

Second large-scale test run to product hybrid lipid particles (2 L scale, FIG. 6). SiNPs (400 mg) were activated in MeOH (20 mL) as described above. A solution of trehalose (400 mg) and glycine (200 mg) in water (380 mL) was prepared in a sterile bottle, then the suspension of activated SiNPs was added directly and the resultant dispersion sonicated for 1 h at 50° C. Stock solutions of DOTAP chloride (3.00 g), DOPE (3.00 g), and mPEG2000-DSPE (600 mg) were prepared at 10 mg/mL in MeOH as described above. The aqueous solution (400 mL) was transferred to a 5 L sterile bottle, then the combined lipid mixture (290 mL, 292 mL, and 58 mL, respectively, of the DOTAP chloride, DOPE, and mPEG2000-DSPE stock solutions; total volume 640 mL) was injected into the bottle at 6 mL/min using an HPLC pump. Water was added up to a final volume of 4 L and stirring was continued until the mixture was homogenous. The crude mixture was stored at 4° C. overnight, then in-flow extrusion was performed as described for the previous example. The TFF setup (as shown in FIG. 73A, using a SIUS 0.5 m2 100 kD cassette) was first depyrogenated with 0.2 M NaOH for 1 h, then the 4 L sample was concentrated to 2 L via ultrafiltration and diafiltered against 10 volumes (i.e., 20 L) of 0.1 mg/mL trehalose and 0.05 mg/mL glycine in water. Final and intermediate hybrid lipid particle samples were analyzed using procedures as described above, except that for lipid content analysis, pre-TFF and post-TFF samples were diluted 1:2 and 1:4, respectively, with MeOH to match the range of the calibration curves.

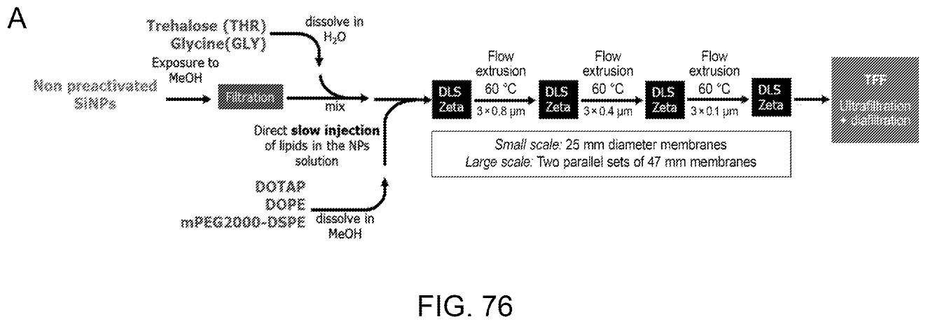

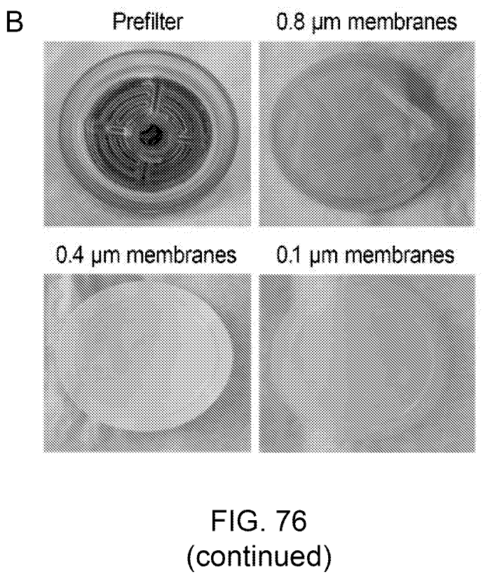

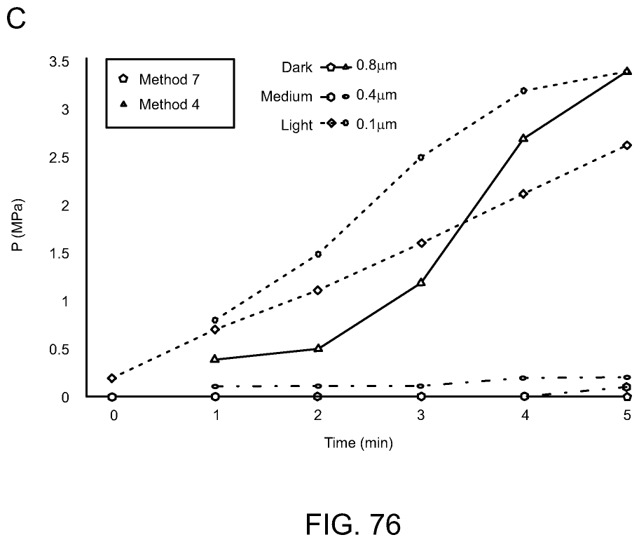

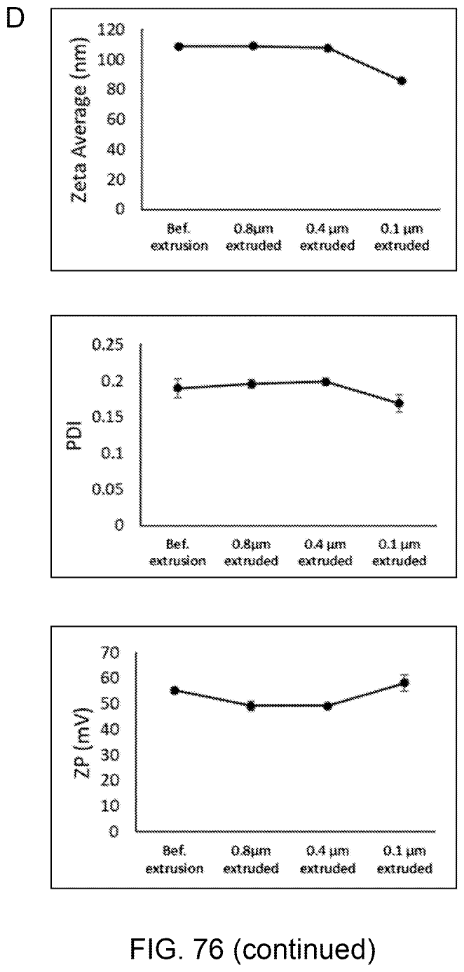

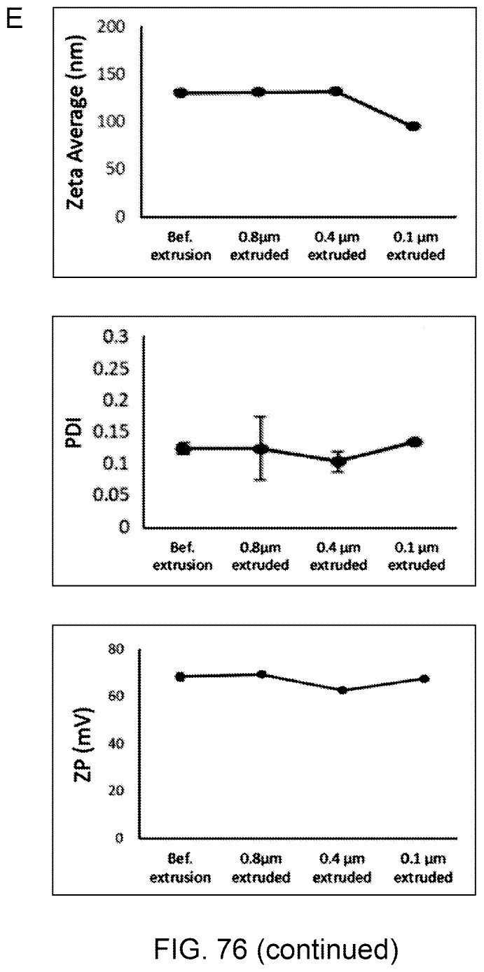

Modified procedure for hybrid lipid particles including prefiltration of aggregates (FIG. 76D). Method 7 was the same as Method 5 (50 mL scale), except for an additional prefiltration step of the activated silicon nanoparticles. Specifically, silicon nanoparticles (20 mg) were suspended in MeOH (1 mL) and activated by stirring for 0.5 h at room temperature, then the mixture was manually filtered through a 0.8 μm hydrophilic PES syringe filter. A preprepared solution of trehalose (20 mg) and glycine (10 mg) in water (19 mL) was added directly to the silicon nanoparticle suspension and the dispersion was sonicated for 1 h at 50° C. Separately, stock solutions of DOTAP chloride, DOPE, and mPEG2000-DSPE were prepared at 10 mg/mL in MeOH with sonication for 0.5 h at 40° C. Aliquots of these solutions (3.63 mL, 3.65 mL, and 0.73 mL, respectively) were combined, and the mixed lipid solution was injected into a 5 mL aliquot of the aqueous solution at 3.2 mL/min using a syringe pump. The volume was made up to 50 mL with water and stirred for 0.5 h at room temperature prior to extrusion, which was carried out as for Method 4.

Demonstration of a large batch with aggregate prefiltration (1 L scale, FIG. 76E). Silicon nanoparticles (100 mg) were activated by stirring in MeOH (5 mL) for 0.5 h at room temperature. To remove large aggregates, the suspension was manually filtered through three successive 0.8 μm hydrophilic PES syringe filters. The resultant sample was combined with a preprepared solution of trehalose (100 mg) and glycine (50 mg) in water (95 mL), with vigorous magnetic stirring (500 rpm) for 1 h at 50° C. Separately, stock solutions of DOTAP chloride (750 mg), DOPE (750 mg), and mPEG2000-DSPE (160 mg) were prepared at 10 mg/mL in MeOH with sonication for 0.5 h at 40° C. Aliquots of these solutions (72.5 mL, 73.0 mL, and 14.5 mL, respectively) were combined, then the mixed lipid solution was injected into the aqueous solution (in a 1 L polycarbonate container) at 6 mL/min using a Knauer BlueShadow 80P HPLC pump. The volume was made up to 1 L with water (added using the same pump at 100 mL/min, with stirring), and stirring was continued for 0.5 h at room temperature for homogenization. In-flow extrusion was performed using two sets of extrusion membranes (three each of 0.8 μm, 0.4 μm, and 0.1 μm pore size) arranged in parallel, at 60° C. and a flow rate of 85 mL/min. After the TFF setup was depyrogenated with 0.2 M NaOH for 1 h and rinsed with water, methanol was removed by diafiltration as outlined above.

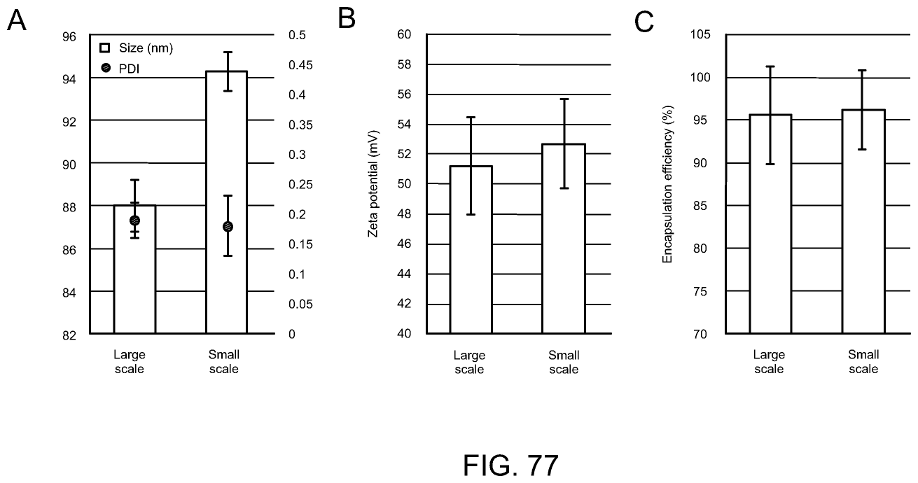

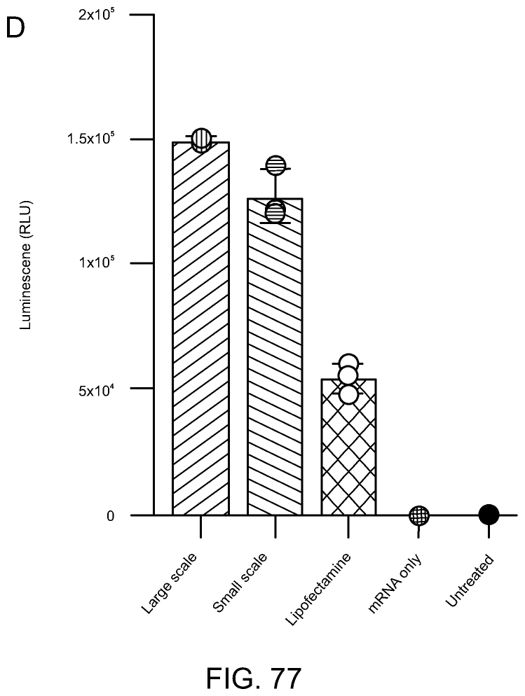

Assessment of RNA encapsulation efficiency (FIG. 77C). The Quant-iT KiboGreen Assay Kit (Fisher Scientific, UK) was used in accordance with the manufacturer's instructions, as reported previously (Maurizi et al., 2023). For analysis, a Varioskan LUX microplate reader (Thermo Fisher Scientific) was used (excitation 480 nm, emission 520 nm).

### Example 17—Removal of Evaporation Steps

A traditional method of manufacturing lipid nanoparticles may include at least 2 solvent evaporation steps. Firstly, inorganic particles may be activated with a solvent, which may be evaporated in a first evaporation step. Secondly, the inorganic particles and the lipid may be mixed in a solvent and the solvent evaporated to form a thin lipid film. It was previously thought that the first evaporation step was necessary to ensure proper activation of the inorganic particles. It was previously thought that the second evaporation step leading to the formation of a thin lipid film, and the subsequent hydration of that film was necessary for the correct hydration and formation of lipid nanoparticles. This example demonstrates each or both of the evaporation steps may be disgarded.

As noted above, small-scale production of lipid nanoparticles may be carried out using a workflow based on lipid thin film hydration followed by flow extrusion. Such a method is shown diagrammatically in FIG. 71A, labelled as “method 1”. Two successive in-flow extrusion steps through membranes with successively smaller pore size (0.4 μm and 0.1 μm) were used to reduce the operating pressure on the membranes and ensure that the final particles had the desired dimension. Trehalose (1 mg/mL) and glycine (0.5 mg/mL) were also included as excipients in the aqueous phase since it has been found the further stabilize the the hybrid lipid particles, presumably through coordination to the silicon nanoparticles (SiNPs).

The presence of trehalose and glycine favor the dispersibility of the silicon particles in the mixed hydroalcoholic environment, provided the ratio is appropriate. The amino acid excipient is also included to modulate the rate of hydrolysis of silicon, promoting formation of orthosilicic acid rather than insoluble polymeric silicon species (see WO2011012867A1 for further details of that).

To develop this method for large-scale application, the two solvent evaporation steps needed to be eliminated; namely, removal of MeOH from the silicon nanoparticle suspension after activation, and from the dissolved lipids used to create the thin film. The first of these steps was removed by mixing the activated silicon nanoparticles suspension directly into the aqueous trehalose/glycine solution prior to lipid thin film hydration. Such a method is shown diagrammatically in FIG. 71A, labelled as “method 2”. This approach resulted in an initial hybrid lipid nanoparticles suspension with visibly more sediment than before (see FIG. 71B, lower image; upper image shows method 1), but this difference was removed by producing particles with a lower polydispersity index (PDI) and slightly higher zeta potential after extrusion (see FIG. 71C). preferably, finished hybrid lipid nanoparticles should have a zeta potential of +50 to +70 mV, indicating good to excellent colloidal stability and minimal tendency for aggregation (due to charge repulsion). It can be seen that the removal of the first evaporation step does not prevent hybrid lipid particles from being produced which have acceptable zeta potential and polydispersity.

In order to demonstrate that the lipid thin film evaporation and subsequent hydration step can be removed without detriment, direct slow injection of the methanolic lipid solution into an aqueous silicon nanoparticle suspension was attempted (shown diagrammatically in FIG. 72A, labelled as “method 3”). Less sedimentation was seen with this approach (FIG. 72B), and the resultant sample of hybrid lipid particles showed comparable average size (102±0.17 nm), PDI (0.135±0.01), and zeta potential (56.0±0.81 mV) to previous batches (FIG. 72B). This established feasibility for the more scalable direct injection mixing technique. To ensure reproducibility, we introduced two in-process checks (IPCs) into the method (FIG. 72A): a visual check for complete dissolution of lipids before mixing (IPC 1), and specified reference ranges for post-extrusion dynamic light scattering (DLS) parameters: i.e., average hydrodynamic size 75-140 nm, PDI 0.1-0.2, and zeta potential +50 to +70 mV (IPC 2). If required, the 0.1 μm extrusion step is repeated following IPC 2.

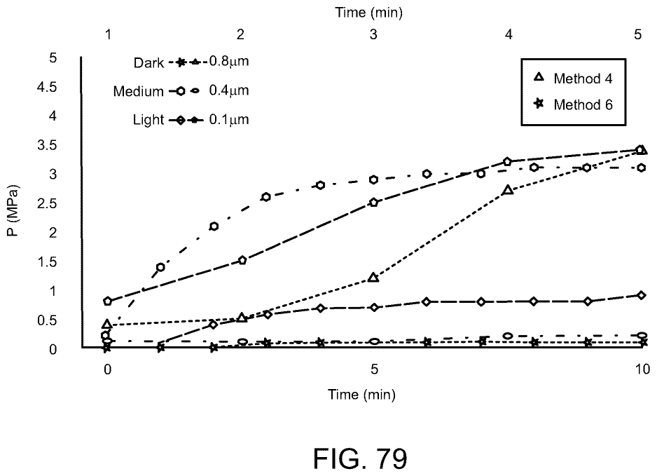

Other method adjustments were investigated before scale-up (FIG. 72C, labelled as “methods 4 to 6”). Most notably, an additional in-flow extrusion step (0.8 μm pore size) was trialed to remove larger silicon nanoparticle aggregates and successfully led a reduction of average size of the final hybrid lipid particles (FIG. 72D, “method 4”). Adding the trehalose/glycine solution to the activated methanolic suspension of silicon nanoparticles, rather than the other way round (FIG. 72C “method 5”, FIG. 78), did not affect the results. Including a 0.8 μm prefiltration step after initial hybrid lipid particle formation reduced buildup of aggregates on the extrusion membranes (FIG. 72E, “method 6”), significantly lowering operating pressure on the 0.8 μm membrane (FIG. 79) and essentially leading to sshLNPs with unaltered properties (FIG. 72F).

For commercial manufacturing of a pharmaceutical product, the organic solvent (i.e., MeOH) must preferably be removed from the finished product such that any residual levels are below regulatory limits (e.g., <3,000 ppm in pharmaceutical products in Europe; European Medicines Agency, 2022). For this purpose, tangential flow filtration (TFF) may be used as depicted diagrammatically in FIG. 73A. Initial feasibility studies (up to 100 mL scale) used a TFF cassette with 100 kDa MWCO and 0.02 m² membrane surface area. Since trehalose and glycine are also removed from the solution under these conditions, a second pump was used to infuse an aqueous diafiltration solution of 0.1 mg/mL trehalose and 0.05 mg/mL glycine at a constant flow rate matching that of filtrate generation. Diafiltration against 10 volumes of this solution lowered the MeOH content from ~20% v/v after extrusion to well within acceptable regulatory limits (~200 ppm based on ¹H NMR) whilst maintaining the intended levels of trehalose/glycine. The final process included an initial ultrafiltration step to reduce the input volume by 50% before performing diafiltration, to produce a finished hybrid lipid nanoparticle suspension with the desired silicon concentration (see below) and low residual MeOH levels (~450 ppm; FIG. 73B).

After TFF, the DLS properties of the extruded sshLNPs were largely maintained. A HPLC method was used to measure the extent of lipid recovery (FIG. 80). The results showed a notable drop in lipid content when applying diafiltration only, of around 10% recovery for each lipid. However, this effect was largely mitigated by the adoption of a modified TFF procedure that combines both ultrafiltration and diafiltration. The silicon content of final hybrid lipid nanoparticle samples was also measured using inductively coupled plasma-optical emission spectroscopy (ICP-OES). For the batch subjected to TFF with diafiltration only, the silicon content was 1.19 mg/L, compared with a target value 2.1 mg/L based on product prepared using the original lipid thin film hydration method (FIG. 71A). The aforementioned analytical methods were used to guide further optimization efforts on moving toward scale-up. They may also be used for phase appropriate quality control during commercial manufacture. As an alternative to a ¹H NMR method for determining residual MeOH content, a headspace GC assay may be used (Maurizi et al., 2023).

### Example 18—Large-Scale Test Run

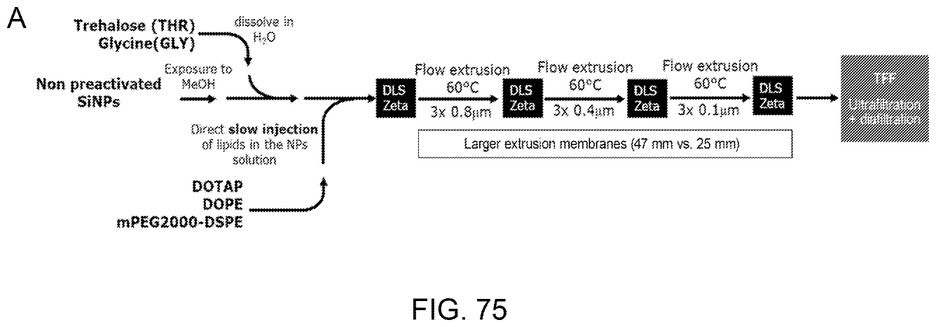

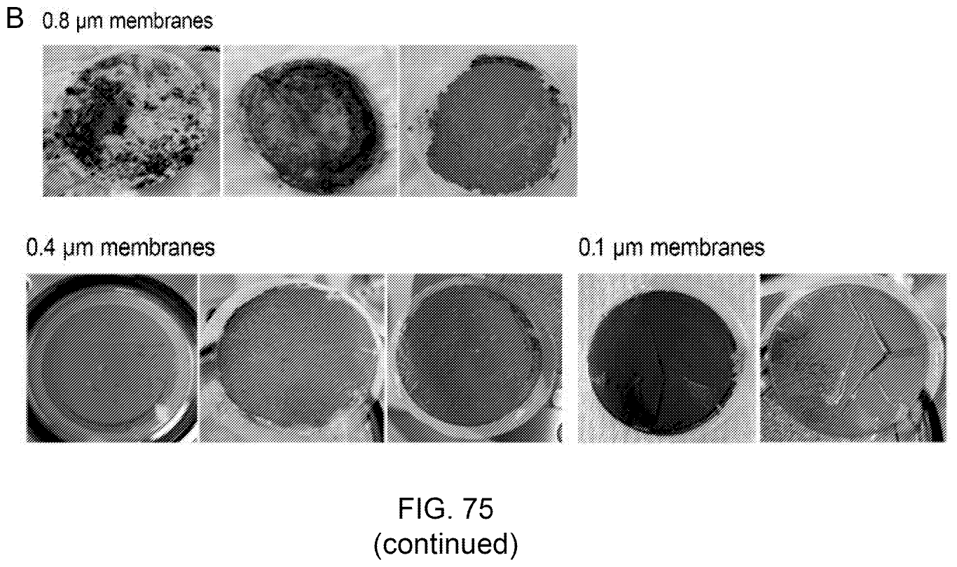

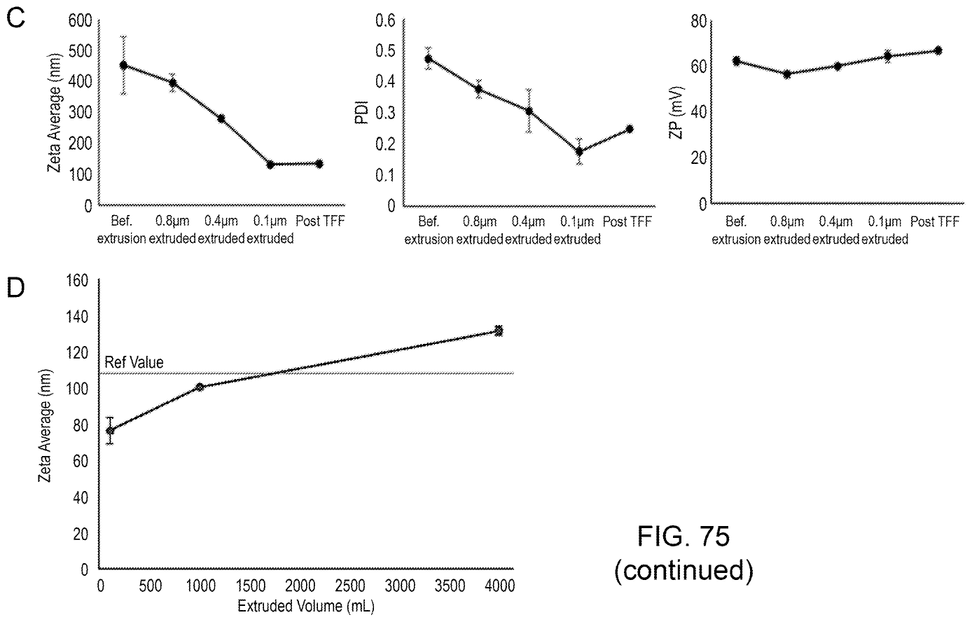

The first large-scale test run (shown diagrammatically in FIG. 74A) used a protocol incorporating the three in-flow extrusion steps (i.e., a scale-up of method 4 of example 17 using the modification introduced in method 5 of example 17) but omitting TFF at this stage. Lipid stock solutions were now prepared at 10 mg/mL rather than 5 mg/mL as before, to limit the volume of MeOH in the system. On a 1 L scale, the main issue encountered was greater accumulation of insoluble aggregates on the extrusion membranes (FIG. 74B), even though larger membranes were used (47 mm diameter vs. 25 mm previously). This resulted in higher operating pressures during extrusion (FIG. 74C), although the DLS properties of the sample were not adversely affected (FIG. 74D). The production of a 2 L batch was demonstrated by using a two-stage TFF protocol (FIG. 75A), but in this case using a cassette with larger membrane surface area (0.5 m²). As before, the main issue encountered was during extrusion, where the membranes became saturated with insoluble aggregates (FIG. 75B) and had to be changed midway through the procedure due to pressure fluctuations. The post-extrusion sample exhibited suboptimal visual and DLS characteristics (Figure FIG. 75C), but closer process monitoring revealed that that average particle size had gradually increased with extrusion volume (FIG. 75D) and only exceeded the reference range after around 1.5 L of the total volume of 4 L had been processed. Following TFF, residual MeOH was below detection limits. When a 100 mL aliquot of the pre-TFF sample was subjected to an additional extrusion step through 3×0.1 μm membranes, the average particle size dropped from 129±0.44 nm to 105±0.38 nm, and the PDI from 0.17±0.02 to 0.11±0.01 (both well within reference ranges), confirming overloading of the extrusion membranes as the problem during processing of the large batch and demonstrating that such problems may be easily remedied by use of additional extrusion.

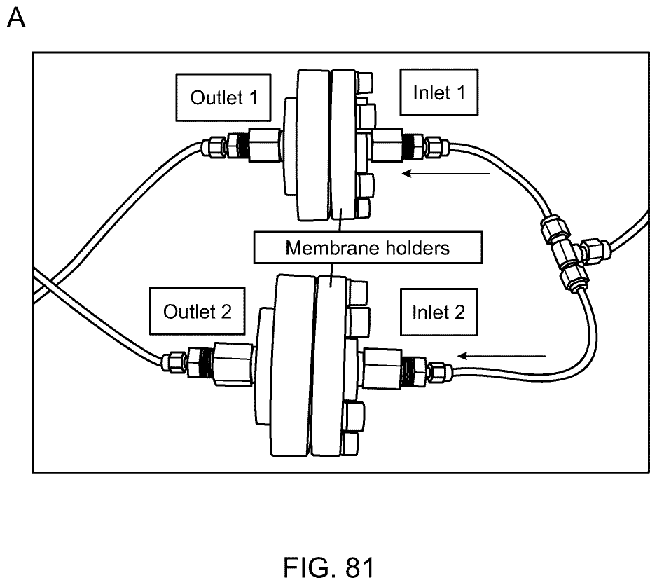

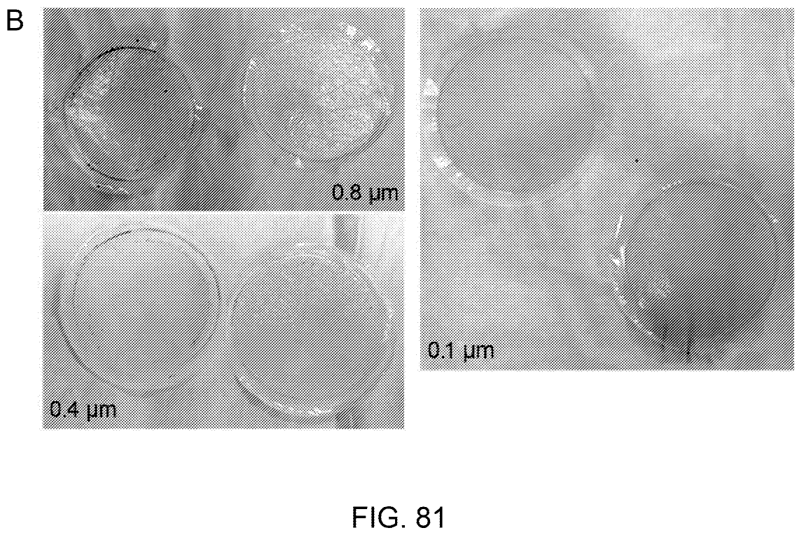

In view of these results, we modified the procedure to reduce sedimentation during sshLNP formation. In a small-scale test run omitting TFF, when the activated suspension of SiNPs was filtered prior to mixing with the trehalose/glycine solution (FIG. 7A, Method 7), aggregates were successfully removed and no significant deposition of insoluble material on the extrusion membranes was observed (FIG. 7B). Operating pressure was lower during extrusion (FIG. 7C) and DLS properties of the resultant sshLNPs were well within reference ranges (FIG. 7D). To implement this method at scale, two sets of the larger 47 mm extrusion membranes were used, arranged in parallel (FIGS. 76A and 81), successfully enabling production of a 1 L batch of sshLNPs via the optimized protocol (FIG. 76E). In the current implementation, a final 0.2 μm filtration step is additionally used for bioburden reduction.

Following development of the kilogram-scale manufacturing protocol, it was also essential to establish equivalent properties between sshLNPs produced by the original lipid thin film hydration technique (Method 2) and the optimized large-scale process (Method 7). We found that scale-up led to slightly smaller particles, on average (FIG. 77A), but the PDI and zeta potential were essentially identical (FIGS. 77A and 77B). Both batches showed excellent mRNA encapsulation efficiency, as determined by the RiboGreen assay (FIG. 77C). In cellular transfection experiments, sshLNPs produced by either route efficiently introduced an mRNA encoding firefly luciferase (fLuc) into HEK293 cells, and significantly higher transgene expression was observed after 24 h compared with Lipofectamine 3000 as reference (FIG. 77D). Thus, sshLNPs manufactured by the large-scale method developed here are physically and functionally equivalent to those produced via the original dehydration-rehydration process.

### Discussion, in Particular of Examples 17 and 18

Clinical translation to realize the full potential of lipid nanoparticles requires a reliable manufacturing process capable of producing particles with the relevant properties on a multikilogram scale. Examples 17 and 18 demonstrate such a process.

The convergent manufacturing approach produces platform intermediate (i.e., “empty”) hybrid silicon nanoparticles amenable to subsequent nucleic acid loading prior to fill/finish operations that can be separated by considerable time and distance from original manufacture. This capability is conferred by the presence of silicon nanoparticles, some of which remain accessible at the surface and stabilize the lipid membrane. Their presence mitigates the well-known tendency for LNP aggregation or fusion over time (i.e., Ostwald ripening), which is a limiting factor for shelf life of current formulations (Gindy et al., 2014; Nag et al., 2022). Incorporation of SiNPs also allows for optional omission of the cholesterol component of conventional LNPs that accounts for ~40 mol % of the lipid content in marketed RNA-LNP products (Sun et al., 2023). In hybrid lipid particles of the invention, the interaction of the phospholipids with silicon provides enhanced structural integrity, reducing the risk of rupture during extrusion. Intriguingly, it also appears to produce final particles with an incompletely sealed lipid bilayer where the interior remains accessible for nucleic acid loading.

Another advantage of the silicon component is that it permits more flexibility in formulation, in the sense that the lipid composition can readily be modified without having to make significant adjustments to the manufacturing process. For example, hybrid lipid particles can be formulated without the PEGylated lipid which is an essential component of conventional LNPs, but is sometimes associated with safety and efficacy concerns. It is well known that PEGylation can induce anti-PEG antibodies, potentially provoking premature release of the RNA payload through antibody binding (Shi et al., 2022; Senti et al., 2022). Thus, non-PEGylated hybrid lipid particles may be a clinically useful option for individuals with a history of PEG hypersensitivity reactions (Chen et al., 2023; Ibrahim et al., 2022).

Moreover, the compatibility of hybrid lipid particles of the invention with point-of-care RNA loading minimizes degradation of nucleic acid drugs while providing the opportunity for late-stage customization and personalization of therapeutic formulations. In fact, this “post-hoc loading” concept has recently been highlighted as a potential strategy toward overcoming remaining challenges in the RNA medicines field (Li et al., 2023). Low delivery efficiency, short shelf life, and high market barriers to entry (i.e., cost of development and manufacture of optimized LNP formulations) are particularly recognized as current limitations that hinder the growth of the field (Verma et al., 2023), and sshLNPs can potentially answer all of these challenges.

Since 2020, priority has been given to addressing the cold chain requirements and limited shelf life encountered with COVID-19 vaccines. Notable recent innovations have resulted in improved lyophilized mRNA-LNP formulations that maintain stability and transfection efficiency at 4° C. (or even room temperature) for several weeks (Ai et al., 2023; Meulewaeter et al., 2023; Shirane et al., 2023). However, commercial scalability of these workflows has yet to be established, and they do not resolve the issue of having to incorporate the RNA component early in the production process. Extensive optimization of the LNP formulation may therefore still be necessary on a case-by-case basis. As an example, identifying the optimal formulation for patisiran—the first FDA-approved RNA therapeutic delivered by LNPs-required screening of over 300 ionizable lipids alone (Kulkarni et al., 2019). In contrast, our study with ADO2 mice required an initial screen of just seven sshLNP formulations to identify a promising lead candidate for siRNA delivery to bone (Maurizi et al., 2022). Thus, the Bio-Courier platform shows promise for accelerated clinical translation of RNA therapeutics.

Building on this point, the stabilizing effect of hybrid lipid particles of the invention is also strong enough to render chemical modification of the RNA unnecessary, as illustrated by the aforementioned in vivo studies (Baran-Rachwalska et al., 2020; Maurizi et al., 2023) that used unmodified siRNA. In contrast, all of the current FDA-approved oligonucleotide therapeutics contain chemical modifications (Bost et al., 2021), including the five siRNA-based products (Friedrich & Aigner, 2022). Use of unmodified (or minimally modified) RNAs with sshLNPs is another factor that could drive increased translation, by reducing the considerable the time and costs to develop current constructs that often require complex series of chemical modifications.

In sum, sshLNPs show significant promise as a platform technology for improved RNA delivery, but previously reported methods for their production are not easily amenable to scale-up.

### BIBLIOGRAPHY

Mikhail (1961), “Densities and Viscosities of Methanol-Water Mixtures,” (Journal of Chemical & Engineering Data, vol. 6, no. 4, p. 533-537, 1961.
Wang, C., Zhang, Y., and Dong, Y. (2021). Lipid nanoparticle-mRNA formulations for therapeutic applications. Acc. Chem. Res. 54, 4283-4293.
Curreri, A., Sankholkar, D., Mitragotri, S., and Zhao, Z. (2023). RNA therapeutics in the clinic. Bioeng. Transl. Med. 8, e10374.
Moss, K. H., Popova, P., Hadrup S. R., Astakhova, K., and Taskova, M. (2019). Lipid nanoparticles for delivery of therapeutic RNA oligonucleotides. Mol. Pharm. 16, 2265-2277.
Verma, M., Ozer, I., Xie, W., Gallagher, R., Teixeira, A., and Choy, M. (2023). The landscape for lipid-nanoparticle-based genomic medicines. Nat. Rev. Drug Discov. 22, 349-350.
Schoenmaker, L., Witzigmann, D., Kulkarni, J. A., Verbeke, R., Kersten, G., Jiskoot, W., and Crommelin, D. J. A. (2021). mRNA-lipid nanoparticle COVID-19 vaccines: Structure and stability. Int. J. Pharm. 601, 120586.
Bege, M., and Borbis, A. (2022). The medicinal chemistry of artificial nucleic acids and therapeutic oligonucleotides. Pharmaceuticals (Basel) 15, 909.
Egli, M., and Manoharan, M. (2019). Re-engineering RNA molecules into therapeutic agents. Acc. Chem. Res. 52, 1036-1047.
Nag, K., Sarker, M. E. H., Kumar, S., Khan, H., Chakraborty, S., Islam, M. J., Baray, J. C., Khan, M. K., Mahmud, A., Barman, U., et al. (2022). DoE-derived continuous and robust process for manufacturing of pharmaceutical-grade wide-range LNPs for RNA-vaccine/drug delivery. Sci. Rep. 12, 9394.
Cameau, E., Zhang, P., Ip, S., Mathiasson, L., and Stenklo, K. (2022). Process & analytical insights for GMP manufacturing of mRNA lipid nanoparticles. Cell Gene Ther. Insights 8, 621-635.
Catignol, P., and Lim, H. (2022). Overcoming bottlenecks in mRNA manufacturing (Pharma's Almanac). https://www.pharmasalmanac.com/articles/overcoming-bottlenecks-in-mrna-manufacturing.
Daniel, S., Kis, Z., Kontoravdi, C., and Shah, N. (2022). Quality by Design for enabling RNA platform production processes. Trends Biotechnol. 40, 1213-1228.
Andoh, E. A., and Yu, H. (2023). A two-stage decision-support approach for improving sustainable last-mile cold chain logistics operations of COVID-19 vaccines. Ann. Oper. Res. 328, 75-105.
Khairi, L. N. H. M., Fahrni, M. L., and Lazzarino, A. I. (2022). The race for global equitable access to COVID-19 vaccines. Vaccines (Basel) 10, 1306.
Saffie-Siebert, S., Gibson, A., and Dehsorkhi, A. (2023). Bio-Courier: A new delivery technology to increase the global accessibility of RNA therapeutics (Manufacturing Chemist). https://www.manufacturingchemist.com/news/article_page/Bio-Courier_a_new_delivery_technology_to_increase_the_global_accessibility_of_RNA_therapeutics/212116.
Baran-Rachwalska, P., Torabi-Pour, N., Sutera, F. M., Ahmed, M., Thomas, K., Nesbit, M. A., Welsh, M., Moore, C. B. T., and Saffie-Siebert, S. R. (2020). Topical siRNA delivery to the cornea and anterior eye by hybrid silicon-lipid nanoparticles. J. Control. Release 326, 192-202.
Maurizi, A., Patrizii, P., Teti, A., Sutera, F. M., Baran-Rachwalska, P., Burns, C., Nandi, U., Welsh, M., Torabi-Pour, N., et al. (2023). Novel hybrid silicon-lipid nanoparticles deliver a siRNA to cure autosomal dominant osteopetrosis in mice. Implications for gene therapy in humans. Mol. Ther. Nucleic Acids 33, 925-937.
Saffie-Siebert, R. S., Ahmed, M., and Sutera, F. (2023). Compositions comprising doped silicon particles, and related methods (WIPO Patent No. WO 2023/002222 A1).
Saffie-Siebert, R. S., and Sutera, F. (2023). Protection of biological species from degradation (WIPO Patent No. WO 2023/002224 A1).
Hou, X., Zaks, T., Langer, R., and Dong, Y. (2021). Lipid nanoparticles for mRNA delivery. Nat. Rev. Mater. 6, 1078-1094.
Kulkarni, J. A., Witzigmann, D., Chen, S., Cullis, P. R., and van der Meel, R. (2019). Lipid nanoparticle technology for clinical translation of siRNA therapeutics. Acc. Chem. Res. 9, 2435-2444.
Maurizi, A., Patrizii, P., Teti, A., Sutera, F. M., Hadainamrei, R., Dehsorkhi, A., and Saffie-Siebert, S. (2022). Development of a potential clinical product for treating CLCN7-dependent autosomal dominant osteopetrosis type 2 (ADO2). Bone Rep. 16, 101199.
Li, D., Liu, C., Li, Y., Tenchov, R., Sasso, J. M., Zhang, D., Li, D., Zou, L., Wang, X., and Zhou, Q. (2023). Messenger RNA-based therapeutics and vaccines: What's beyond COVID-19?ACS Pharmacol. Transl. Sci. 6, 943-969.
Ai, L., Li, Y., Zhou, L., Yao, W., Zhang, H., Hu, Z., Han, J., Wang, W., Wu, J., Xu, P., et al. (2023). Lyophilized mRNA-lipid nanoparticle vaccines with long-term stability and high antigenicity against SARS-CoV-2. Cell Discov. 9, 9.
Meulewaeter, S., Nuytten, G., Cheng, M. H. Y., De Smedt, S. C., Cullis, P. R., De Beer, T., Lentacker, I., and Verbeke, R. (2023). Continuous freeze-drying of messenger RNA lipid nanoparticles enables storage at higher temperatures. J. Control. Release 357, 149-160.
Shirane, D., Tanaka, H., Sakurai, Y., Taneichi, S., Nakai, Y., Tange, K., Ishii, I., and Akita, H. (2023). Development of an alcohol dilution-lyophilization method for the preparation of mRNA-LNPs with improved storage stability. Pharmaceutics 15, 1819.
Gindy, M. E., Feuston, B., Glass, A., Arrington, L., Haas, R. M., Schariter, J., and Stirdivant, S. M. (2014). Stabilization of Ostwald ripening in low molecular weight amino lipid nanoparticles for systemic delivery of siRNA therapeutics. Mol. Pharm. 11, 4143-4153.
Sun, H., Zhang, Y., Wang, G., Yang, W., and Xu, Y. (2023). mRNA-based therapeutics in cancer treatment. Pharmaceutics 15, 622.
Bost, J. P., Barriga, H., Holme, M. N., Gallud, A., Maugeri, M., Gupta, D., Lehto, T., Valadi, H., Esbjörner, E. K., et al. (2021). Delivery of oligonucleotide therapeutics: Chemical modifications, lipid nanoparticles, and extracellular vesicles. ACS Nano 15, 13993-14021.
Friedrich, M., and Aigner, A. (2022). Therapeutic siRNA: State-of-the-art and future perspectives. BioDrugs 36, 549-571.
Gottlieb, H. E., Kotlyar, V., and Nudelman, A. (1997). NMR chemical shifts of common laboratory solvents as trace impurities. J. Org. Chem. 62, 7512-7515.
Shi, D., Beasock, D., Fessler, A., Szebeni, J., Ljubimova, J. Y., Afonin, K. A., and Dobrovolskaia, M. A. (2022). To PEGylate or not to PEGylate: Immunological properties of nanomedicine's most popular component, polyethylene glycol and its alternatives. Adv. Drug. Deliv. Rev. 180, 114079.
Senti, M. E., de Jongh, C. A., Dijkxhoorn, K., Verhoef, J. J. F., Szebeni, J., Storm, G., Hack, C. E., Schiffelers, R. M., Fens, M. H., and Boross, P. (2022). Anti-PEG antibodies compromise the integrity of PEGylated lipid-based nanoparticles via complement. J. Control. Release 341, 475-486.
Chen, W.-A., Chang, D.-Y., Chen, B.-M., Lin, Y.-C., Barenholz, Y., and Roffler, S. R. (2023). Antibodies against poly(ethylene glycol) activate innate immune cells and induce hypersensitivity reactions to PEGylated nanomedicines. ACS Nano 17, 5757-5772.
Ibrahim, M., Ramadan, E., Elsadek, M. E., Emam, S. E., Shimizu, T., Ando, H., Ishima, Y., Elgarhy, O. H., Sarhan, H. A., Hussein, A. K., et al. (2022). Polyethylene glycol (PEG): The nature, immunogenicity, and role in the hypersensitivity of PEGylated products. J. Control. Release 351, 215-230.

1. A method of manufacturing an aqueous suspension of hybrid lipid particles comprising the steps of:
A. mixing one or more lipids in a solvent or solvent mixture and a suspension of particles of inorganic material in a solvent or solvent mixture, into an aqueous medium; then
B. passing the mixture resulting from Step A through the pores of an extrusion membrane,
wherein the hybrid lipid particles have a mean diameter at least two times larger than the mean diameter of the particles of inorganic material.

2. The method of claim 1, wherein the particles of inorganic material comprise or consist of particles comprising or consisting of hydrolysable silicon.

3. The method of claim 1, wherein Step B and optionally Step A are carried out at between 50° C. and 70° C.

4. The method of claim 1, wherein the particles of inorganic material are configured to impart tensile strength to one or more lipid films in the hybrid lipid particles.

5. The method of claim 1, wherein the mixture is passed through pores of an extrusion membrane at least three times and the pore size cut-off of the membrane is between 0.05 μm and 1 μm in diameter, optionally without substantially altering the size, PDI and charge of the particles.

6. The method of claim 1, wherein subsequent to Step B there is an additional Step C of purifying and/or sterilizing the suspension by tangential flow filtration, wherein Step C optionally removes activating solvent and or the solvent of step A and wherein the tangential flow filtration is optionally diafiltration using a diafiltration solution containing an amino acid such as glucine and a disaccharide such as trehalose.

7. The method of claim 1, wherein subsequent to Step B, there is an additional Step D of contacting the micellar lipid particles with an active compound.

8. The method of claim 7, wherein Step D is carried out at a temperature of below 20° C. and the active compound is an RNA molecule.

9. The method of claim 1, further comprising freeze drying the suspension to produce a lyophilized powder comprising micellar lipid particles.

10. The method of claim 1, wherein the one or more lipids comprise at least one cationic lipid and at least one polar lipid.

11. The method of claim 10, wherein the one or more lipids consist of at least DOTAP, DOPE and mPEG2000-DSPE, optionally in molecular ratios of 2 to 7:2 to 7:1 to 2.

12. The method of claim 1, wherein the particles of inorganic material are particles of hydrolysable silicon which have been activated by exposure to an alcohol such as methanol, ethanol or benzyl alcohol.

13. The method of claim 1, wherein the method does not contain a step of solvent evaporation.

14. (canceled)

15. An aqueous suspension of hybrid lipid particles having a mean diameter of between 50 and 150 nm, the hybrid lipid particles comprising a mixture of one or more cationic lipids or ionisable lipids with one or more further lipids selected from neutral lipids and polar lipids, and optionally one or more additional lipid components, the hybrid lipid particles comprising particles of inorganic material (for example, hydrolysable silicon particles) having a mean diameter of at least one half of the mean diameter of the hybrid lipid particles, wherein the weight ratio of the particles of inorganic material to lipid is between 1:2 and 1:100.

16. An aqueous suspension of hybrid lipid particles according to claim 15, and having a mean diameter of between 80 and 200 nm, wherein the particles of inorganic material are hydrolysable silicon particles which have a mean diameter of between 2 and 20 nm and are present in one or more aggregated chains of hydrolysable lipid particles which extend from the exterior of the Hybrid lipid particles to the interior of the hybrid lipid particles.

17. An aqueous suspension of hybrid lipid particles according to claim 15 further comprise one or more active compounds.

18. (canceled)

19. A method of manufacturing a liposomal lipid particle comprising an active compound, comprising carrying out a method according to claim 1, followed by contacting, in aqueous suspension the active compound with the hybrid lipid particles under conditions such that the hybrid lipid particles are transformed into liposomal lipid particles.

20. A lyophilized powder of hybrid lipid particles or liposomal lipid particles having a mean diameter of between 50 and 150 nm, the hybrid lipid particles or liposomal lipid particles comprising a mixture of one or more cationic lipids or ionisable lipids with one or more further lipids selected from neutral lipids and polar lipids, and optionally one or more additional lipid components, the hybrid lipid particles or the liposomal lipid particles comprising particles of inorganic material having a mean diameter of at least one half of the mean diameter of the liposomal lipid particles, wherein the weight ratio of the particles of inorganic material to lipid is between 1:2 and 1:100 and wherein the hybrid lipid particles or liposomal lipid particles further comprise one or more active compounds, at least a portion of which are encapsulated within the interior of the hybrid lipid particles or liposomal particle.

21. (canceled)

22. (canceled)

23. A pharmaceutical composition comprising an aqueous suspension of hybrid lipid particles according to claim 15.

24. (canceled)

25. (canceled)

26. (canceled)

27. (canceled)

28. A method of treating or preventing a disease or disorder in a subject in need thereof comprising administering to said subject a pharmaceutical composition of claim 23.

29. (canceled)

30. (canceled)

31. (canceled)

32. (canceled)

33. (canceled)
